(19) United States
(12) Patent Application Publication
(10) Pub. No.: US 2024/0269303 A1
(43) Pub. Date: Aug. 15, 2024

(54) CELL TARGETING CONSTRUCTS AND USES THEREOF

(71) Applicant: Research Development Foundation, Carson City, NV (US)

(72) Inventors: Stephen HOWELL, La Jolla, CA (US); Maria Carmen MULERO ROIG, South San Francisco, CA (US); Dennis P. GATELY, South San Francisco, CA (US)

(73) Assignee: Research Development Foundation, Carson City, NV (US)

(21) Appl. No.: 18/494,052

(22) Filed: Oct. 25, 2023

Related U.S. Application Data
(60) Provisional application No. 63/381,050, filed on Oct. 26, 2022.
(60) Provisional application No. 63/386,777, filed on Dec. 9, 2022.
(60) Provisional application No. 63/478,428, filed on Jan. 4, 2023.

(51) Int. Cl. A61K 47/68; A61K 47/64; A61P 35/00

(52) U.S. Cl. CPC A61K 47/68; A61K 47/64; A61P 35/00

(57) ABSTRACT

Cell-targeted cytotoxic compounds are provided herein. Such compounds can be used, e.g., to selectively bind and kill cancer cells such as cancer stem cells that express LGR4, LGR5, or LGR6. In some embodiments, the cytotoxic compounds include an LGR binding polypeptide, a cytotoxic agent (e.g., MMAE), and an Fc region (e.g., a mutant Fc domain). Methods for using the compounds to treat cell proliferative diseases such as cancers are also provided.

This application claims the benefit of U.S. Provisional Patent Application No. 63/381,050 filed Oct. 26, 2022, U.S. Provisional Patent Application No. 63/386,777 filed Dec. 9, 2022, and U.S. Provisional Patent Application No. 63/478,428 filed Jan. 4, 2023, the entirety of which are incorporated herein by reference.

The invention was made with government support under Grant No. CA023100 awarded by the National Institutes of Health. The government has certain rights in the invention.

This application contains a ST.26 XML Sequence Listing, which has been submitted electronically and is hereby incorporated by reference in its entirety. The Sequence Listing was created on Oct. 24, 2023, is named CLFR.P0508WO_ST26 and is 100 KB.

### BACKGROUND

### 1. Field

The present disclosure relates generally to the field of molecular biology and medicine. More particularly, it concerns cell-targeted cytotoxic constructs.

### 2. Description of Related Art

Immunotoxins and immunoconjugates have shown clinical promise for the treatment of diseases such as cancer, but significant clinical limitations remain. The successful development of targeted therapeutics, e.g., for cancer applications, depends on the identification of ligands and antigens specific for target cells, generation of molecules capable of targeting those components specifically and, finally, use of highly toxic molecules for killing of target cells. Immunoconjugates composed of antibodies and small, toxic drugs or radioisotopes have been successfully tested in vitro, in animal models and have demonstrated activity in the clinical setting. In addition to the use of small molecules for the toxin component, several highly cytotoxic protein components, such as diphtheria toxin, ricin A-chain, Pseudomonas exotoxin, and gelonin (rGel), have been used for targeted therapies. However, problems such as capillary leak syndrome, immunogenicity and inadvertent toxicity (to non-targeted cells) continue to limit implementation of successful therapy, especially for long-term or chronic applications.

Treatment of cancers that include cancer stem cells (CSC) provides a particularly challenging clinical problem. CSC are typically a small subpopulation of cells within tumors with capabilities of self-renewal, differentiation, and tumorigenicity when transplanted into an animal host, and a number of cell surface markers such as CD44, CD24, and CD133 are often used to identify and enrich CSC from tumors (Yu et al., 2012). CSCs are often resistant to conventional chemotherapy and radiation treatment, and CSC may contribute to the origin of cancer metastasis. CSC remain a significant clinical problem for the treatment of cancer and the development of approaches to kill CSC is a major goal in the field of oncology. There remains a need for highly specific and highly active toxin molecules, and cell-targeting moieties comprising such molecules, that display improved therapeutic efficacy and pharmacological properties. In particular, a need exists for improved treatment of cancers whose growth depends on CSC.

### SUMMARY

The present disclosure overcomes limitations in the prior art, in some aspects, by providing improved constructs that can be used to deliver a toxin to a cell (e.g., a cancerous cell) and display improved therapeutic properties (e.g., improved potency) and/or improved pharmacokinetic properties (e.g., improved half-life). In some aspects, constructs or polypeptides are provided that include an LGR targeting moiety or R-spondin targeting moiety (e.g., Fu1-Fu2 domain) and a cytotoxic payload (e.g., MMAE, deruxtecan, PNU159682). A variety of cytotoxic payloads can be used (e.g., FIGS. 25-27, 29), and multiple cytotoxic payloads can be included in the construct (e.g., FIGS. 30-32). The constructs may further contain a mutant Fc domain (e.g., a “DHS” mutant), and the Fc may dimerize the construct (e.g., see the schematic in FIG. 1). Since constructs provided herein may target or selectively bind an LGR (e.g., LGR4, LGR5, LGR6), they may be particularly useful for killing cancer stem cells and/or for treating cancers that comprise cancer stem cells (e.g., FIGS. 3A-E, FIG. 4). The LGR targeting moiety may also selectively bind ZNRF3 and/or RNF43. As shown in the below examples, the constructs can exhibit improved potency and half-life, and mutant forms of the LGR targeting moiety (e.g., R28A) and Fc hinge region (e.g., DHS Fc) are provided that were observed to increase the plasma half-life of the construct in vivo without altering the potency of the construct (e.g., FIGS. 28-29). One or more additional cytotoxic moieties can be covalently attached to the construct using sortase A, sortase E, and/or partial reduction of one or more disulfides in the LGR binding moiety or the Fc hinge region, to covalently bind the additional cytotoxic moiety to the construct. Anti-cancer effects of constructs provided herein were observed in vivo using multiple animal models of cancer, including in vivo xenograft animal models of colorectal cancer, gastric cancer, neuroblastoma, and ovarian cancers (e.g., see FIGS. 6A-B, 7A-B, 8A-D, 9, 12A-B, 18A-B, and FIGS. 21-24). Additional copies of the LGR targeting moiety may be used to increase binding to a cancer cell expressing an LGR such as a cancer stem cell (FIG. 17). Linkers can be included in or excluded from constructs provided herein (e.g., FIGS. 19-20). In contrast to the constructs provided in Yu et al. (2021), anticancer compounds are provided herein that are both chemically distinct and further can provide advantages in the potency, efficacy, and/or in vivo pharmacokinetic half-life; for example, as compared to R1FF-MMAE (Yu et al., 2021), the FcF2 compound provided herein surprisingly displayed a distribution half-life of ˜15-times longer than R1FF-MMAE, as measured in vivo using plasma pharmacokinetic analysis in mice (e.g., FIG. 34). Methods of treating a disease (e.g., cancer) with the constructs are also provided. Therapeutic compounds provided herein may be particularly useful for the treatment of cancers whose growth depends on CSC.

In some aspects, compounds are provided that may selectively target cancers that express R-sponin receptors LGR4, LGR5 and/or LGR6. These receptors are expressed in a variety of cells, including epithelial stem cells, normal tissues, and tumors and play important roles in embryogenesis, tissue homeostasis, and regeneration. LGR5 and LGR6 are expressed at increased levels in a variety of different types of cancers, CSC, and stem cells in the ovarian surface and fallopian tube epithelia from which ovarian cancer arises. High grade serous ovarian cancer is one of the tumors that expresses unusually high levels of LGR5 and LGR6 mRNA. R-spondins are the natural ligands for LGR5 and LGR6 to which they bind with nanomolar affinity. As shown in the below examples, in order to target cancer stem cells (e.g., in ovarian cancer and other tumors), the sortase reaction was used to site-specifically conjugate the potent cytotoxin monomethyl auristatin E (MMAE) to the two furin-like domains of RSPO1 (Fu₁-Fu₂) that mediate its binding to LGR5 and LGR6 and their co-receptors ZNRF3 and RNF43 via a protease-cleavable linker, and an immunoglobulin Fc domain was included at the N-terminal end and served to dimerize the receptor-binding domains so that each molecule carries two MMAE. The resulting molecule, FcF2-MMAE, demonstrated: 1) selective LGR5-dependent low nanomolar cytotoxicity against ovarian cancer cells in vitro; 2) selectivity that was dependent on binding to both the LGR receptors and ubiquitin ligase co-receptors; 3) favorable stability and plasma pharmacokinetic properties when administered IV with an elimination half-life of 29.7 h; 4) selective inhibition of LGR5-rich as opposed to isogenic LGR5-poor tumors in vivo; and, 5) therapeutic efficacy in two different aggressive wild type human ovarian cancer xenograft models. FcF2-MMAE can provide benefits over previously generated R1FF-MMAE (Yu et al., 2021) including: a) increasing the yield of protein from transiently transfected cultures by taking advantage of the chaperone function of Fc to improve folding; b) dimerizing the resulting molecule so that it carries two molecules of MMAE rather than one; c) increasing plasma half-life by including a mutant form of Fc with improved FcRn binding characteristics; and, d) increasing avidity of binding to the LGRs and ZNRF3/RNF43 by including 2 rather than just one copy of the Fu1-Fu2 (FuFu) domain. Achievement of these goals was evidenced by a markedly greater yield of the FcF2-His precursor, greater potency and selectivity when tested in the isogenic OVCAR8/EV and OVCAR8/LGR5 cells, a 6-fold increase in the terminal plasma half-life and improved efficacy in xenograft models. Importantly, dimerization of the two Fu1-Fu2 domains caused by the presence of the Fc domain did not compromise the efficiency of the sortase reaction which remained high. These results support the idea that Fu₁-Fu₂ domain of RSPO1 can function as a drug carrier for targeted delivery of a therapeutic compound, and FcF2-MMAE may selectively target cells in tumors that express stem cell markers. Without wishing to be bound by any theory, the results are consistent with the idea that the Fu1-Fu2 portion of the compound can simultaneously engage both (i) an LGR (e.g., LGR4 or LGR5 or LGR6) and (ii) ZNRF3 or RNF43. Exemplary amino acid sequences and nucleotides encoding polypeptides comprised in a therapeutic compound of the present disclosure are also shown, e.g., in FIGS. 13-16. In some embodiments, compounds are provided herein that include a polypeptide including (in an N-terminal to C-terminal direction): a Fc domain (e.g., a mutated Fc domain such as SEQ ID NO:13) and SEQ ID NO:75; wherein the polypeptide is covalently bonded to a cytotoxic moiety (e.g., valine-citrulline-PABA-MMAE).

An aspect of the present disclosure relates to a compound comprising one or more cytotoxic agent(s) conjugated to a polypeptide comprising one or more LGR binding domain(s), wherein (i) the polypeptide further comprises an Fc region, and/or (ii) the polypeptide comprises at least two copies of the LGR binding domain; and wherein each LGR binding domain comprises a polypeptide having at least 95% sequence identity to at least one of SEQ ID NOs: 4, 78-83, 85-89, 90-96, 98 or 102. The LGR binding domain(s) may each comprise an amino acid sequence independently selected from SEQ ID NO:4, SEQ ID NO:85, SEQ ID NO:86, or SEQ ID NO:87. In some embodiments, the LGR binding domain(s) is from human R-spondin-1 (hR-spondin-1), human R-spondin-2 (hR-spondin-2), human R-spondin-3 (hR-spondin-3), or human R-spondin-4 (hR-spondin-4). The LGR binding domain(s) may comprise an amino acid sequence independently selected from FuFu (SEQ ID NO:4) or FuFu N137A (SEQ ID NO:17). The LGR binding domain may comprise a substitution mutation at position R28 mutation or R30 with numbering according to Kabat. The substitution mutation may be arginine to alanine. In some embodiments, the substitution mutation is R28A. The LGR binding domain may comprise Fu1-Fu2 (R30A) mutant (SEQ ID NO: 91) or the Fu1-Fu2 (R30A) mutant (SEQ ID NO: 100). In some embodiments, the LGR binding domain comprises Fu1-Fu2 (R30A) mutant (SEQ ID NO: 100). The LGR binding domain may comprise Fu1-Fu2 (R22-R31 deletion) (SEQ ID NO: 92), Fu1-Fu2 (K25-R31 deletion) (SEQ ID NO: 93), Fu1-Fu2 (R28-R31 deletion) (SEQ ID NO: 94), or Fu1-Fu2 (R22-K27 deletion) (SEQ ID NO: 95). The polypeptide may comprise FcST4 (SEQ ID NO: 105). In some embodiments, the Fc region is N-terminal relative to the LGR binding domain, or wherein the polypeptide comprises in an N-to-C direction: the Fc region and the LGR binding domain. In some embodiments, the Fc region is an IgG Fc domain. The polypeptide may comprise SEQ ID NO:9, SEQ ID NO:10, SEQ ID NO:11, or SEQ ID NO:12. In some embodiments, the polypeptide comprises SEQ ID NO:78, SEQ ID NO: 80, SEQ ID NO: 82, or SEQ ID NO:88; and wherein the polypeptide does not comprise SEQ ID NO: 79, SEQ ID NO:81, SEQ ID NO:83, or SEQ ID NO:89. In some embodiments, the polypeptide comprises SEQ ID NO: 79, SEQ ID NO:81, SEQ ID NO:83, or SEQ ID NO:89; and wherein the polypeptide does not comprise SEQ ID NO:78, SEQ ID NO: 80, SEQ ID NO: 82, or SEQ ID NO:88. In some embodiments, the human IgG Fc domain capable of binding human FcRn at an acidic pH, wherein the Fc domain has substitution mutations of: (i) aspartic acid at position 309 (L/V309D); (ii) histidine at position 311 (Q311H); and (iii) a substitution mutation at position 434 of serine (N434S) or tyrosine (N434Y); with amino acid position numbering being according to the Kabat system; wherein the Fc domain binds FcRn at an acidic pH with an affinity higher than the wild-type. In some embodiments, the substitution mutation at position 434 is serine (N434S) or tyrosine (N434Y). The polypeptide may comprise SEQ ID NO:13, SEQ ID NO:14, SEQ ID NO:15, or SEQ ID NO:16. The polypeptide may comprise SEQ ID NO:13. The Fc domain may be glycosylated. The Fc domain may have the same or essentially the same binding affinity to FcγR as compared to wild-type Fc domain. The Fc domain may have the same or essentially the same binding affinity to 1, 2, or all of FcγRI, FcγRII, and FcγRIII, as compared to wild-type. In some embodiments, the Fc domain does not detectably or selectively bind to FcRn at neutral pH, and/or exhibits no or essentially no binding to FcRn at neutral pH. In some embodiments, the Fc domain exhibits: (i) enhanced binding at pH 5.8 and (ii) reduced binding or no detectable binding at pH 7.4 for FcRn, as compared to the wild-type. The Fc domain may be aglycosylated. In some embodiments, the Fc domain has a substitution mutation of glutamic acid at position 264 (V264E). The IgG may be IgG1, IgG2, IgG3, or IgG4. In some embodiments, the IgG is IgG1. In some embodiments, the Fc domain comprises substitution mutations: (i) IgG1-Fc EDHS (V264E; L309D; Q311H; N434S), (ii) IgG1-Fc EDHY (V264E; L309D; Q311H; N434Y), (iii) IgG1-Fc DHS (L309D; Q311H; N434S), (iv) IgG1-Fc DHY (L309D; Q311H; N434Y), (v) IgG2-DHS (V309D; Q311H; N434S), (vi) IgG3-DHS (L309D; Q311H; N434S), or (vii) IgG4-DHS (L309D; Q311H; N434S). The Fc domain may comprise or consist of IgG1-Fc DHS (L309D; Q311H; N434S). The compound may be dimerized via disulfide bonds formed in the Fc domain. The Fc domain may be separated from the LGR binding domain by a linker. The linker may comprise G₄S (SEQ ID NO:18) or (G₄S)₂ (SEQ ID NO:5). In some embodiments, the Fc domain is not separated from the LGR binding domain by a linker, or wherein the polypeptide does not comprise a linker. The polypeptide may comprise from the N-terminal to C-terminal ends: the Fc domain and the LGR binding domain; or wherein Fc domain is closer to the N-terminal of the polypeptide than the LGR binding domain. The compound may comprise two copies of FuFu (SEQ ID NO:4) or FuFu N137A (SEQ ID NO:17). The two copies of FuFu (SEQ ID NO:4) or FuFu N137A (SEQ ID NO:17) may be separated via a linker, preferably a G₄S linker (SEQ ID NO:18) or a (G₄S)₂ linker (SEQ ID NO:5). In some embodiments, the compound comprises SEQ ID NO: 1, SEQ ID NO:2, SEQ ID NO:3, or SEQ ID NO:19. In some embodiments, the compound comprises SEQ ID NO:1. The compound may comprise SEQ ID NO:20. In some embodiments, the polypeptide comprises a leader sequence. The leader sequence may be an endogenous leader sequence, an IgG leader sequence, or an IgK leader sequence. In some embodiments, the IgG leader sequence is IgGk leader sequence (SEQ ID NO: 8). In some embodiments, the polypeptide does not comprise a leader sequence. The cytotoxic agent may be a conjugated drug. In some embodiments, the drug is a maytansinoid, auristatin, amanitin, calicheamycin, psymberin, duocarmycin, anthracyclin, camptothecin, doxorubicin, taxol, tubulysin, eribluin, or pyrrolobenzodiazepine. The drug may be an auristatin, such as for example monomethyl auristatin E (MMAE), Monomethyl auristatin F (MMAF), or PF-06380101. In some embodiments, the auristatin is monomethyl auristatin E (MMAE). The drug may be a camptothecin analog (e.g., topotecan, irinotecan, belotecan, or deruxtecan). The drug may be an antracycline analog (e.g., PNU-159682; CAS No.: 202350-68-3). The conjugated drug may be attached to the polypeptide via a linker. The linker may be a protease-cleavable linker such as, e.g., citrulline-valine. In some embodiments, the compound comprises at least one spacer or linker (e.g., 1, 2, or 3 spacers or linkers) positioned between the cytotoxic agent and the LGR binding domain. The compound may comprise two linkers or spacers. The linker or spacer may comprise G₄S (SEQ ID NO:18) or (G₄S)₂ (SEQ ID NO:5). The cytotoxic moiety may be a cytotoxic protein such as, e.g., a serine protease. The serine protease may be a granzyme B (GrB). In some embodiments, the compound comprises SEQ ID NO:1 covalently attached to monomethyl auristatin E (MMAE). In some embodiments, the compound comprises SEQ ID NO:2 covalently attached to monomethyl auristatin E (MMAE). The compound may be dimerized via disulfide bonds in the Fc domain. In some embodiments, the cytotoxic agent has been covalently bound to the polypeptide via a sortase linker. The sortase may be a sortase A linker or a sortase E linker. In some embodiments, the compound comprises a sortase linker between the LGR binding domain and the cytotoxic agent. In some embodiments, the cytotoxic agent has been covalently bound to the polypeptide via a sortase, such as by having the sortase catalyze the covalent bonding of the cytotoxic agent to the polypeptide. The sortase may be a sortase A linker or sortase E. The sortase linker may comprise the sequence LPXT(G)ₙ, wherein n=1-10 or any range derivable therein (e.g., n=4-9), or LAHTGG (SEQ ID NO: 106). In some embodiments, the sortase linker is LPETGG (SEQ ID NO:6). The compound may further comprise a second cytotoxic agent. The first cytotoxic agent and the second cytotoxic agent may each independently be selected from the cytotoxic agents described above or herein. The first cytotoxic agent and the second cytotoxic agent may be covalently bound to the polypeptide via a sortase linker. In some embodiments, the first cytotoxic agent is covalently attached to a first sortase linker on the N-terminal side of the polypeptide, and wherein the second cytotoxic agent is covalently attached to a second sortase linker on the N-terminal side of the polypeptide. The first sortase linker comprises the sequence LPXT(G)ₙ, wherein n=1-10. The first sortase linker may be LPETGG (SEQ ID NO:6). The second sortase linker may be LAHTGG (SEQ ID NO: 106). In some embodiments, the first cytotoxic moiety has been covalently bound to the first sortase linker using sortase A, and wherein the second cytotoxic moiety has been covalently bound to the second sortase linker using sortase E. In some embodiments, the first cytotoxic moiety and the second cytotoxic moiety are each independently a conjugated drug as described above or herein, or a cytotoxic protein described above or herein. The first cytotoxic moiety and the second cytotoxic moiety may be different conjugated drugs. The first cytotoxic moiety and the second cytotoxic moiety may have the same structure. In some preferred embodiments, the first cytotoxic moiety and the second cytotoxic moiety are both monomethyl auristatin E (MMAE). In some embodiments, the first cytotoxic agent or the second cytotoxic agent are attached to the polypeptide via a disulfide bond, preferably wherein the disulfide bond is present in the Fc region or the LGR binding domain. The disulfide bond may be comprised in a maleimide group. The maleimide group may be covalently bound to a cleavable linker. The cleavable linker may comprise a valine (Val)-citrulline (Cit) bond. In some embodiments, the first cytotoxic agent is covalently bound to the polypeptide to a sortase linker comprises the sequence LPXT(G)ₙ or LAHTGG (SEQ ID NO: 106), wherein n=1-10; and wherein the second cytotoxic agent has been attached to the polypeptide via a disulfide bond. The disulfide bond may be present in the Fc region. The disulfide bond may be present in the LGR binding domain. The polypeptide may comprise SEQ ID NO:76. In some embodiments, the polypeptide comprises SEQ ID NO:77. In some embodiments, the polypeptide is covalently attached to -PABA-MMAE. The compound is comprised in a pharmaceutical composition. The pharmaceutical composition may be formulated for intravenous, intraperitoneal, subcutaneous, intratumoral, intrathecal, inhalational, intra-arterial, or intra-pleural administration.

Another aspect of the present disclosure relates to a pharmaceutical composition comprising a compound as described above of herein. The pharmaceutical composition may be formulated for intravenous, intraperitoneal, subcutaneous, intratumoral, intrathecal, inhalational, intra-arterial, or intra-pleural administration.

Yet another aspect of the present disclosure relates to a nucleic acid encoding the polypeptide described above or herein.

Another aspect of the present disclosure relates to a host cell comprising the nucleic acid described above or herein. The cell may be a bacterial cell. The cell may be a eukaryotic cell (e.g., a human cell, an insect cell, or a yeast cell). The human cell may be a HEK293 cell, Chinese Hamster Ovary (CHO) cell, or a variant thereof.

Yet another aspect of the present disclosure relates to a method of producing a therapeutic compound that binds an LGR receptor, wherein the method comprises: (a) expressing the polypeptide encoded by the nucleic acid as described above or herein in a cell, wherein the polypeptide comprises a sortase linker at a terminal end of the polypeptide; (b) obtaining the polypeptide; and (c) contacting a cytotoxic agent and the polypeptide with a first transpeptidase, thereby covalently bonding the cytotoxic compound to the polypeptide. The cell may be a bacterial cell or a eukaryotic cell (e.g., a mammalian cell or an insect cell). The mammalian cell may be a HEK293 cell, Chinese Hamster Ovary (CHO) cell, or a variant thereof. In some embodiments, the first transpeptidase is sortase A or sortase E. In some embodiments, prior to step (c) the cytotoxic moiety comprises a C-terminal sortase donor sequence and the polypeptide comprises an N-terminal sortase acceptor sequence. The C-terminal sortase donor sequence may be LPXT(G)ₙ, wherein n=1-10. The C-terminal sortase donor sequence may be LPETGG (SEQ ID NO:6). The sortase linker may further comprise -(His)ₙ-; wherein n=1-10, preferably n=4-9. The N-terminal sortase acceptor sequence may comprise 1 to 10 glycine residues. In some embodiments, the N-terminal sortase acceptor sequence is GGG. The cytotoxic agent may be a conjugated drug such as, e.g., a maytansinoid, auristatin, amanitin, calicheamycin, psymberin, duocarmycin, anthracyclin, camptothecin, doxorubicin, taxol, tubulysin, eribluin, or pyrrolobenzodiazepine. In some embodiments, the drug is an auristatin. The auristatin may be monomethyl auristatin E (MMAE), Monomethyl auristatin F (MMAF), or PF-06380101. In some embodiments, the auristatin is monomethyl auristatin E (MMAE). The drug may be a camptothecin analog (e.g., topotecan, irinotecan, belotecan, or deruxtecan). The drug may be an antracycline analog (e.g., PNU-159682; CAS No.: 202350-68-3). In some embodiments, the LGR receptor is LGR4, LGR5, or LGR6. Prior to step (c) the cytotoxic moiety may comprise an N-terminal sortase donor sequence and the polypeptide may comprise a C-terminal sortase acceptor sequence. The C-terminal sortase donor sequence may be LAHTGG (SEQ ID NO: 106). The N-terminal sortase acceptor sequence may comprise 1 to 10 glycine residues. The N-terminal sortase acceptor sequence can be, e.g., GG or GGG. The cytotoxic agent may be a conjugated drug, e.g., as described above or herein. The conjugated drug may be monomethyl auristatin E (MMAE), PNU-159682, topotecan, irinotecan, belotecan, or deruxtecan. The method may further comprise (d) covalently bonding a second cytotoxic compound to the polypeptide by either: (i) contacting a second cytotoxic agent and the polypeptide with a second transpeptidase, or (ii) covalently binding the second cytotoxic agent to the polypeptide via a partial disulfide reaction to form a disulfide bond. The partial disulfide reaction may bond the second cytotoxic agent to the Fc region or the LGR binding domain. The second cytotoxic compound may comprises a linker, and wherein the partial disulfide reaction may bind the linker to the polypeptide. The linker may comprise a thiol-reactive maleimide group. The linker may further comprise a cleavable bond. The cleavable bond may comprise a valine (Val)-citrulline (Cit) bond. The second transpeptidase may preferably be sortase A or sortase E. In some preferred embodiments, wherein the first transpeptidase is sortase A and the second transpeptidase is sortase E. The second cytotoxic agent may be a cytotoxic agent described above or herein (e.g., preferably MMAE).

Another aspect of the present disclosure relates to a method of manufacturing a polypeptide comprising: (a) expressing a nucleic acid described above or herein in a cell under conditions to produce the encoded polypeptide; and (b) purifying the polypeptide from the cell.

Yet another aspect of the present disclosure relates to a method of treating a subject with a cell proliferative disease comprising administering to the subject an effective amount of the compound described above or herein and/or the pharmaceutical composition as described above or herein. The cell proliferative disease may be an autoimmune disease. In some embodiments, the cell proliferative disease is a cancer or precancerous condition. The cancer or the precancerous condition may be characterized by the presence of cancer stem cells. The cancer stem cells may present an LGR on their surface. The LGR may be selected from LGR4, LGR5, and LGR6, preferably LGR5. The cancer may be an ovarian cancer, myeloma, lymphoma, lung cancer, breast cancer, brain cancer, prostate cancer, spleen cancer, pancreatic cancer, cervical cancer, uterine cancer, head and neck cancer, esophageal cancer, liver cancer, skin cancer, kidney cancer, leukemia, bone cancer, testicular cancer, colon cancer, basal cell carcinoma, hepatocellular carcinoma, hepatobiliary cancer, colorectal cancer, or bladder cancer. In some embodiments, the cancer is a breast cancer, ovarian cancer, endometrial cancer, colon cancer, gastric cancer, bile duct cancer, lung cancer, liver cancer, skin cancer, neuroblastoma, or leukemia. In some embodiments, the cancer is ovarian cancer or acute lymphoblastic leukemia. The cancer may be a metastatic cancer. The method may further comprise administering at least a second anticancer therapy to the subject. The second anticancer therapy may be a surgical therapy, chemotherapy, radiation therapy, gene therapy, or immunotherapy.

Another aspect of the present disclosure relates to a method for killing/treating cancer stem cells, the method comprising contacting said cancer stem cells with the compound described above or herein or with the pharmaceutical composition described above or herein.

Yet another aspect of the present disclosure relates to a method for inhibiting proliferation of cancer stem cells, the method comprising contacting said cancer stem cells with the compound described above or herein or with the pharmaceutical composition described above or herein.

Another aspect of the present disclosure relates to a method for treating cancer, the method comprising contacting said cancer stem cells with the compound described above or herein or with the pharmaceutical composition described above or herein.

Yet another aspect of the present disclosure relates to a method for reducing the spread of cancer cells and/or cancer stem cells, the method comprising contacting said cancer stem cells with the compound described above or herein or with the pharmaceutical composition described above or herein.

As used herein, “pH-selectively binding FcRn” or “binds to FcRn in a pH-selective manner” refers to a property of a polypeptide such as a Fc domain (e.g., a mutant or variant IgG Fc domain) to have the ability to bind FcRn at acidic pH (e.g., pH 5.8), and preferably the polypeptide or Fc domain has the ability to display increased binding of FcRn at acidic pH as compared to a wild-type Fc domain (e.g., a wild-type Fc IgG domain). In some embodiments, a Fc domain or polypeptide that pH-selectively binds FcRn also displays either reduced binding of FcRn at physiological pH as compared to wild-type (e.g., a wild-type IgG Fc domain) or no detectable binding of FcRn at physiological pH.

As used herein, “essentially free,” in terms of a specified component, is used herein to mean that none of the specified component has been purposefully formulated into a composition and/or is present only as a contaminant or in trace amounts. The total amount of the specified component resulting from any unintended contamination of a composition is therefore well below 0.05%, preferably below 0.01%. Most preferred is a composition in which no amount of the specified component can be detected with standard analytical methods.

As used herein “essentially the same binding affinity” indicates that two molecules exhibit statistically indistinguishable reversible binding to a ligand observed based on at least one binding assay or wherein the equilibrium constant for the reversible binding of the two molecules to the ligand exhibit a difference of less than 10%, less than 9%, less than 8%, less than 7%, less than 6%, less than 5%, less than 4%, less than 3%, less than 2%, or less than 1% for the K_{D} values. K_{D} values can be calculated based on observing Kₒₙ and K_{off} binding properties using standard methodologies known in the art such as, e.g., enzyme-linked immunosorbent assay (ELISA) or surface plasmon resonance (SPR).

As used herein, the term “affinity” refers to the equilibrium constant for the reversible binding of two agents and is expressed as K_{D}. Affinity of a binding domain to its target can be, for example, from about 100 nanomolar (nM) to about 0.1 nM, from about 100 nM to about 1 picomolar (pM), or from about 100 nM to about 1 femtomolar (fM); alternatively, it can be between 100 nM and 1 nM or between 0.1 nM and 10 nM. Moreover, it is contemplated that agents specifically bind when there is an affinity between the two agents that is in the affinity ranges discussed above.

As used herein, “LGR binding domain” refers to a polypeptide that can selectively bind an LGR receptor, preferably the LGR4, LGR5, or LGR6 receptor. The polypeptide may comprise natural and/or unnatural amino acids (e.g., D-amino acids). As used herein, “binding LGR4/LGR5/LGR6” or “LGR4/LGR5/LGR6 binding” refers to ability of a compound (e.g., a polypeptide) to bind or selectively bind at least one of LGR4, LGR5, or LGR6. For example, the polypeptide or compound may bind 1, 2, or all of LGR4, LGR5, or LGR6. When the polypeptide or compound binds two or all of LGR4, LGR5, or LGR6, it is not required that the compound bind the LGR with equal affinity. In some aspects, polypeptides are provided herein that selectively bind LGR4, LGR5, and LGR6.

As used herein the terms “encode” or “encoding,” with reference to a nucleic acid, are used to make the invention readily understandable by the skilled artisan; however, these terms may be used interchangeably with “comprise” or “comprising,” respectively.

As used herein the specification, “a” or “an” may mean one or more. As used herein in the claim(s), when used in conjunction with the word “comprising,” the words “a” or “an” may mean one or more than one.

The use of the term “or” in the claims is used to mean “and/or” unless explicitly indicated to refer to alternatives only or the alternatives are mutually exclusive, although the disclosure supports a definition that refers to only alternatives and “and/or.” As used herein “another” may mean at least a second or more.

Throughout this application, the term “about” is used to indicate that a value includes the inherent variation of error for the device used to measure the variable, the method being employed to determine the value, or the variation that exists among the study subjects.

Other objects, features and advantages of the present invention will become apparent from the following detailed description. It should be understood, however, that the detailed description and the specific examples, while indicating preferred embodiments of the invention, are given by way of illustration only, since various changes and modifications within the spirit and scope of the invention will become apparent to those skilled in the art from this detailed description.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein.

The patent or application file contains at least one drawing executed in color. Copies of this patent or patent application publication with color drawing(s) will be provided by the Office upon request and payment of the necessary fee.

FIG. 1: Schematic diagram of FcF2-His and its conversion to FcF2-MMAE using the sortase reaction.

FIGS. 2A-F: Analytic characterization of FcF2-His and FcF2-MMAE. FIGS. 2A-C, analysis of FcF2-His; FIGS. 2D-F, analysis of FcF2-MMAE. FIG. 2A & FIG. 2D, reverse phase HPLC analysis (C4 column); FIG. 2B & FIG. 2E, HPLC-based size exclusion analysis (SEC300 column). FIG. 2C & FIG. 2F, SDS-PAGE analysis under non-reducing and reducing conditions stained with Instant Blue.

FIGS. 3A-E: Cytotoxicity of FcF2-MMAE to cell lines. Potency and selective cytotoxicity of two representative batches of FcF2-MMAE in HEK293/EV vs HEK293/LGR5 (FIG. 3A & FIG. 3B), and two other representative batches in the OVCAR8/EV vs OVCAR8/LGR5 models (FIG. 3C & FIG. 3D). FIG. 3E, Potency of FcF2-MMAE against 8 human ovarian carcinoma cell lines expressing endogenous levels of LGRs. Each curve represents inhibition of growth during a 120 h exposure to increasing concentrations of FcF2-MMAE. Viability was determined using the CCK8 reagent.

FIG. 4: Relative contribution of the Fu₁ and Fu2 domains to the selective cytotoxicity of FcF2-MMAE. Effect of disabling the binding of either Fu₁ (FcF2-Q71R-MMAE, Fu2 (FcF2-F106R-F110R-MMAE) or both (FcF-Q71R-F106R-F110R-MMAE) on selectivity of growth inhibition when OVCAR8/EV and OVCAR8/LGR5 cells were exposed for 120 h. Values are mean±SEM of 3 independent experiments each performed with triplicate cultures.

FIGS. 5A-B: Plasma pharmacokinetics of FcF2-MMAE. FIG. 5A, Plasma concentration of FcF2-MMAE as a function of time following IV injection of 0.1 nmol/g (9 ug/g) in BALB/c mice determined by ELISA. FIG. 5B, Concentration of FcF2-MMAE in the plasma fraction of mouse whole blood as a function of time during incubation determined by ELISA.

FIGS. 6A-B: Efficacy and toxicity of FcF2-MMAE against human ovarian cancer OVCAR8/EV (LGR5-low) and OVCAR8/LGR5 (LGR5-rich) xenografts. FIG. 6A, Tumor volume as a function of time. FIG. 6B, Mouse weight during and following the course of treatment. Dose schedule: 0.5 nmol/g (42.65 mg/kg) q4dx4 IP. N=8/group. Vertical arrows indicate dose days. Vertical bars, ±SEM.

FIGS. 7A-B: Efficacy and toxicity of FcF2-MMAE as a function of dose administered IP every 7 days for 6 doses in the OVCAR8/LGR5 xenograft model. FIG. 7A, Tumor growth as a function of dose. FIG. 7B, Mouse weight during and following the course of treatment as a function of dose. Vertical arrows indicate days on which doses were delivered. Vertical bars, ±SEM.

FIGS. 8A-D: Efficacy of a single dose of 1.0 nmol/g FcF2-MMAE in the CAOV3 and KF-28 xenografts models. Average tumor volumes (FIG. 8A) and animal weights (FIG. 8B) for KF-28 xenografts. B. Average tumor volumes (FIG. 8C) and animal weights (FIG. 8D) for CAOV3. Vertical arrow indicates the day on which the dose was injected. Vertical bars, ±SEM.

FIG. 9: Efficacy of FcF2-MMAE against wild type OVCAR8 and CAOV3 xenografts at a dose of 0.5 nmol/g for 4 doses. A & C, Tumor growth as a function of time. B & D, Mouse weight during and following the course of treatment. Vertical arrows indicate days on which a dose was injected. Vertical bars, ±SEM.

FIGS. 10A-B: Documentation of differential glycosylated forms of FcF2-His. FIG. 10A, FcF2-His samples before and after treatment with PNGase run under reducing conditions and stained with Instant Blue. FIG. 10B, Characterization of FcF2-MMAE by Western blot analysis of a non-reducing gel probed with anti-RSPO1 (left) and anti-MMAE (right).

FIGS. 11A-C: Flow cytometric documentation of differential LGR5 expression in isogenic pairs of HEK293 and OVCAR8 cells and FcF2-MMAE-mediated depletion of LGR5 expressing cells. FIG. 11A, HEK293/EV vs HEK293/LGR5 cells; FIG. 11B, OVCAR8/EV vs OVCAR8/LGR5 cells (EV cells: green, secondary antibody only; red primary and secondary antibodies. LGR5 cells: purple, secondary antibody only; blue. primary and secondary antibodies). FIG. 11C, Flow cytometric analysis of a mixture of OVCAR8/EV and OVCAR8/LGR5 cells before and after exposure to 10 nm FcF2-MMAE for 120 h (gray and black curves, untreated and FcF2-MMAE-treated unstained cells; green and blue curves, untreated and treated OVCAR8/EV and OVCAR8/LGR5 cells stained with anti-LGR5).

FIGS. 12A-B: Efficacy and toxicity of FcF2-MMAE against human ovarian cancer OVCAR8/EV (LGR5-low) and OVCAR8/LGR5 (LGR5-rich) xenografts at a dose schedule: 1.5 nmol/g (128 mg/kg) q7dx4 IP. FIG. 12A, Tumor volume as a function of time. FIG. 12B, Mouse weight during and following the course of treatment. N=8/group. Vertical arrows indicate dose days. Vertical bars, ±SEM.

FIG. 13: Amino acid sequence of FcF2 containing a 8×His tag. The amino acid sequence contains an IgGkappa leader sequence followed by the DHS variant of IgG1 Fc (Lee et al., 2019) linked to the Fu1-Fu2 domains. Blue highlighted amino acids in the Fc portion of the construct are used to indicate the DHS mutations in the Fc portion. The purple arrow indicates the protease cleavage site at the end of the leader sequence. The blue arrows indicate the cysteines in the Fc region that can form disulfides with corresponding cysteines in another separate Fc region in order to form a dimerized molecule.

FIG. 14: Nucleotide sequence for producing the FcF2 construct in FIG. 13, with the difference that DHS mutations are not included in the Fc portion of the construct. The nucleotide sequence shown includes codon optimization. Purple highlighted codons within the Fc portion of the construct indicate regions that would be subsequently to be changed using GeneArt to include the DHS mutant version of the Fc region. The color coding of different regions otherwise corresponds to regions described in FIG. 13.

FIG. 15: Sequence with DHS mutations prior to codon optimization. The blue heighted codons in the Fc region now correspond to the DHS mutations in DHS Fc. The color coding of different regions otherwise corresponds to regions described in FIG. 13.

FIG. 16: FcF2-8×His nucleotide (nt) sequence after codon optimization for expression in human cells. This nucleotide sequence corresponds to the amino acid sequence shown in FIG. 13.

FIG. 17: Construction of FcST4. The amino acid sequence of FcST4 is shown (top). A vector construct for the FcST4 molecule was designed to contain an IgG leader sequence on the N terminal end, followed by a mutated Fc domain attached to two modified receptor binding domains (ST) in series with a linker sequence in between the two ST domains. A second spacer sequence was inserted just upstream of the LPETGG sortase recognition (donor) motif and an 8×His tag was positioned at the C terminal end.

FIGS. 18A-B: Analysis of the potency of FcST2-MMAE against 8 human ovarian cancer cell lines. FIG. 18A, Survival as a function of FcST2-MMAE concentration for 8 human ovarian cancer cell lines. FIG. 18B, IC₅₀ values for the 8 cell lines tested.

FIG. 19: Linkerless FcF2. The linkerless FcF2 construct is shown (bottom). In contrast to the FcF2 that includes linkers (top), the linkerless FcF2 does not include the G4S linkers in the polypeptide.

FIG. 20: Cytotoxicity of FcF2-MMAE and FcF2Δlinker-MMAE.

FIG. 21: FcF2-MMAE activity in vivo in a xenograft mouse model of human colorectal cancer. Mice bearing human colorectal xenograft (LoVo) were administered a dose that did not cause any observed clinical toxicity. FcF2-MMAE resulted in a decrease in the average weight of the tumors, without altering body weight of the mice.

FIG. 22: Efficacy of FcF2-MMAE in colon cancer LoVo xenograft model. Dose schedule: FcF2-MMAE injected IP q7dx4. Each data point is the mean of the size of all tumors in the group. Vertical bars are SEM.

FIG. 23: Efficacy of FcF2-MMAE in gastric carcinoma AGS xenograft model. Dose schedule: FcF2-MMAE injected IP q7dx4. Each data point is the mean of the size of all tumors in the group. Vertical bars are SEM.

FIG. 24: Efficacy of FcF2-MMAE in neuroblastoma SKNAS xenograft model. Dose schedule; FcF2-MMAE injected IP q7dx4. Each data point is the mean of the size of all tumors in the group. Vertical bars are SEM.

FIGS. 25A-C: Analysis of the structural integrity and selective cytotoxicity of FcF2-PNU159682. FIG. 25A, SDS-PAGE analysis of two different batches of FcF2-PNA159682. FIG. 25B, Growth inhibition of OVCAR8/EV versus OVCAR8/LGR5 cells as a function of FcF2-PNU159685 concentration (pM). Data is mean±SEM of triplicate cultures. FIG. 25C, Schematic of the structure of PNU159683.

FIGS. 26A-B: Construction of GGGC-GGFG-deruxtecan linker. FIG. 26A, Schematic of the conjugation of GGGC with maleimide-GGFG-deruxtecan. FIG. 26B, Reverse-phase HPLC analysis of GGGC-MA-GGFG-deruxtecan linker, C4 column.

FIGS. 27A-B: Production of FcF2-deruxtecan and documentation of its cytotoxicity and cytotoxic selectivity. FIG. 27A, reverse-phase HPLC (C4 column) analysis of FcF2-deruxtecan documenting purity. FIG. 27B, growth inhibition of OVCAR8/EV versus OVCAR8/LGR5 cells as a function of FcF2-deruxtecan concentration. Data is mean±SEM of triplicate cultures.

FIG. 28: Documentation that FcF2-MMAE containing mutations R28A and R30A retain potency and cytotoxic selectivity. Growth inhibition of OVCAR8/EV versus OVCAR8/LGR5 cells as a function of FcF2-MMAE (WT), FcF2-R28A-MMAE and FcF2-R30A-MMAE concentration. Data is mean±SEM of triplicate cultures.

FIG. 29: Pharmacokinetics of FcF2-MMAE (WT), FcF2-R28A-MMAE and FcF2-R30A-MMAE. A, Plasma concentration of each form as a function of time after IV injection in BALB/c mice. B, Table of Area Under the Curve, 0-120 h, for each form.

FIG. 30: Schematic for loading of MMAE on both ends of the FcF2 molecule using both sortase A and sortase E.

FIG. 31: Documentation of the loading of GGGC-MA-Dye650 onto FcF2-LAHTGG-His by sortase E. Figure shows SDS-PAGE analysis of the sortase E reaction product (lanes 7 and 8) after elution from SP-sepharose resin detected using the fluorescence of Dye650.

FIG. 32: Western blot analysis documenting incorporation of MMAE using partial reduction of disulfide bonds by TCEP. Upper panel, blot probe with anti-RSPO1 antibody; lower panel, blot probed with anti-MMAE.

FIG. 33: Nucleotide and amino acid sequences of the FcF2 (R28A) construct. Kozak Sequence is shown in light blue. IgGk Leader Sequence is shown in green highlighting. Fc sequence is shown in grey highlighting, with the DHS mutations shown with underlining and yellow highlighting. The G4SG4S linker is highlighted in red. The LPETG sequence is highlighted in purple. The 8×His-tag is highlighted in light blue. The Stop X 2 sequence is shown in dark blue highlighting. The Fu1-Fu2 (FuFu) sequence is shown in yellow highlighting. The R28A substitution mutation within FuFu is further shown in underlined white font with black highlighting.

FIG. 34: In vivo plasma distribution half-life of R1FF-MMAE (“RSPO1-MMAE”) and FcF2-MMAE in female BALB/C mice. The FcF2-MMAE construct displayed a half-life that was ˜15-times longer than R1FF-MMAE. Distribution half-life (T_{1/2}) and elimination half-life (T_{1/2}) values are shown.

### DESCRIPTION OF ILLUSTRATIVE EMBODIMENTS

The present disclosure, in some aspects, overcomes limitations in the prior art by providing constructs that selectively target and kill cancer cells, including cancer stem cells. The construct may include a polypeptide with a region that can selectively bind LGR4, LGR5, and/or LGR6 receptors expressed by the cancerous cells or CSC. The construct may also include a cytotoxic moiety (e.g., MMAE) attached to the polypeptide via a cleavable linker, such that the construct may be endocytosed into a cancerous cell and then released into the cell. Delivery of the cytotoxic moiety to a cancerous cell such as a cancer stem cell can thus result in selective killing of the cancerous cell. The present disclosure is based, in part, on the discovery that including a Fc region in the polypeptide (e.g., a mutant Fc region such as the DHS Fc mutant provided herein) can result in improvements to the pharmacological profile (e.g., improvements in the half-life of the construct in vivo) and/or improve therapeutic efficacy. Inclusion of the Fc region can also result in a dimerized construct, effectively doubling both the number of LGR binding domains and the number of cytotoxic moieties within the construct. In some aspects, the present disclosure is based on the observation that including two or more LGR binding domains within the polypeptide can result in improvements in the therapeutic properties of the construct for treating a cancer, such as a cancer that includes cancer stem cells. Methods for treating cancers and selectively targeting cancer stem cells are also provided herein.

In various aspects, therapeutic constructs are provided. The therapeutic construct may comprise (i) one or more cell targeting moiety such as an LGR5/LGR6 binding domain (e.g., Fu1-Fu2 sequence, SEQ ID NO:4; Fu1-Fu2 (N137A), SEQ ID NO:17) and (ii) a cytotoxic moiety (e.g., MMAE or deruxtecan). The therapeutic construct may for example comprise 1, 2, 3, or 4 copies of the cell-targeting moiety (e.g., Fu1-Fu2 sequence, SEQ ID NO:4; Fu1-Fu2 (N137A), SEQ ID NO:17) that may be separated by a linker (e.g., a G4S linker). In some embodiments, the therapeutic construct comprises a Fc domain, such as a wild type or mutant Fc domain (e.g., a DHS mutant Fc; SEQ ID NO:13). The Fc domain may cause the dimerization of two copies of therapeutic construct, effectively doubling the number of cell-targeting moieties and cytotoxic moieties in the dimerized construct. As shown in the below examples, inclusion of the DHS Fc domain resulted in an increase in the half-life of the construct in vivo. In some embodiments, the therapeutic construct may comprise a radioisotope, imaging agent, or a radiotherapy. The therapeutic construct may be comprised in a pharmaceutical composition such as, e.g., a formulation for injection (e.g., intravenous or intratumoral injection), inhalation, or inclusion in liposomes or nanoparticles. Also provided herein are methods of using the therapeutic constructs. In some embodiments, the therapeutic constructs may be used to treat a cancer that expresses LGR4, LGR5, or LGR6.

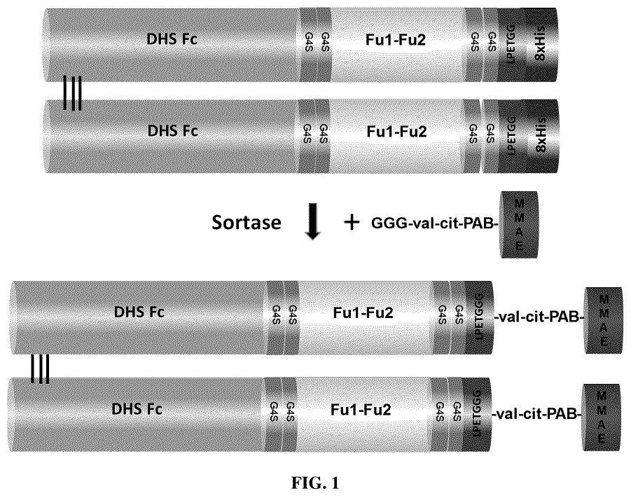

In some aspects, a therapeutic compound provided herein may include a Fu₁-Fu₂ receptor-binding domain of an R-spondin to target cancer cells that express LGR4, LGR5 and/or LGR6, such as ovarian cancer cells. As shown in the below examples, the Fu₁-Fu₂ receptor-binding domain of RSPO1 can be conjugated to or covalently bonded to monomethyl auristatin E (MMAE) to ovarian cancer cells rich in stem cell receptor LGR5. A modified IgG1 Fc domain containing half-life extending modifications (Lee et al., 2019) was linked to the N-terminal end of the Fu₁-Fu₂ domain which bore a sortase recognition sequence on its C-terminal end. During synthesis of this protein in HEK293E cells, the 2 chains were linked by intermolecular disulfide bonds between the Fc domains resulting in a dimeric molecule “FcF2-His” containing two LPETGG (SEQ ID NO:6) sequences at the C-terminal ends to which MMAE can be covalently attached or conjugated using the sortase reaction to produce “FcF2-MMAE” (e.g., FIG. 1). This molecule displayed both low nM cytotoxicity in a panel of human ovarian cancer cell lines and a favorable stability and pharmacokinetic profile. Moreover, FcF2-MMAE resulted in selective killing of LGR5-rich tumor cells in vitro and differential inhibition of the growth of isogenic LGR5-poor and LGR5-rich tumors in vivo. It exhibited activity in two different human ovarian cancer xenograft models on a clinically relevant dose schedule, and at doses that produced only transient adverse side effects. These results in ovarian cancer models illustrate that cancer stem cells expressing LGR5/LGR6 can be selectively targeted and killed using these compositions and approaches. FcF2-MMAE and other therapeutic compounds provided herein can be used to reduce the growth of and/or treat a variety of different types of cancers.

### I. LGR Binding Polypeptides

In some aspects, therapeutic compounds provided herein may comprise an LGR binding domain polypeptide that can selectively bind LGR4, LGR5 and/or LGR6. LGR5 has been observed to be expressed in cancers including basal cell carcinomas, hepatocellular carcinomas, colorectal tumors, and ovarian tumors (McClanahan et al., 2006). LGR6 is expressed in cancers including adenocarcinoma (Cortesi et al., 2019). By selectively binding to LGR4, LGR5 and/or LGR6, therapeutic constructs provided herein can target select cell types, such as cancer cells or stem cells. In some embodiments, the LGR binding domain can selectively bind LGR5 and LGR6. The LGR binding domain may be a polypeptide, which may include natural and/or non-natural amino acids.

In some embodiments, the LGR binding domain comprises or consists of a polypeptide sequence. The polypeptide may comprise a FurinL sequence. FurinL sequences are each of the two cysteine-rich, furin-like domains in an R-spondin polypeptide (De Lau et al., 2012). In some embodiments, the polypeptide contains a first FurinL sequence (also referred to as a “Fu1” or “FurinL repeat 1”) and a second FurinL sequence (also referred to as a “Fu2” or “FurinL repeat 2”). The LGR binding domain may comprise a FurinL sequence from a R-spondin protein, such as human R-spondin-1 (hR-spondin-1), human R-spondin-2 (hR-spondin-2), human R-spondin-3 (hR-spondin-3), or human R-spondin-4 (hR-spondin-4), which are described, e.g., in De Lau et al. (2012) and Jin-Gen et al. (2015). The first FurinL sequence (Fu1) and the second FurinL sequence (Fu2) may comprise or consist of the FurinL sequences from human R-spondin-1 (hR-spondin-1), human R-spondin-2 (hR-spondin-2), human R-spondin-3 (hR-spondin-3), or human R-spondin-4 (hR-spondin-4). In some embodiments, the LGR binding domain comprises or consists of a furin region (e.g., Fu1-Fu2 region) of a R-spondin protein such as human R-spondin-1 (hR-spondin-1), human R-spondin-2 (hR-spondin-2), human R-spondin-3 (hR-spondin-3), or human R-spondin-4 (hR-spondin-4). In some embodiments, the LGR binding domain is the Fu1-Fu2 sequence (SEQ ID NO:4). The LGR binding domain may be included multiple times within a therapeutic compound or polypeptide provided herein (e.g., repeated 1, 2, 3, or 4 times within a therapeutic polypeptide provided herein). In some embodiments, the LGR binding domain comprises one or more substitution mutations as compared to a LGR binding domain in a human R-spondin protein, e.g., Fu1-Fu2 (N137A) (SEQ ID NO:17). The LGR binding domain may comprise a polypeptides having at least 85, 90, 91, 92, 93, 94, 95, 96, 97, 98, 99% sequence identity to the any of the R-spondin or FurinL sequences provided above or herein.

As used herein, “Fu1-Fu2” and “FuFu” are used interchangeably herein to refer to a polypeptide that contains both a Fu1 FurinL sequence and a Fu2 FurinL sequence from an R-spondin. In some embodiments, the Fu1 region and Fu2 region may be derived from the same R-spondin polypeptide (e.g., from human RSPO1). Nonetheless, in some embodiments a polypeptide may include a Fu1 region from a first R-spondin protein and a Fu2 region from a second R-spondin protein. For example, FurinL sequences from different human R-spondin proteins, such as for example a Fu1 region from RSPO1 and a Fu2 region from RSPO2, can be included in a single polypeptide to selectively bind an LGR receptor, such as LGR4, LGR5, and/or LGR6.

In some embodiments, the LGR binding domain comprises one or more copies of the following “Fu1-Fu2” sequence (SEQ ID NO:4): SRGIKGKRQRRISAEGSQACAKGCELCSEVNGCLKCSPKLFILLERNDIRQVGVCLPSCPP GYFDARNPDMNKCIKCKIEHCEACFSHNFCTKCKEGLYLHKGRCYPACPEGSSAAAGT MECSSP; or a polypeptide having at least 90, 91, 92, 93, 94, 95, 96, 97, 98, 99, 99.5, or 99.9 sequence identity.

Other LGR binding domain may include Fu1 and/or Fu2 domains from human RSPO2, RSPO3 or RSPO4; or a polypeptide having at least 90, 91, 92, 93, 94, 95, 96, 97, 98, 99, 99.5, or 99.9 sequence identity to one of these LGR5/LGR6 binding domains (e.g., to SEQ ID NO:4 or one or SEQ ID Nos:78-83). Fu1 and Fu2 domains that can be used include:

RSPO1
RSPO1-Fu1:
(SEQ ID NO: 88)
AEGSQACAKGCELCSEVNGCLKCSPKLFILLERNDIRQVGVCLPSCPPG
YFD
RSPO1-Fu2:
(SEQ ID NO: 89)
MNKCIKCKIEHCEACFSHNFCTKCKEGLYLHKGRCYPACPEGSSA
RSPO2
RSPO2-Fu1:
(SEQ ID NO: 78)
SYVSNPICKGCLSCSKDNGCSRCQQKLFFFLRREGMRQYGECLHSCPSG
YYG
RSPO2-Fu2:
(SEQ ID NO: 79)
MNRCARCRIENCDSCFSKDFCTKCKVGFYLHRGRCFDECPDGFAP
RSPO2-Fu1-Fu2:
(SEQ ID NO: 85)
SYVSNPICKGCLSCSKDNGCSRCQQKLFFFLRREGMRQYGECLHSCPSG
YYGHRAPDMNRCARCRIENCDSCFSKDFCTKCKVGFYLHRGRCFDECPD
GFAP
RSPO3
RSPO3-Fu1:
(SEQ ID NO: 80)
PNVSQGCQGGCATCSDYNGCLSCKPRLFFALERIGMKQIGVCLSSCPSG
YYG
RSPO3-Fu2:
(SEQ ID NO: 81)
INKCTKCKADCDTCFNKNFCTKCKSGFYLHLGKCLDNCPEGLEA
RSPO3-Fu1-Fu2:
(SEQ ID NO: 86)
PNVSQGCQGGCATCSDYNGCLSCKPRLFFALERIGMKQIGVCLSSCPSG
YYGTRYPDINKCTKCKADCDTCFNKNFCTKCKSGFYLHLGKCLDNCPEG
LEA
RSPO4
RSPO4-Fu1:
(SEQ ID NO: 82)
GTGLGGNCTGCIICSEENGCSTCQQRLFLFIRREGIRQYGKCLHDCPPG
YFGIR
RSPO4-Fu2:
(SEQ ID NO: 83)
VNRCKKCGATCESCFSQDFCIRCKRQFYLYKGKCLPTCPPGTLA
RSPO4-Fu1-Fu2:
(SEQ ID NO: 87)
GTGLGGNCTGCIICSEENGCSTCQQRLFLFIRREGIRQYGKCLHDCPPG
YFGIRGQEVNRCKKCGATCESCFSQDFCIRCKRQFYLYKGKCLPTCPPG
TLA

In some embodiments, either a Fu1 or a Fu2 region may be included in a construct, instead of a polypeptide containing both a Fu1 and Fu2 region, in order to selectively bind the LGR4, LGR5, and/or LGR6 receptor. The single Fu1 or Fu2 region can be used to generate a therapeutic compound (e.g., Fc-Fu1-MMAE or Fc-Fu2-MMAE) that has cytotoxicity and selectivity for LGR4, LGR5, and/or LGR6. The single Fu1 or Fu2 region may be included in a polypeptide as described herein in combination with another cell targeting polypeptide, such as for example an scFv region. For example, a Fu1 region and an scFv (e.g., that selectively targets a liver antigen) can be included in a single polypeptide that is covalently attached to a drug (e.g., a cytotoxic moiety such as MMAE), and may selectively direct the drug to the target tissue of the scFv (e.g., the liver) while reducing or avoiding toxicity to the gut that would have been mediated by a Fu2 domain.

The LGR binding domain may contain both a Fu1 region and a Fu2 region as described herein. In some embodiments, including both the Fu1 and Fu2 region may result in constructs with increased cytotoxicity and/or selectivity, as compared to including only a single Fu1 or Fu2 region.

The Fu1-Fu2 sequence above may comprise a substitution mutation to remove the N-glycosylation site at position (N137). For example, in some embodiments, the N-glycosylation site in Fu1-Fu2 is removed by the asparagine to alanine substitution mutation at position 137 (i.e., N137A). The FuFu (N137A) sequence comprises the sequence (SEQ ID NO:17): SRGIKGKRQRRISAEGSQACAKGCELCSEVNGCLKCSPKLFILLERNDIRQVGVCLPSCPP GYFDARNPDMNKCIKCKIEHCEACFSHNFCTKCKEGLYLHKGRCYPACPEGSSAANGT MECSSP; or a polypeptide having at least 90, 91, 92, 93, 94, 95, 96, 97, 98, 99, 99.5, or 99.9 sequence identity to FuFu (N137A) (SEQ ID NO:17).

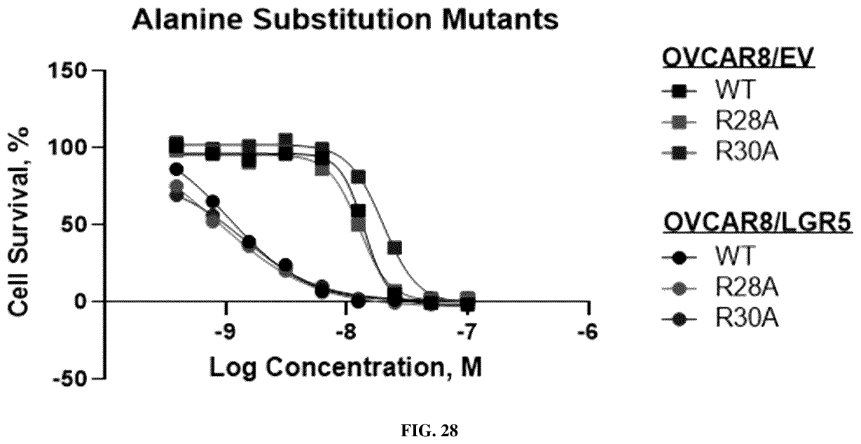

The Fu1-Fu2 sequence may comprise 1, 2, 3, 4, 5, or more substitution mutations that may provide an additional beneficial effect such as altering the pharmacokinetics or increasing the half-life of the construct after administration to a mammalian subject. For example, the Fu1-Fu2 sequence may comprise the (R28A) substitution mutation: SRGIKGKAQRRISAEGSQACAKGCELCSEVNGCLKCSPKLFILLERNDIRQVGVCLPSCPP GYFDARNPDMNKCIKCKIEHCEACFSHNFCTKCKEGLYLHKGRCYPACPEGSSAANGT MECSSP (SEQ ID NO: 90). The Fu1-Fu2 sequence may comprise the (R30A) substitution mutation: SRGIKGKRQARISAEGSQACAKGCELCSEVNGCLKCSPKLFILLERNDIRQVGVCLPSCPP GYFDARNPDMNKCIKCKIEHCEACFSHNFCTKCKEGLYLHKGRCYPACPEGSSAANGT MECSSP (SEQ ID NO: 91). As shown in Example 7 and FIG. 28, neither the R28A nor the R30A mutation altered potency of selectivity for LGR5-rich cells; however, R28A, but not the R30A, mutation prolonged the initial half-life of the molecule in vivo and increased the AUC₀₋₁₂₀ by a factor of 3.4-fold. In some preferred embodiments, the Fu1-Fu2 sequence may comprise the R28A mutation, which may result in an increase of the half-life (e.g., plasma half-life) after administration to a mammalian subject in vivo.

Deletion mutants of the FuFu sequence can also be used. The following deletion mutants may increase the half-life or plasma half-life of the construct. FuFu deletion mutants that can be used include: Fu1-Fu2 (R22-R31 deletion) SISAEGSQACAKGCELCSEVNGCLKCSPKLFILLERNDIRQVGVCLPSCPPGYFDARNPD MNKCIKCKIEHCEACFSHNFCTKCKEGLYLHKGRCYPACPEGSSAANGTMECSSP (SEQ ID NO: 92), Fu1-Fu2 (K25-R31 deletion) SRGIISAEGSQACAKGCELCSEVNGCLKCSPKLFILLERNDIRQVGVCLPSCPPGYFDARN PDMNKCIKCKIEHCEACFSHNFCTKCKEGLYLHKGRCYPACPEGSSAANGTMECSSP (SEQ ID NO: 93), Fu1-Fu2 (R28-R31 deletion) SRGIKGKISAEGSQACAKGCELCSEVNGCLKCSPKLFILLERNDIRQVGVCLPSCPPGYFD ARNPDMNKCIKCKIEHCEACFSHNFCTKCKEGLYLHKGRCYPACPEGSSAANGTMECSS P (SEQ ID NO: 94), Fu1-Fu2 (R22-K27 deletion) SRQRRISAEGSQACAKGCELCSEVNGCLKCSPKLFILLERNDIRQVGVCLPSCPPGYFDA RNPDMNKCIKCKIEHCEACFSHNFCTKCKEGLYLHKGRCYPACPEGSSAANGTMECSSP (SEQ ID NO: 95), and Fu1-Fu2 (S21-Q38 deletion) ACAKGCELCSEVNGCLKCSPKLFILLERNDIRQVGVCLPSCPPGYFDARNPDMNKCIKC KIEHCEACFSHNFCTKCKEGLYLHKGRCYPACPEGSSAANGTMECSSP (SEQ ID NO: 96).

In some embodiments, the FuFu sequence is attached to a linker (e.g., (G₄S)ₙ, wherein n=1-3), a cleavable linker (e.g., containing valine-citrulline, such as valine-citrulline-PAB), and a cytotoxic moiety (e.g., MMAE). For example, the polypeptide may comprise a Fc domain (e.g., a mutant Fc domain such as SEQ ID NO:13-16) and the FuFu region. The FuFu region may be attached to a linker and MMAE (wherein the MMAE has been covalently attached using a sortase enzyme), e.g.:

SRGIKGKRQRRISAEGSQACAKGCELCSEVNGCLKCSPKLFILLERNDI
RQVGVCLPSCPPGYFDARNPDMNKCIKCKIEHCEACFSHNFCTKCKEGL
YLHKGRCYPACPEGSSAANGTMECSSPGGGGSGGGGSLPETGGG-
valine-citrulline-PABA-MMAE (SEQ ID NO: 76-
citrulline-valine-PABA-MMAE).

In some embodiments, the cell targeting moiety comprises a polypeptide that contains at least 1, 2, 3, or 4 copies of a Fu1-Fu2 sequence (e.g., two or more copies of SEQ ID NO:4 or SEQ ID NO:17). For example, the cell targeting moiety may comprise two copies of the Fu1-Fu2 sequence (SEQ ID NOs:4). The Fu1-Fu2 sequences may be separated by a linker such as, e.g., G₄S (GGGGS, SEQ ID NO:18), (G₄S)₂ (SEQ ID NO:5), (Gly)₆, or (EAAAK)₃ (SEQ ID NO:84).

If the construct comprises a Fc region as described herein (e.g., a mutant Fc region such as DHS Fc, SEQ ID NO:13), then the construct may dimerize based on association of the Fc region in two different molecules. In this way, the total number of LGR5/LGR6 binding domain and cytotoxic agents, etc. can effectively double in a single dimerized construct. The total number of LGR5/LGR6 binding domains (e.g., Fu1-Fu2, FuFu (N137A)) in the dimerized construct may be 2, 4, 6, or 8.

The LGR family of G-protein-coupled 7-transmembrane spanning receptors contains 8 members all of which have large extracellular domains consisting of up to 18 copies of a leucine-rich repeat motif. The 8 receptors fall into 3 groups. The first consists of LGR1 which is the FSH receptor, LGR2 the LH receptor, and LGR3 the TSH receptor. The second consists of LGR4, LGR5, and LGR6 which are receptors for the R-spondins (RSPOs) and the third group contains LGR7 and LGR8 which are receptors for relaxin and the insulin-like 3 protein, respectively. LGR5 and LGR6 are the well-defined markers for stem cells in the gut (LGR5) and skin and Fallopian tube epithelium (LGR6), respectively, and in many other tissues and tumor types. LGR5 was shown to be positively regulated by the Wnt signaling pathway that controls the proliferation of the stem cells that form the epithelium of the colon, small intestine and stomach. During embryonic development LGR5 is expressed in multiple tissues, but in the adult its expression is very restricted to rare cells in the gut, breast, ovary, testis, hair follicles, brain and eye. Using a genetic marking technique, cells that express LGR5 were found to function as stem cells capable of giving rise to all the other types of cells found in the epithelium of the colon and stomach. In contrast to LGR5, LGR6 is not regulated by Wnt signaling. In LGR6-LacZ^{LacZ} knock-in mice expression was found to be limited to rare cells in the brain, breast, lung and hair follicles. Lineage mapping has shown that LGR6-positive cells residing in the bulb of the hair follicle are located in a different position than the LGR5-positive cells, and that they give rise to the epidermis and sebaceous glands. Subsequent studies demonstrated that LGR6-expressing cells are the stem cells that generate the new skin needed during the wound healing process. There is also evidence that LGR6 is uniquely expressed by tumor stem cells. LGR6 was found to mark the subpopulation of cells isolated from human lung adenocarcinomas that are capable of forming new tumors in injected into mice.

LGR5 and LGR6 are expressed in many types of tumors including cancers of the breast, colon and endometrium. There are several lines of evidence suggesting that LGR6 rather than LGR5 uniquely identifies stem cells in the Fallopian tube epithelium (FTE) and in ovarian cancers. Thus, LGR6 appears to stem cells in tumors arising from the FTE. Therefore, embodiments of the present disclosure concern the use of LGR6 as a target of tumor stem cells as it is expressed on the cell surface where it is potentially accessible to antibodies and other kinds of tumor-targeting toxins.

R-spondins (RSPOs) are ligands for LGR5 and LGR6. RSPO are a group of 4 cysteine-rich secreted paralogs (R-spondin1-4). They share an overall similarity of 40-60% sequence homology and domain architecture. All 4 RSPO family members contain an N-terminal secretory signal peptide, 2 tandem furin-like cysteine-rich (Fu-CRD) domains, a thrombospondin type1 repeat (TSP) domain, and a C-terminal basic amino acid-rich (BR) domain. Each RSPO1, RSPO2, RSPO3 and RSPO4 can serve as ligands for both LGR5 and LGR6 receptors to which they can bind with high affinity. Therefore, certain embodiments of the present disclosure concern the use of polypeptides derived from RSPOs that can be used to target a therapeutic agent to cells that express LGR4, LGR5 or LGR6, such as tumor stem cells that express LGR6. In one particular aspect, the Fu1-Fu2 domains of RSPO1 and/or RSPO2 are linked to a cytotoxic agent, such as the toxin monomethylaurostatin E (MMAE), to selectively target tumors that express high levels of the LGR6.

RSPOs can play a role in the development of cancers and maintenance of stem cells. The small population of stem cells that sustain epithelia throughout the body proliferate and subsequently differentiate in response to growth factors in their niche and their progeny progress through a series of transcriptional states as they differentiate and lose proliferative potential (Clarke, 2019). Signaling in the WNT pathways control fate decisions during embryogenesis and in many adult tissues (Raslan and Yoon, 2019). WNT signaling is regulated by a combination of WNT ligands that bind to various kinds of frizzled receptors, and R-spondins (RSPO) that bind to leucine-rich repeat containing G protein-coupled receptors (LGRs). Cells in the immediate environment of the stem cell niche are the major source of the ligands that drive the WNT signaling and the most effective signaling molecules appear to be transmitted to stem cells over very short distances. RSPO1 has a dominant but no exclusive position among the 4 members of the RSPO family in malignant tissues, and among the members of the LGR family of receptors most evidence points toward LGR5 and LGR6 as having pivotal roles (Yan et al., 2017).

### II. Cytotoxic Agents

A variety of cytotoxic moieties can be included in constructs of the present disclosure. In some embodiments, the cytotoxic moiety is a conjugated drug or a polypeptide.

A variety of conjugated drugs can be used as the cytotoxic moiety. Conjugated drugs that may be used include compound classes such as maytansinoids, auristatins, amanitins, calicheamycins, psymberins, duocarmycins, anthracyclins, camptothecins, doxorubicins, taxols, and pyrrolobenzodiazepines. Specific examples of cytotoxic agents include paclitaxel, docetaxel, etoposide, tenoposide, vincristine, vinblastine, colchicine, doxorubicin, daunorubicin, mithramycin, actinomycin, glucorticoids, puromycin, epirubicin, cyclophosphamide, methotrexate, cytarabine, f-fluorouracil, platins, streptozotocin, minomycin C, anthracyclines, dactinomycin or actinomycin, bleomycin, mithramycin, anthramycin, duocarmycins, ifosfamide, mitoxantrone, daunomycin, carminomycin, animoterin, melphalan, esperamicins, lexitropsins, auristatins (e.g., auristatin E, auristatin F, AEB, AEVB, AEFP, MMAE, MMAF), eleuthorobin, netropsin, podophyllotoxins, maytansiods including maytansine and DM1, deruxtecan and combretestatins.

In some embodiments, the conjugated drug is Monomethyl auristatin E (MMAE, also called vedotin). MMAE is a potent antimitotic agent that can inhibit cell division by blocking tubulin polymerization. Auristatins are synthetic analogues of the antineoplastic natural product Dolastatin, and auristatins have previously been used as payloads in antibody-drug conjugates. MMAE is 100-1000 times more potent than doxorubicin (Adriamycin/Rubex).

In some embodiments, the cytotoxic moiety is a serine protease, such as granzyme B (GrB). The GrB may include a variety of mutations, such as those described in U.S. Pat. No. 9,096,840 or U.S. Patent Application Nos. 2014/0140976 and 2015/0010556. For example, in some aspects, the recombinant serine protease is a GrB polypeptide and comprises the sequence YVDEVDIIGGHEAK (SEQ ID NO:21); RVRRIIGGHEAK (SEQ ID NO:22); RVRRIIGGHEAK (SEQ ID NO:23); (I/A)(E/D)GRIIGGHEAK (SEQ ID NO:24); YEVDIIGGHEAK (SEQ ID NO:25); WEHDIIGGHEAK (SEQ ID NO:26); DVADIIGGHEAK (SEQ ID NO:27); DEHDIIGGHEAK (SEQ ID NO:28); DEVDIIGGHEAK (SEQ ID NO:29); DMQDIIGGHEAK (SEQ ID NO:30); LEVDIIGGHEAK (SEQ ID NO:31); LEHDIIGGHEAK (SEQ ID NO:32); VEIDIIGGHEAK (SEQ ID NO:33); VEHDIIGGHEAK (SEQ ID NO:34); IETDIIGGHEAK (SEQ ID NO:35); LETDIIGGHEAK (SEQ ID NO:36) or IEADIIGGHEAK (SEQ ID NO:37). Other serine proteases that can be utilized include Cathepsin G (NCBI accession no. P08311), Chymase (NCBI accession no. P23946), Myeloblastin (NCBI accession no. P24158), Kallikrein-14 (NCBI accession no. Q9P0G3), Complement factor D (NCBI accession no. K7ERG9), PRSS3 protein (NCBI accession no. A1A508), Trypsin-1 (NCBI accession no. P07477), Serine protease 57 (NCBI accession no. Q6UWY2) and PRSSL1 protein (NCBI accession no. B7ZMF6), or a polypeptide having at least 90% or at least 95% sequence identity.

In some embodiments, the cytotoxic moiety is a cytotoxic protein. Cytotoxic proteins that can be used include apoptotic factors or apoptosis related proteins including AIF, Apaf (e.g., Apaf-1, Apaf-2, Apaf-3), oder APO-2 (L), APO-3 (L), Apopain, Bad, Bak, Bax, Bcl-2, Bcl-x_{L}, Bcl-x_{S}, bik, CAD, Calpain, Caspase e.g. Caspase-1, Caspase-2, Caspase-3, Caspase-4, Caspase-5, Caspase-6, Caspase-7, Caspase-8, Caspase-9, Caspase-10, Caspase-11, ced-3, ced-9, c-Jun, c-Myc, crm A, cytochrom C, CdR1, DcR1, DD, DED, DISC, DNA-PKcs, DR3, DR4, DR5, FADD/MORT-1, FAK, Fas (Fas-ligand CD95/fas (receptor)), FLICE/MACH, FLIP, fodrin, fos, G-Actin, Gas-2, gelsolin, granzyme A/B, ICAD, ICE, JNK, lamin A/B, MAP, MCL-1, Mdm-2, MEKK-1, MORT-1, NEDD, NF-ₖₐₚₚₐB, NuMa, p53, PAK-2, PARP, perforin, PITSLRE, PKCdelta, pRb, presenilin, prICE, RAIDD, Ras, RIP, sphingomyelinase, thymidinkinase from herpes simplex, TRADD, TRAF2, TRAIL-R1, TRAIL-R2, TRAIL-R3, and transglutaminase.

In further embodiments, the cytotoxic agent may be selected from bispecific antibodies and bioactive compounds including nucleic acids like DNA, mRNA, siRNA, and fragments of these; pharmaceutical compounds such as various therapeutic drugs; and radionuclides and cytotoxins, which can be targeted to a desired tissue or cell by the targeting moiety. These agents may act while they remain conjugated to the targeting protein or a portion thereof, or they may first detach from the targeting protein if the linking group is one that can readily cleave in vivo.

Suitable cytotoxic agents for use with the present disclosure include microtubule inhibitors, topoisomerase I inhibitors (e.g., deruxtecan), intercalating agents, inhibitors of intracellular signaling pathways, kinase inhibitors, transcription inhibitors such as siRNAs, aRNAs, and miRNAs, and DNA minor groove binders. Select cytotoxic agents that can be used in select embodiments of the present disclosure include calicheamicin, MMAE, DM1, deruxtecan, SN-38, MMAF, PE38, diphtheria toxin, and 90-yttrium. Select cytotoxic agents that can be used in various embodiments are also discussed in Kostova et al. (2021), Chen et al. (2017), and Lambert et al. (2017).

### A. Maytansinoids

Maytansinoids (also called maytansine analogs) are semi-synthetic agents derived from the natural product maytansine. Maytansinoids include the emtansine, which can disrupt microtubule function. Maytansine can be obtained via fermentation, and the molecule can be synthetically modified to result in maytansinoids DM1, DM3, and DM4, as shown below. The different side chains on maytansinoids can give different release and stability profiles. Maytansinoids include the following compounds:

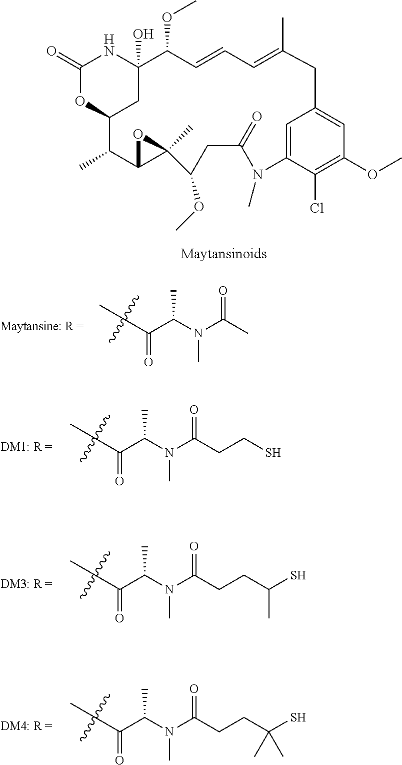

Maytansinoids that can be used in various embodiments are further discussed, e.g., in Chen et al. (2017).

### B. Auristatins

A variety of auristatins can be used as the cytotoxic moiety in various embodiments of the present disclosure. Auristatins are typically synthetic compounds that share some structural similarity with the natural compound dolastatin 10. Select auristatins that can be used in embodiments of the present disclosure include the following:

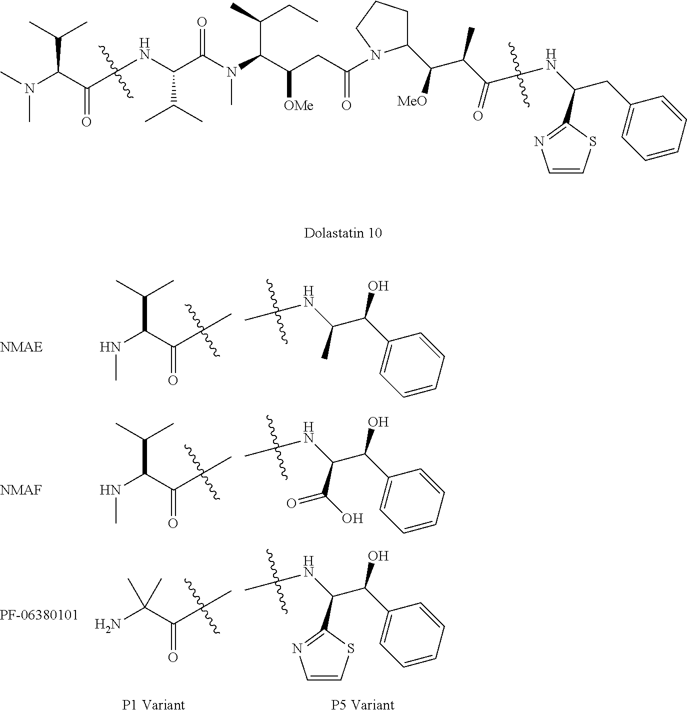

Select auristatins that can be used in various embodiments are reviewed, e.g., in Kostova et al. (2021). In select embodiments, the cytotoxic payload is MMAE, MMAF, or PF-06380101.

### C. Tubulysins

The cytotoxic moiety may be a Tubulysin payload. Tubulysin-based payloads may be derived from naturally occurring tubulysins (e.g., tubulysin A, B, C), and typically function as microtubule destabilizing agents. In some embodiments the cytotoxic moiety is Tubulysin A, Tubulysin B, Tubulysin C, Tubulysin G, or tubulysin I.

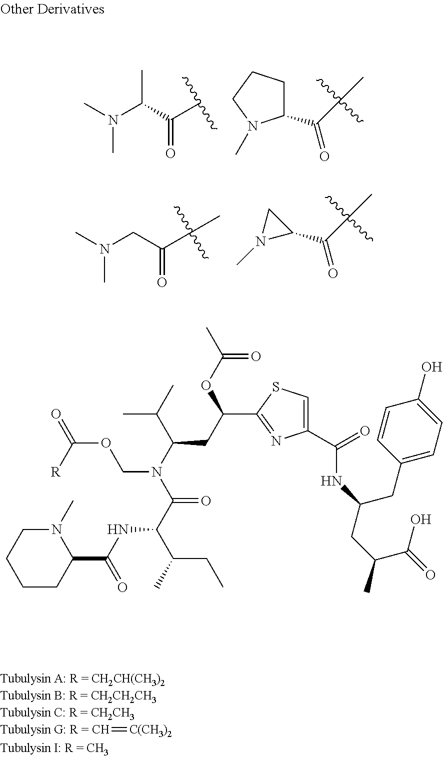

### D. Eribulin

The cytotoxic moiety may be eribulin. Eribulin is a tubulin disrupting agent and has the structure:

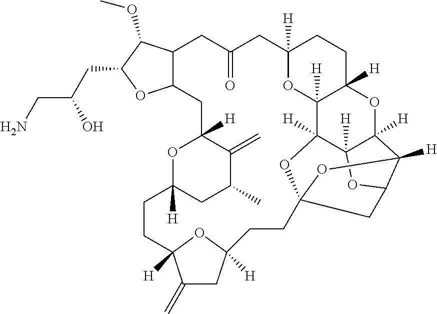 [SMILES: C=C1C[C@@H]2CC[C@@]34CC5O[C@H]6[C@@H](O3)[C@H]3O[C@H](CC[C@@H]3O[C@H]6[C@H]5O4)CC(=O)CC3C(C[C@H]4O[C@@H](CC[C@@H]1O2)C[C@@H](C)C4=C)O[C@H](C[C@H](O)CN)[C@@H]3OC]

### E. Taxol Derivatives

The cytotoxic moiety may be a taxol derivative. Taxol derivatives can inhibit cell growth by stabilizing microtubule filaments. Taxol derivatives include docetaxel and paclitaxel.

### F. DNA Damaging Agents

In some embodiments, the cytotoxic moiety is calicheamicin. Calicheamicin is an antitumor-antibiotic that may cause double-strand breakage of DNA. Calicheamicin has the structure:

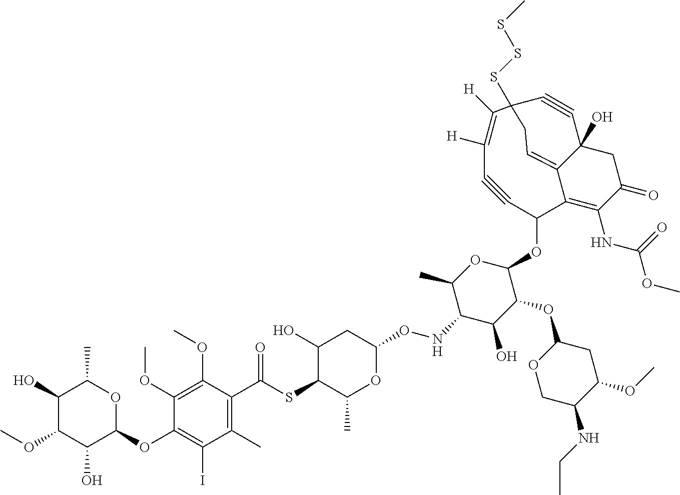

Additional DNA damaging agents that may be used include anthramycin-based dimers and duocarmycin, anthracycline, and camptothecin.

### III. Linkers

A variety of linkers can be used to attach a cytotoxic moiety to a cell targeting moiety (e.g., a R-spondin targeting moiety) in various embodiments of the present disclosure. Non-limiting examples of linkers that may be used are provided in Table 1, below.

TABLE 1
Example Linkers

| Abbreviation | Chemical Structure | Linker Type |
|---|---|---|
| MHH | 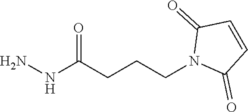 [SMILES: NNC(=O)CCCN1C(=O)C=CC1=O] | Chemical labile (acid labile) linker |
| DSDM | 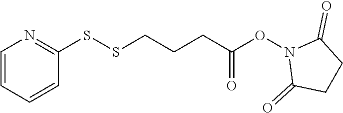 [SMILES: O=C(CCCSSc1ccccn1)ON1C(=O)CCC1=O] | Disulfide- containing reducible linker |
| Sulfo-SPDB | 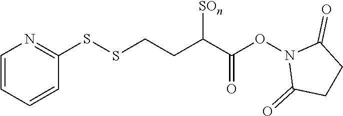 | Disulfide- containing reducible linker |
| MC-VC- PABC | 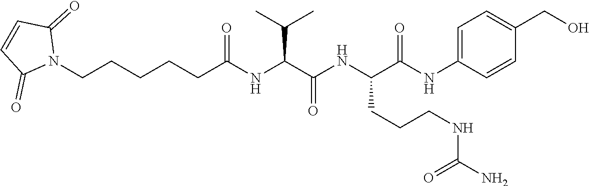 [SMILES: CC(C)[C@H](NC(=O)CCCCCN1C(=O)C=CC1=O)C(=O)N[C@@H](CCCNC(N)=O)C(=O)Nc1ccc(CO)cc1] | Enzymatically cleavable linker |
| SMCC | 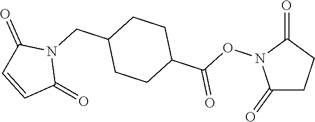 [SMILES: O=C(ON1C(=O)CCC1=O)C1CCC(CN2C(=O)C=CC2=O)CC1] | Non-cleavable bifunctional linker |
| Mal-PEG- NHS | 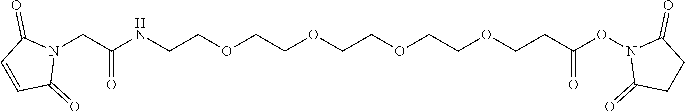 [SMILES: O=C(CN1C(=O)C=CC1=O)NCCOCCOCCOCCOCCC(=O)ON1C(=O)CCC1=O] | Non-cleavable spacer linker |
| GBC | 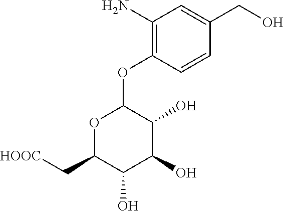 [SMILES: Nc1cc(CO)ccc1OC1O[C@H](CC(=O)O)[C@@H](O)[C@H](O)[C@H]1O] | Enzyme-labile β-Glucuronide linker |

Select linkers that can be used in various embodiments of the present disclosure are also discussed in Kostova et al. (2021), Chen et al. (2017), and Lambert et al. (2017). In some embodiments, the linker is MHH, DSDM, Sulfo-SPDB, MC-VC-PABC, SMCC, Mal-PEG-NHS, GGFG, or GBC.

In some embodiments, a peptide linker is included in a construct of the present disclosure near or adjacent to a sortase recognition sequence as described herein. The linker (e.g., a G₄S linker) may promote or facilitate conformational freedom of the sortase domain to recognize a sortase recognition motif. In some embodiments, the linker is a (GGS), (GGGS), or (G₄S) linker. The linker can be repeated, e.g., 1, 2, 3, 4, 5, 6, 7, or 8 times (e.g., (GₓS)ₙ, wherein x=1-4 and n=1-9), more preferably repeated 1 or two times (e.g., (GₓS)ₙ, wherein x=1-4 and n=1-3).

### IV. Mutant Fc Region

In some embodiments, a construct of the present disclosure may contain a wild-type or mutant Fc region. For example, the construct may contain a cell-targeting moiety (e.g., a R-spondin targeting moiety), a cytotoxic moiety (e.g., MMAE or MMAF), and a mutant Fc region (e.g., DHS Fc). The Fc region may cause dimerization of the construct. In select embodiments, it has been observed that inclusion of the DHS Fc region in a construct can improve stability, potency, and/or half-life of the construct.

A “DHS Fc” region, as used herein, refers to a polypeptide comprising a variant human IgG Fc domain that can bind human FcRn at an acidic pH, wherein the Fc domain has the following substitutions: (i) aspartic acid at position 309 (L/V309D); (ii) histidine at position 311 (Q311H); and (iii) a substitution at position 434 of either a serine or tyrosine (N434Y or N434S); with amino acid position numbering being according to the Kabat system. The DHS Fc region may optionally further comprise the substitution mutation V264E (e.g., mutations L/V309D, Q311H, N434S/Y; and optionally, V264E), wherein the DHS Fc region can bind human FcRn at an acidic pH. Without wishing to be bound by any theory, inclusion of the of the DHS Fc region in a construct of the present disclosure may increase the half-life due to binding of the human FcRn. DHS mutants are disclosed in Lee et al. (2019) and U.S. patent Ser. No. 11/059,892, which are incorporated by reference in their entirety, and can be included in constructs of the present disclosure.

In some embodiments, mutant or variant human Fc domains are provided that, as compared to a corresponding wild-type Fc domain, exhibit: (i) enhanced binding at pH 5.8 and (ii) reduced binding or no detectable binding at pH 7.4 for FcRn. The mutant or variant Fc domain may be a mutant or variant IgG domain. The mutant or variant Fc domain may be comprised in a polypeptide, such as an antibody. In some embodiments, the mutant or variant Fc domain may be comprised in a therapeutic antibody such as, e.g., an agonistic or antagonistic antibody. In some embodiments, there are compositions involving a polypeptide that has a mutant or variant Fc domain derived from a human IgG1-4 antibody (“antibody Fc domain”). The mutant Fc domain may be a variant of the wild-type human IgG1 Fc domain (SEQ ID NO:9), wherein the mutant or variant Fc domain enables binding to FcRn with increased affinity at acidic pH and not at neutral pH. In some embodiments, the engineered Fc domain may display increased affinity for FcRn of, e.g., of about 5-fold greater than a glycosylated wild-type Fc domain. In further embodiments, mutant human Fc domains are provided of all the other wild-type IgG subclasses (human IgG2, IgG3, and IgG4) are provided and enable binding to FcRn with increased affinity at acidic pH and not at neutral pH. The mutant or variant Fc domain may contain the mutations (L/V309D, Q311H, N434S/Y), and optionally (V264E), relative to a wild-type human IgG1 Fc (SEQ ID NO:1), human IgG2 Fc (SEQ ID NO:10), human IgG3 Fc (SEQ ID NO:11), or a human IgG4 Fc (SEQ ID NO:12), in order to increase the binding of the mutant or variant Fc to FcRn at acidic pH (e.g., pH 5.8), but not at physiological pH (pH 7.4). For example, mutations (L/V309D, Q311H, N434S) were made in human IgG1, IgG2, IgG3, and IgG4, resulting in SEQ ID NO:13, SEQ ID NO:14, SEQ ID NO:15, and SEQ ID NO:16, respectively.

As used herein, a protein or peptide generally refers, but is not limited to, a protein of greater than about 200 amino acids, up to a full-length sequence translated from a gene; a polypeptide of greater than about 100 amino acids; and/or a peptide of from about 3 to about 100 amino acids. For convenience, the terms “protein,” “polypeptide,” and “peptide” are used interchangeably herein.

As used herein, an “amino acid residue” refers to any amino acid, amino acid derivative, or amino acid mimic as would be known to one of ordinary skill in the art. In certain embodiments, the residues of the proteinaceous molecule are sequential, without any non-amino acid residue interrupting the sequence of amino acid residues. In other embodiments, the sequence may comprise one or more non-amino acid moieties. In particular embodiments, the sequence of residues of the proteinaceous molecule may be interrupted by one or more non-amino acid moieties.

As used herein a “distinct Fc domain” may be defined as a domain that differs from another Fc by as little as one amino acid. Methods for making a library of distinct antibody Fc domains or nucleic acids that encode antibodies are well known in the art. For example, in some cases Fc domains may be amplified by error prone PCR. Furthermore, in certain cases a plurality of antibody Fc domains may comprise a stretch (1, 2, 3, 4, 5, 6, 7, 8, 9, 10 or more) of amino acids that have been randomized. In certain cases, specific mutations may be engineered into Fc domains. For example, in some aspects, residues that are normally glycosylated in an antibody Fc domain may be mutated. Furthermore, in certain aspects, residues that are normally glycosylated (or adjacent residues) may be used as a site for an insertion of 1, 2, 3, 4, 5, 6, 7, 8, 9, 10 or more amino acids.

A polypeptide may comprise a mutant or variant antibody Fc domain capable of binding an FcR polypeptide. In some aspects, the Fc domain may be further defined as having a specific affinity for an FcR polypeptide under physiological conditions. For instance an Fc domain may have an equilibrium dissociation constant between about 10-6 M to about 10-9 M under physiological conditions. Furthermore in some aspects an aglycosylated Fc domain may be defined as comprising one or more amino acid substitutions or insertions relative to a wild-type sequence, such as a human wild-type sequence. The Fc domain may be glycosylated or aglycosylated.

Means of preparing such a polypeptide include those discussed in PCT Publn. WO 2008/137475, which is hereby incorporated by reference. One can alternatively prepare such polypeptides directly by genetic engineering techniques such as, for example, by introducing selected amino acid substitutions or insertions into a known Fc background, wherein the insertion or substitution provides an improved FcR binding capability to aglycosylated Fc regions, as discussed above. In some embodiments, an Fc domain is engineered to bind one or more specific Fc receptors. Additionally or alternatively, an Fc domain may be engineered so that it does not specifically bind one or more specific Fc receptors.

In some embodiments, an Fc domain comprises a specific binding affinity for an FcR such as human FcγRIA, FcγRIIA, FcγRIIB, FcγRIIc, FcγRIIIA, FcγRIIIb, FcαRI, or for C1q. In some embodiments, the antibody or polypeptide containing the Fc domain is glycosylated and displays FcR binding that is similar to, essentially the same as, or the same as FcR binding for the wild type antibody, e.g., as compared to the corresponding IgG2, IgG3, or IgG4 antibody. In some embodiments, the antibody is glycosylated. In some embodiments, the antibody or polypeptide containing the Fc domain is aglycosylated. The binding affinity of an antibody Fc or other binding protein can, for example, be determined by the Scatchard analysis of Munson and Pollard (1980). Alternatively, binding affinity can be determined by surface plasmon resonance or any other well-known method for determining the kinetics and equilibrium constants for protein:protein interactions. Isolated IgG variants are provided below in Table 1. In various embodiments, the mutations may be introduced into an IgG1 Fc domain (e.g., resulting in SEQ ID NO: 13), or corresponding mutations may be made in an IgG2 Fc domain (e.g., SEQ ID NO:10), an IgG3 Fc domain (e.g., SEQ ID NO:11), or an IgG4 Fc domain (e.g., SEQ ID NO:12), as desired.

TABLE 1
IgG variants (Sequence numbering is based
on Kabat and mutations are specified below)

EDHS (V264E, L309D, Q311H, N434S; SEQ ID NO: 38),
EDHY (V264E, L309D, Q311H, N434Y; SEQ ID NO: 39),
DHS (L309D, Q311H, N434S; SEQ ID NO: 13),
DHY (L309D, Q311H, N434Y; SEQ ID NO: 40),
IgG2-DHS (V309D, Q311H, N434S; SEQ ID NO: 14),
IgG3-DHS (L309D, Q311H, N434S; SEQ ID NO: 15),
IgG4-DHS (L309D, Q311H, N434S; SEQ ID NO: 16)

By “position” as used herein is meant a location in the sequence of a protein. Positions may be numbered sequentially, or according to an established format, for example the EU index for antibody numbering.

For all positions discussed in the present invention, numbering is according to the EU index. The “EU index” or “EU index as in Kabat” or “EU numbering scheme” refers to the numbering of the EU antibody (Edelman et al., 1969; Kabat et al., 1991; both incorporated herein by reference in their entirety).

In certain embodiments the size of the at least one Fc polypeptide proteinaceous molecule may comprise, but is not limited to, at least 15, 16, 17, 18, 19, 20, 21, 22, 23, 24, 25, 30, 35, 40, 45, 50, 55, 60, 65, 70, 75, 80, 85, 90, 95, 100, 110, 120, 130, 140, 150, 160, 170, 180, 190, 200, 210, 220, 225 or greater amino molecule residues, and any range derivable therein. Compounds may include the above-mentioned number of contiguous amino acids from SEQ ID NO:13-16 (human IgG1-4 Fc polypeptide) or from SEQ ID NOs: 13-16, and these may be further qualified as having a percent sequence identity or homology to a wild-type human IgG Fc domain (e.g., percent sequence identity to any one of SEQ ID NOs:1-4).

### V. Sortase Recognition Sequence

In some embodiments, a sortase recognition sequence is included in a construct of the present disclosure. For example, the sortase recognition sequence may be included in order to attach a cytotoxic moiety to a polypeptide comprising a cell-targeting moiety (e.g., a R-spondin targeting polypeptide such as FuFu). Sortase-catalyzed transacylation reactions can allow the preparation of head-to-tail protein-protein fusions, with high specificity and in near-quantitative yields (e.g., Popp et al. (2011), Guimaraes et al. (2011), Popp et al. (2007)).

Sortases, sortase-mediated transacylation reactions, and their use in transacylation (sometimes also referred to as transpeptidation) for protein engineering are well known to those of skill in the art (see, e.g., International Patent Application PCT/US2010/000274, and International Patent Application PCT/US2011/033303). The transpeptidation reaction catalyzed by sortase can be used to ligate a polypeptide comprising a transamidase recognition motif with those bearing one or more N-terminal glycine residues. In some embodiments, the sortase recognition motif is an LPXT motif or an LPXT(G)ₙ motif. As is known in the art, the substitution of the C-terminal residue of the recognition sequence with a moiety exhibiting poor nucleophilicity once released from the sortase can result in a more efficient ligation.

Sortase-mediated transacylation reactions are catalyzed by the transamidase activity of sortase. A transamidase is an enzyme that can form a peptide linkage (i.e., amide linkage) between an acyl donor compound and a nucleophilic acyl acceptor containing a NH₂—CH₂-moiety. In some embodiments, the sortase is sortase A (SrtA) or sortase E. However, any sortase or transamidase catalyzing a transacylation reaction can be used in embodiments of the present disclosure.

In some embodiments, the sortase recognition sequence is LPXT (SEQ ID NO:41), wherein X is a standard or non-standard amino acid. In some embodiments, X is selected from D, E, A, N, Q, K, or R. For example, in some embodiments the recognition sequence is LPET (SEQ ID NO:42). In some embodiments, the recognition sequence is selected from LPXT (SEQ ID NO:41), SPXT (SEQ ID NO:42), LAXT (SEQ ID NO:43), LSXT (SEQ ID NO:44), NPXT (SEQ ID NO:45), VPXT (SEQ ID NO:46), IPXT (SEQ ID NO:47), and YPXR (SEQ ID NO:48). In some embodiments, X is selected to match a naturally occurring transamidase recognition sequence. In some embodiments, sortase recognition sequences described in PCT international patent application WO 2013003555, U.S. Pat. No. 7,238,489 and U.S. Patent Application 2014/0030697 can be used. The sortase recognition sequence may be, e.g., LPKTG (SEQ ID NO:49), LPATG (SEQ ID NO:50), LPNTG (SEQ ID NO:51), LPETG (SEQ ID NO:52), LPXAG (SEQ ID NO:53), LPNAG (SEQ ID NO:54), LPXTA (SEQ ID NO:55), LPNTA (SEQ ID NO:56), LGXTG (SEQ ID NO:57), LGATG (SEQ ID NO:58), IPXTG (SEQ ID NO:59), IPNTG (SEQ ID NO:60), IPETG (SEQ ID NO:61), LPKTGG (SEQ ID NO:62), LPATGG (SEQ ID NO:63), LPNTGG (SEQ ID NO:64), LPETGG (SEQ ID NO:65), LPXAGG (SEQ ID NO:66), LPNAGG (SEQ ID NO:67), LPXTAG (SEQ ID NO:68), LPNTAG (SEQ ID NO:69), LGXTGG (SEQ ID NO:70), LGATGG (SEQ ID NO:71), IPXTGG (SEQ ID NO:72), IPNTGG (SEQ ID NO:73), and IPETGG (SEQ ID NO:74).

Sortase E can be used to covalently attach a cytotoxic moiety to a polypeptide comprising an LGR binding domain. Sortase E can recognize the sequence LAHTGG (SEQ ID NO: 106). For this reason, sortase A and sortase E can be separately used to covalently attach cytotoxic moieties to both the C-terminal and N-terminal sides of the polypeptide. The cytotoxic moieties can have the same structure (e.g., both are MMAE) or different cytotoxic moieties can be covalently bound to the polypeptide. Using both sortase A and sortase E can confer advantages for production. For example, In the case of FcF2-LPETGG-His, the sortase reaction can to put MMAE warheads on both ends of the molecule; however, a challenge is that the sortase A reaction is reversible so that, in the process of adding a second MMAE to the N-terminal end, it might remove and MMAE already loaded on the C-terminal end. The recently isolated sortase E can be used to link substrates containing an N-terminal GG motif to the sequence LAHTGG (SEQ ID NO: 106) on the C-terminal end of other proteins or peptides. Since sortase A and sortase E have high specificity for different recognition sequences (LPETGGG versus LAHTGG), they can simultaneously or separately be used to covalently attach a cytotoxic moiety (e.g., MMAE, etc.) on both ends of the polypeptide (containing the LGR binding moiety) that contains a diglycine at the N-terminal end and a LPXT (SEQ ID NO:41) (preferably LPETGG, SEQ ID NO: 65) sequence at the C-terminal end.

In some embodiments, the coding sequence of sortase recognition is operably linked to the coding sequence of the serine protease via a linker. Any suitable linker known to one of skilled in the art can be used. In a particular embodiment, the linker is a (GGS), (GGGS; SEQ ID NO:75), or (G₄S) linker. In some embodiments, the (G₄S) linker can facilitate conformational freedom of the sortase domain to recognize the sortase recognition motif.

### VI. Partial Reduction of Disulfides

If desired, a partial reduction of disulfides can be used to attach a cytotoxic moiety to a polypeptide containing the LGR binding domain. Polypeptides provided herein may contain disulfides in either the Fc hinge region (e.g., 3 disulfide bonds in a immunoglobulin Fc domain) and/or the LGR binding domain (e.g., 8 in the Fu1-Fu2 domains) that can be targeted. Covalent attachment of cytotoxin(s) by partial reduction of Fc domain disulfide bonds or disulfide bonds in the LGR binding domain, followed by reaction with a linker containing a thiol-reactive maleimide group previously coupled to the cytotoxin can be used. These methods of loading can be used to attach 2, 3, 4, 5, 6, 7, 8 or more cytotoxins per molecule, but should be individualized as excess loading can distort protein structure and reduce plasma half-lives. For example, the polypeptide (e.g., FcF2-His) can be exposed to TCEP at concentrations from 0.0005 to 5000 PM, and subsequently (e.g., about 25 min later) a cytotoxin comprising maleimide (e.g., maleimide-val/cit-PAB-MMAE) can be added to the reaction (e.g., at ratios of about 1:1-1:9, 1:2, 1:8 (protein:cytotoxic agent)). The reaction may then be allowed to continue for a sufficient duration to achieve binding of the cytotoxic agent. In some preferred embodiments, the cytotoxic agent comprises a cleavable linker (e.g., comprising valine (Val)-citrulline (Cit) bond).

### VII. Radiotherapies and Imaging Agents

In some embodiments, a therapeutic compound provided herein may comprise a radiotherapy or an imaging agent. For example, the therapeutic compound may be covalently bonded to or conjugated to a radioisotope such as, e.g., iodine-131, strontium-89, samarium-153, or radium-223. A polypeptide described herein (e.g., SEQ ID NOs: 1-4, 19, or 20) may be covalently bonded to or conjugated to an imaging agent or a contrast agent. The imaging agent may be, e.g., an iodinated contrast media, an ionic iodinated contrast media, an MRI contrast agent (e.g., gadolinium), a diagnostic dye, a non-iodinated contrast media, a non-ionic iodinated contrast media, or an ultrasound contrast media. Additional radiotherapies and imaging agents that can be covalently bonded to or conjugated with a compound or polypeptide described herein include, e.g., lutetium-177.

### VIII. Modified Proteins and Polypeptides

Some embodiments concern modified proteins and polypeptides, particularly a modified protein or polypeptide that exhibits at least one functional activity that is comparable to the unmodified version, yet the modified protein or polypeptide possesses an additional advantage over the unmodified version, such as suppressing B-cell activation, being easier or cheaper to produce, eliciting fewer side effects, and/or having better or longer efficacy or bioavailability. Thus, when the present application refers to the function or activity of “modified protein” or a “modified polypeptide” one of ordinary skill in the art would understand that this includes, for example, a protein or polypeptide that 1) performs at least one of the same activities or has at least one of the same specificities as the unmodified protein or polypeptide, but that may have a different level of another activity or specificity; and 2) possesses an additional advantage over the unmodified protein or polypeptide. Determination of activity may be achieved using assays familiar to those of skill in the art, particularly with respect to the protein's activity, and may include for comparison purposes, for example, the use of native and/or recombinant versions of either the modified or unmodified protein or polypeptide. It is specifically contemplated that embodiments concerning a “modified protein” may be implemented with respect to a “modified polypeptide,” and vice versa. In addition to the modified proteins and polypeptides discussed herein, embodiments may involve domains, polypeptides, and proteins described in PCT Publn. WO 2008/137475, which is hereby specifically incorporated by reference.

Modified proteins may possess deletions and/or substitutions of amino acids; thus, a protein with a deletion, a protein with a substitution, and a protein with a deletion and a substitution are modified proteins. In some embodiments these modified proteins may further include insertions or added amino acids, such as with fusion proteins or proteins with linkers, for example. This may include the insertion of a targeting peptide or polypeptide or simply a single residue. Terminal additions, called fusion proteins, are discussed below.

A “modified deleted protein” lacks one or more residues of the native protein, but possesses the specificity and/or activity of the native protein. A “modified deleted protein” may also have reduced immunogenicity or antigenicity. An example of a modified deleted protein is one that has an amino acid residue deleted from at least one antigenic region (i.e., a region of the protein determined to be antigenic in a particular organism, such as the type of organism that may be administered the modified protein).

Substitutional or replacement variants typically contain the exchange of one amino acid for another at one or more sites within the protein and may be designed to modulate one or more properties of the polypeptide, particularly its effector functions and/or bioavailability. Substitutions may or may not be conservative, that is, one amino acid is replaced with one of similar shape and charge. Conservative substitutions are well known in the art and include, for example, the changes of: alanine to serine; arginine to lysine; asparagine to glutamine or histidine; aspartate to glutamate; cysteine to serine; glutamine to asparagine; glutamate to aspartate; glycine to proline; histidine to asparagine or glutamine; isoleucine to leucine or valine; leucine to valine or isoleucine; lysine to arginine; methionine to leucine or isoleucine; phenylalanine to tyrosine, leucine, or methionine; serine to threonine; threonine to serine; tryptophan to tyrosine; tyrosine to tryptophan or phenylalanine; and valine to isoleucine or leucine.

It also will be understood that amino acid and nucleic acid sequences may include additional residues, such as additional N- or C-terminal amino acids or 5′ or 3′ sequences, and yet still be essentially as set forth in one of the sequences disclosed herein, so long as the sequence meets the criteria set forth above, including the maintenance of biological protein activity where protein expression is concerned. The addition of terminal sequences particularly applies to nucleic acid sequences that may, for example, include various non-coding sequences flanking either of the 5′ or 3′ portions of the coding region or may include various internal sequences, i.e., introns, which are known to occur within genes.

A modified polypeptide may be characterized as having a certain percentage of identity to an unmodified polypeptide or to any polypeptide sequence (e.g., SEQ ID NOs: 1-4, 19, or 20) described herein. The percentage identity may at least about 70%, 75%, 80%, 85%, 90%, 95%, 96%, 97%, 98%, 99%, 99.5% or 100% identity, or any range derivable therein, between the modified polypeptide and the unmodified polypeptide. It is contemplated that percentage of identity discussed above may relate to a particular region of a polypeptide compared to an unmodified region of a polypeptide.

In making such changes, the hydropathic index of amino acids may be considered. The importance of the hydropathic amino acid index in conferring interactive biologic function on a protein is generally understood in the art. It is accepted that the relative hydropathic character of the amino acid contributes to the secondary structure of the resultant protein, which in turn defines the interaction of the protein with other molecules, for example, enzymes, substrates, receptors, DNA, antibodies, antigens, and the like.

It also is understood in the art that the substitution of like amino acids can be made effectively on the basis of hydrophilicity. U.S. Pat. No. 4,554,101 states that the greatest local average hydrophilicity of a protein, as governed by the hydrophilicity of its adjacent amino acids, correlates with a biological property of the protein. As detailed in U.S. Pat. No. 4,554,101, the following hydrophilicity values have been assigned to amino acid residues: arginine (+3.0); lysine (+3.0); aspartate (+3.0±1); glutamate (+3.0±1); serine (+0.3); asparagine (+0.2); glutamine (+0.2); glycine (0); threonine (−0.4); proline (−0.5±1); alanine (−0.5); histidine (−0.5); cysteine (−1.0); methionine (−1.3); valine (−1.5); leucine (−1.8); isoleucine (−1.8); tyrosine (−2.3); phenylalanine (−2.5); tryptophan (−3.4). It is understood that an amino acid can be substituted for another having a similar hydrophilicity value and still produce a biologically equivalent and immunologically equivalent protein. In such changes, the substitution of amino acids whose hydrophilicity values are within ±2 is preferred, those that are within ±1 are particularly preferred, and those within ±0.5 are even more particularly preferred.

As outlined above, amino acid substitutions generally are based on the relative similarity of the amino acid side-chain substituents, for example, their hydrophobicity, hydrophilicity, charge, size, and the like. Exemplary substitutions that take into consideration the various foregoing characteristics are well known to those of skill in the art and include: arginine and lysine; glutamate and aspartate; serine and threonine; glutamine and asparagine; and valine, leucine, and isoleucine.

In some aspects, it has been observed herein that glycosylation can significantly affect yield and pharmacology of polypeptides provided herein. For example, changes in glycosylation such as caused by mutation or substitution at an asparagine can result in changes in protein folding, stability, pharmacokinetics and other features of its pharmacology. As would be appreciated by one of skill in the art, different amounts of glycosylation can be achieved either by including substitution mutations in the polypeptide (e.g., alanine for asparagine) or by using different types of cells, such as yeast, insect, human, or bacterial cells, for producing the polypeptides provided herein.

### IX. Pharmaceutical Preparations

Pharmaceutical compositions of the present embodiments comprise an effective amount of one or more the present compounds and at least one additional agent dissolved or dispersed in a pharmaceutically acceptable carrier. The phrases “pharmaceutical or pharmacologically acceptable” refers to molecular entities and compositions that do not produce an adverse, allergic or other untoward reaction when administered to an animal, such as, for example, a human, as appropriate. The preparation of a pharmaceutical composition that contains at least one chimeric polypeptide or additional active ingredient will be known to those of skill in the art in light of the present disclosure, as exemplified by Remington: the Science and Practice of Pharmacy (23^{rd} edition, Elsevier, 2020). Moreover, for animal (e.g., human) administration, it will be understood that preparations should meet sterility, pyrogenicity, general safety and purity standards as required by FDA Office of Biological Standards.

As used herein, “pharmaceutically acceptable carrier” includes any and all solvents, dispersion media, coatings, surfactants, antioxidants, preservatives (e.g., antibacterial agents, antifungal agents), isotonic agents, absorption delaying agents, salts, preservatives, drugs, drug stabilizers, gels, binders, excipients, disintegration agents, lubricants, sweetening agents, flavoring agents, dyes, such like materials and combinations thereof, as would be known to one of ordinary skill in the art (see, for example, Remington's Pharmaceutical Sciences, 18th Ed. Mack Printing Company, 1990, pp. 1289-1329, incorporated herein by reference). Except insofar as any conventional carrier is incompatible with the active ingredient, its use in the therapeutic or pharmaceutical compositions is contemplated.

The cell targeted cytotoxic agent may comprise different types of carriers depending on whether it is to be administered in solid, liquid or aerosol form, and whether it needs to be sterile for such routes of administration as injection. The present therapies of the embodiments can be administered intravenously, intradermally, intraarterially, intraperitoneally, intralesionally, intracranially, intraarticularly, intraprostaticaly, intrapleurally, intratracheally, intranasally, intravitreally, intravaginally, intrarectally, topically, intratumorally, intramuscularly, intraperitoneally, subcutaneously, subconjunctival, intravesicularlly, mucosally, intrapericardially, intraumbilically, intraocularally, orally, topically, locally, inhalation (e.g., aerosol inhalation), injection, infusion, continuous infusion, localized perfusion bathing target cells directly, via a catheter, via a lavage, in cremes, in lipid compositions (e.g., liposomes), or by other method or any combination of the forgoing as would be known to one of ordinary skill in the art (see, for example, Remington's Pharmaceutical Sciences, 18th Ed. Mack Printing Company, 1990, incorporated herein by reference).

The actual dosage amount of a composition of the present embodiments administered to an animal patient can be determined by physical and physiological factors such as body weight, severity of condition, the type of disease being treated, previous or concurrent therapeutic interventions, idiopathy of the patient and on the route of administration. The practitioner responsible for administration will, in any event, determine the concentration of active ingredient(s) in a composition and appropriate dose(s) for the individual subject.

In certain embodiments, pharmaceutical compositions may comprise, for example, at least about 0.1% of an active compound, such as a therapeutic compound provided herein (e.g., FcF2-MMAE). The active compound may comprise between about 2% to about 75% of the weight of the unit, or between about 25% to about 60%, for example, and any range derivable therein. As shown the below examples, sub-nanomolar potencies were observed in vitro for select therapeutic compounds (e.g., FcF₂-MMAE), and in vivo activity was detected at a dose of just 0.125 nmol/g (10.6 mg/kg). In some embodiments, a dosage of about 0.1, 0.25, 0.5, 0.75, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 35, 40, 45, 50, or 55 mg/kg, or any range derivable therein, may be administered to a mammalian subject, such as a human. For example, a dosage of about 0.5-20 mg/kg, or any range derivable therein, may be administered to a human subject.

The therapeutic compound may be administered once or repeatedly to the same subject. In some embodiments, the therapeutic compound is administered repeatedly to the same subject (e.g., human patient), with at least 1, 2, 3, 4, 5, 6, 7, or more days, or 1, 2, 3, or 4 weeks separating the administrations. As shown in the below examples, improved efficacy was observed when therapeutic compound (FcF₂-MMAE) was injected every 7 rather than every 4 days. The therapeutic compound may be repeatedly administered to the same subject weekly or over a period of months or longer, or for as long as the subject has a disease (e.g., cancer). In some embodiments, the therapeutic compound is administered once every 4 days (q4d), once every seven days (q7d), once every 21 days (q21d), or once every 28 days (q28d).

In any case, the composition may comprise various antioxidants to retard oxidation of one or more component. Additionally, the prevention of the action of microorganisms can be brought about by preservatives such as various antibacterial and antifungal agents, including but not limited to parabens (e.g., methylparabens, propylparabens), chlorobutanol, phenol, sorbic acid, thimerosal or combinations thereof.

In embodiments where compositions are provided in a liquid form, a carrier can be a solvent or dispersion medium comprising but not limited to, water, ethanol, polyol (e.g., glycerol, propylene glycol, liquid polyethylene glycol, etc.), lipids (e.g., triglycerides, vegetable oils, liposomes) and combinations thereof. The proper fluidity can be maintained, for example, by the use of a coating, such as lecithin; by the maintenance of the required particle size by dispersion in carriers such as, for example liquid polyol or lipids; by the use of surfactants such as, for example hydroxypropylcellulose; or combinations thereof such methods. In many cases, it will be preferable to include isotonic agents, such as, for example, sugars, sodium chloride or combinations thereof.

Sterile injectable solutions are prepared by incorporating the active compounds in the required amount in the appropriate solvent with various of the other ingredients enumerated above, as required, followed by filtered sterilization. Generally, dispersions are prepared by incorporating the various sterilized active ingredients into a sterile vehicle which contains the basic dispersion medium and/or the other ingredients. In the case of sterile powders for the preparation of sterile injectable solutions, suspensions or emulsion, the preferred methods of preparation are vacuum-drying or freeze-drying techniques which yield a powder of the active ingredient plus any additional desired ingredient from a previously sterile-filtered liquid medium thereof. The liquid medium should be suitably buffered if necessary and the liquid diluent first rendered isotonic prior to injection with sufficient saline or glucose. The preparation of highly concentrated compositions for direct injection is also contemplated, where the use of DMSO as solvent is envisioned to result in extremely rapid penetration, delivering high concentrations of the active agents to a small area.

The composition must be stable under the conditions of manufacture and storage, and preserved against the contaminating action of microorganisms, such as bacteria and fungi. It will be appreciated that endotoxin contamination should be kept minimally at a safe level, for example, less than 0.5 ng/mg protein.

In particular embodiments, prolonged absorption of an injectable composition can be brought about by the use in the compositions of agents delaying absorption, such as, for example, aluminum monostearate, gelatin or combinations thereof.

### X. Cancers

A variety of cancer cells may be treated by methods and compositions provided herein. In some embodiments, the cancer cells express LGR4, LGR5 and/or LGR6. The cancer cells may be cancer stem cells (CSC), cancer cells with increased stemness, or a cancer that express LGR4, LGR5 or LGR6 independent of functional classification.

In some embodiments, the cancer includes CSC. Solid tumors have cell subpopulations that exhibit many of the characteristics of stem cells such as the ability to form spheroids or initiate new tumors. As for stem cells residing in organized epithelia, many CSC require an RSPO to grow vigorously when cultured (Sato et al., 2009; Barker et al., 2010). Elimination of the small fraction of cells in a tumor that are CSC may reduce or stop further tumor expansion by reducing the supply of the more differentiated cells that make up the bulk of the population.

In some embodiments, the cancer is an ovarian cancer. High grade serous ovarian cancer can arise from either the ovarian surface epithelium or the epithelium of the fallopian tube, although the latter predominates (Zhang et al., 2019). Lineage tracing studies in mice suggest that LGR5 marks a stem cell population in the ovary, and LGR6 marks stem cells in the mouse and human fallopian tube epithelium (de Lau et al., 2014; Kessler et al., 2015; Zhang et al., 2019). Data from the TCGA show that high grade serous ovarian cancer can express high levels of LGR5 and LGR6 mRNA. In addition, with the exception of mesothelioma, ovarian cancer has the highest median expression of RSPO1 mRNA when compared to all other tumor types in the database (Schindler et al., 2017). Without being bound by any theory, this data supports the idea that ovarian cancers may rely on RSPO1 to support their CSC population. As shown in the below examples, data is provided herein that supports the idea that compounds provided herein can utilize the receptor binding domain of RSPO1 armed with a cytotoxin to selectively bind to cancer cells (e.g., ovarian cancer cells that express LGR5/LGR6) and deliver the cytotoxin to the cancer cells.

Cancer cells that may be treated with cell targeting constructs according to the embodiments include but are not limited to cells from the bladder, blood, bone, bone marrow, brain, breast, colon, esophagus, gastrointestine, gum, head, kidney, liver, lung, nasopharynx, neck, ovary, prostate, skin, stomach, testis, tongue, or uterus. In addition, the cancer may specifically be of the following histological type, though it is not limited to these: neoplasm, malignant; carcinoma; carcinoma, undifferentiated; giant and spindle cell carcinoma; small cell carcinoma; papillary carcinoma; squamous cell carcinoma; lymphoepithelial carcinoma; basal cell carcinoma; pilomatrix carcinoma; transitional cell carcinoma; papillary transitional cell carcinoma; adenocarcinoma; gastrinoma, malignant; cholangiocarcinoma; hepatocellular carcinoma; hepatobiliary cancer, combined hepatocellular carcinoma and cholangiocarcinoma; trabecular adenocarcinoma; adenoid cystic carcinoma; adenocarcinoma in adenomatous polyp; adenocarcinoma, familial polyposis coli; solid carcinoma; carcinoid tumor, malignant; branchiolo-alveolar adenocarcinoma; papillary adenocarcinoma; chromophobe carcinoma; acidophil carcinoma; oxyphilic adenocarcinoma; basophil carcinoma; clear cell adenocarcinoma; granular cell carcinoma; follicular adenocarcinoma; papillary and follicular adenocarcinoma; nonencapsulating sclerosing carcinoma; adrenal cortical carcinoma; endometroid carcinoma; skin appendage carcinoma; apocrine adenocarcinoma; sebaceous adenocarcinoma; ceruminous adenocarcinoma; mucoepidermoid carcinoma; cystadenocarcinoma; papillary cystadenocarcinoma; papillary serous cystadenocarcinoma; mucinous cystadenocarcinoma; mucinous adenocarcinoma; signet ring cell carcinoma; infiltrating duct carcinoma; medullary carcinoma; lobular carcinoma; inflammatory carcinoma; paget's disease, mammary; acinar cell carcinoma; adenosquamous carcinoma; adenocarcinoma w/squamous metaplasia; thymoma, malignant; ovarian stromal tumor, malignant; thecoma, malignant; granulosa cell tumor, malignant; androblastoma, malignant; sertoli cell carcinoma; leydig cell tumor, malignant; lipid cell tumor, malignant; paraganglioma, malignant; extra-mammary paraganglioma, malignant; pheochromocytoma; glomangiosarcoma; malignant melanoma; amelanotic melanoma; superficial spreading melanoma; melanoma in giant pigmented nevus; epithelioid cell melanoma; blue nevus, malignant; sarcoma; fibrosarcoma; fibrous histiocytoma, malignant; myxosarcoma; liposarcoma; leiomyosarcoma; rhabdomyosarcoma; embryonal rhabdomyosarcoma; alveolar rhabdomyosarcoma; stromal sarcoma; mixed tumor, malignant; mullerian mixed tumor; nephroblastoma; hepatoblastoma; carcinosarcoma; mesenchymoma, malignant; brenner tumor, malignant; phyllodes tumor, malignant; synovial sarcoma; mesothelioma, malignant; dysgerminoma; embryonal carcinoma; teratoma, malignant; struma ovarii, malignant; choriocarcinoma; mesonephroma, malignant; hemangiosarcoma; hemangioendothelioma, malignant; kaposi's sarcoma; hemangiopericytoma, malignant; lymphangiosarcoma; osteosarcoma; juxtacortical osteosarcoma; chondrosarcoma; chondroblastoma, malignant; mesenchymal chondrosarcoma; giant cell tumor of bone; ewing's sarcoma; odontogenic tumor, malignant; ameloblastic odontosarcoma; ameloblastoma, malignant; ameloblastic fibrosarcoma; pinealoma, malignant; chordoma; glioma, malignant; ependymoma; astrocytoma; protoplasmic astrocytoma; fibrillary astrocytoma; astroblastoma; glioblastoma; oligodendroglioma; oligodendroblastoma; primitive neuroectodermal; cerebellar sarcoma; ganglioneuroblastoma; neuroblastoma; retinoblastoma; olfactory neurogenic tumor; meningioma, malignant; neurofibrosarcoma; neurilemmoma, malignant; granular cell tumor, malignant; malignant lymphoma; hodgkin's disease; hodgkin's; paragranuloma; malignant lymphoma, small lymphocytic; malignant lymphoma, large cell, diffuse; malignant lymphoma, follicular; mycosis fungoides; other specified non-hodgkin's lymphomas; malignant histiocytosis; multiple myeloma; mast cell sarcoma; immunoproliferative small intestinal disease; leukemia; lymphoid leukemia; plasma cell leukemia; erythroleukemia; lymphosarcoma cell leukemia; myeloid leukemia; basophilic leukemia; eosinophilic leukemia; monocytic leukemia; mast cell leukemia; megakaryoblastic leukemia; myeloid sarcoma; and hairy cell leukemia.

### VI. Combination Therapies

In order to increase the effectiveness of a therapeutic compound of the present disclosure, it may be desirable to combine these compositions with other agents effective in the treatment of the disease of interest. In some embodiments, the therapeutic compound (e.g., cell-targeted cytotoxic agent comprising an auristatin) is administered to a mammalian subject in combination with a second anti-cancer agent or therapy, to treat a cancer in the subject. In some embodiments, a targeted cytotoxic agent of the present disclosure may be administered to a subject in combination with an immunotherapeutic, an anti-bacterial agent (e.g., an antibiotic) or an antiviral agent, to treat a bacterial or viral infection in the subject, respectively.

As a non-limiting example, the treatment of cancer may be implemented with a therapeutic compound of the present disclosure along with other anti-cancer agents. An “anti-cancer” agent is capable of negatively affecting cancer in a subject, for example, by killing cancer cells, inducing apoptosis in cancer cells, reducing the growth rate of cancer cells, reducing the incidence or number of metastases, reducing tumor size, inhibiting tumor growth, reducing the blood supply to a tumor or cancer cells, promoting an immune response against cancer cells or a tumor, preventing or inhibiting the progression of cancer, or increasing the lifespan of a subject with cancer. More generally, these other compositions would be provided in a combined amount effective to kill or inhibit proliferation of the cell. This process may involve contacting the cells with agent(s) or multiple factor(s) at the same time. This may be achieved by contacting the cell with a single composition or pharmacological formulation that includes both agents, or by contacting the cell with two distinct compositions or formulations, at the same time, wherein one composition includes the anti-cancer peptide or nanoparticle complex and the other includes the second agent(s). In some embodiments, an anti-cancer peptide is one agent, and a cell-targeted cytotoxic agent (e.g., cell-targeted cytotoxic agent comprising an auristatin) is the other agent.

Treatment with the therapeutic compound of the present disclosure may precede or follow the other agent treatment by intervals ranging from minutes to weeks. In embodiments where the other agent and therapeutic compound are applied separately to the cell, one would generally ensure that a significant period of time did not expire between the time of each delivery, such that the agent and the therapeutic compound would still be able to exert an advantageously combined effect on the cell. In such instances, it is contemplated that one may contact the cell with both modalities within about 12-24 hours of each other and, more preferably, within about 6-12 hours of each other. In some situations, it may be desirable to extend the time period for treatment significantly where several days (e.g., 2, 3, 4, 5, 6 or 7 days) to several weeks (e.g., 1, 2, 3, 4, 5, 6, 7 or 8 weeks) lapse between the respective administrations. A wide range of dosing schedules may be employed.

Various combinations may be employed, where the therapeutic compound-based therapy is “A” (e.g., administration of a cell-targeted cytotoxic agent comprising an auristatin) and the secondary agent, such as radiotherapy, chemotherapy or anti-inflammatory agent, is “B”:

A/B/A B/A/B B/B/A A/A/B A/B/B B/A/A A/B/B/B B/A/B/B
B/B/B/A B/B/A/B A/A/B/B A/B/A/B A/B/B/A B/B/A/A
B/A/B/A B/A/A/B A/A/A/B B/A/A/A A/B/A/A A/A/B/A

In certain embodiments, administration of the therapy of the present embodiments to a patient will follow general protocols for the administration of chemotherapeutics, taking into account the toxicity, if any, of the vector. It is expected that the treatment cycles would be repeated as necessary. It also is contemplated that various standard therapies, as well as surgical intervention, may be applied in combination with the described hyperproliferative cell therapy.

### A. Chemotherapy

Cancer therapies also include a variety of combination therapies. In some aspects a therapeutic compound (e.g., cell-targeted cytotoxic agent comprising an auristatin) of the present disclosure is administered (or formulated) in conjunction with a chemotherapeutic agent. For example, in some aspects the chemotherapeutic agent is a protein kinase inhibitor such as a EGFR, VEGFR, AKT, Erb1, Erb2, ErbB, Syk, Bcr-Abl, JAK, Src, GSK-3, PI3K, Ras, Raf, MAPK, MAPKK, mTOR, c-Kit, eph receptor or BRAF inhibitors. Nonlimiting examples of protein kinase inhibitors include Afatinib, Axitinib, Bevacizumab, Bosutinib, Cetuximab, Crizotinib, Dasatinib, Erlotinib, Fostamatinib, Gefitinib, Imatinib, Lapatinib, Lenvatinib, Mubritinib, Nilotinib, Panitumumab, Pazopanib, Pegaptanib, Ranibizumab, Ruxolitinib, Saracatinib, Sorafenib, Sunitinib, Trastuzumab, Vandetanib, AP23451, Vemurafenib, MK-2206, GSK690693, A-443654, VQD-002, Miltefosine, Perifosine, CAL101, PX-866, LY294002, rapamycin, temsirolimus, everolimus, ridaforolimus, Alvocidib, Genistein, Selumetinib, AZD-6244, Vatalanib, P1446A-05, AG-024322, ZD1839, P276-00, GW572016 or a mixture thereof.

Yet further combination chemotherapies include, for example, alkylating agents such as thiotepa and cyclosphosphamide; alkyl sulfonates such as busulfan, improsulfan and piposulfan; aziridines such as benzodopa, carboquone, meturedopa, and uredopa; ethylenimines and methylamelamines including altretamine, triethylenemelamine, trietylenephosphoramide, triethiylenethiophosphoramide and trimethylolomelamine; acetogenins (especially bullatacin and bullatacinone); a camptothecin (including the synthetic analogue topotecan); bryostatin; callystatin; CC-1065 (including its adozelesin, carzelesin and bizelesin synthetic analogues); cryptophycins (particularly cryptophycin 1 and cryptophycin 8); dolastatin; duocarmycin (including the synthetic analogues, KW-2189 and CB1-TM1); eleutherobin; pancratistatin; a sarcodictyin; spongistatin; nitrogen mustards such as chlorambucil, chlornaphazine, cholophosphamide, estramustine, ifosfamide, mechlorethamine, mechlorethamine oxide hydrochloride, melphalan, novembichin, phenesterine, prednimustine, trofosfamide, uracil mustard; nitrosureas such as carmustine, chlorozotocin, fotemustine, lomustine, nimustine, and ranimnustine; antibiotics such as the enediyne antibiotics (e.g., calicheamicin, especially calicheamicin gammalI and calicheamicin omegaI1; dynemicin, including dynemicin A; bisphosphonates, such as clodronate; an esperamicin; as well as neocarzinostatin chromophore and related chromoprotein enediyne antiobiotic chromophores, aclacinomysins, actinomycin, authrarnycin, azaserine, bleomycins, cactinomycin, carabicin, carminomycin, carzinophilin, chromomycinis, dactinomycin, daunorubicin, detorubicin, 6-diazo-5-oxo-L-norleucine, doxorubicin (including morpholino-doxorubicin, cyanomorpholino-doxorubicin, 2-pyrrolino-doxorubicin and deoxydoxorubicin), epirubicin, esorubicin, idarubicin, marcellomycin, mitomycins such as mitomycin C, mycophenolic acid, nogalarnycin, olivomycins, peplomycin, potfiromycin, puromycin, quelamycin, rodorubicin, streptonigrin, streptozocin, tubercidin, ubenimex, zinostatin, zorubicin; anti-metabolites such as methotrexate and 5-fluorouracil (5-FU); folic acid analogues such as denopterin, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine; androgens such as calusterone, dromostanolone propionate, epitiostanol, mepitiostane, testolactone; anti-adrenals such as mitotane, trilostane; folic acid replenisher such as frolinic acid; aceglatone; aldophosphamide glycoside; aminolevulinic acid; eniluracil; amsacrine; bestrabucil; bisantrene; edatraxate; defofamine; demecolcine; diaziquone; elformithine; elliptinium acetate; an epothilone; etoglucid; gallium nitrate; hydroxyurea; lentinan; lonidainine; maytansinoids such as maytansine and ansamitocins; mitoguazone; mitoxantrone; mopidanmol; nitraerine; pentostatin; phenamet; pirarubicin; losoxantrone; podophyllinic acid; 2-ethylhydrazide; procarbazine; PSK polysaccharide complex; razoxane; rhizoxin; sizofiran; spirogermanium; tenuazonic acid; triaziquone; 2,2′,2″-trichlorotriethylamine; trichothecenes (especially T-2 toxin, verracurin A, roridin A and anguidine); urethan; vindesine; dacarbazine; mannomustine; mitobronitol; mitolactol; pipobroman; gacytosine; arabinoside (“Ara-C”); cyclophosphamide; taxoids, e.g., paclitaxel and docetaxel gemcitabine; 6-thioguanine; mercaptopurine; platinum coordination complexes such as cisplatin, oxaliplatin and carboplatin; vinblastine; platinum; etoposide (VP-16); ifosfamide; mitoxantrone; vincristine; vinorelbine; novantrone; teniposide; edatrexate; daunomycin; aminopterin; xeloda; ibandronate; irinotecan (e.g., CPT-11); topoisomerase inhibitor RFS 2000; difluorometlhylornithine (DMFO); retinoids such as retinoic acid; capecitabine; carboplatin, procarbazine, plicomycin, gemcitabien, navelbine, farnesyl-protein tansferase inhibitors, transplatinum, and pharmaceutically acceptable salts, acids or derivatives of any of the above. In certain embodiments, the compositions provided herein may be used in combination with gefitinib. In other embodiments, the present embodiments may be practiced in combination with Gleevac (e.g., from about 400 to about 800 mg/day of Gleevac may be administered to a patient). In certain embodiments, one or more chemotherapeutic may be used in combination with the compositions provided herein.

### B. Radiotherapy

Radiotherapy has been used extensively in treatments and includes what are commonly known as γ-rays, X-rays, and/or the directed delivery of radioisotopes to tumor cells. Other forms radiotherapy are also contemplated such as microwaves and UV-irradiation. These factors may affect a broad range of damage on DNA, on the precursors of DNA, on the replication and repair of DNA, and on the assembly and maintenance of chromosomes. Dosage ranges for X-rays range from daily doses of 50 to 200 roentgens for prolonged periods of time (3 to 4 weeks), to single doses of 2000 to 6000 roentgens. Dosage ranges for radioisotopes vary widely, and depend on the half-life of the isotope, the strength and type of radiation emitted, and the uptake by the neoplastic cells.

The terms “contacted” and “exposed,” when applied to a cell, are used herein to describe the process by which a therapeutic composition and a chemotherapeutic or radiotherapeutic agent are delivered to a target cell or are placed in direct juxtaposition with the target cell. To achieve cell killing or stasis, both agents are delivered to a cell in a combined amount effective to kill the cell or prevent it from dividing.

### C. Immunotherapy

Immunotherapeutics, generally, rely on the use of immune effector cells and molecules to target and destroy cancer cells. The immune effector may be, for example, an antibody specific for some marker on the surface of a tumor cell. For example, the immunotherapy may be an antibody such as, e.g., anti-PD-L1 antibody or an anti-CTLA4 antibody. In some embodiments, the immunotherapy is an antibody drug conjugate (ADC).

The antibody alone may serve as an effector of therapy or it may recruit other cells to actually affect cell killing. The antibody also may be conjugated to a drug or toxin (chemotherapeutic, radionuclide, ricin A chain, cholera toxin, pertussis toxin, etc.) and serve merely as a targeting agent. Alternatively, the effector may be a lymphocyte carrying a surface molecule that interacts, either directly or indirectly, with a tumor cell target. Various effector cells include cytotoxic T cells and NK cells.

Immunotherapy, thus, could be used as part of a combined therapy, in conjunction with a serine protease therapy of the present embodiments. The general approach for combined therapy is discussed below. Generally, the tumor cell must bear some marker that is amenable to targeting, i.e., is not present on the majority of other cells. Many tumor markers exist and any of these may be suitable for targeting in the context of the present embodiments. Common tumor markers include carcinoembryonic antigen, prostate specific antigen, urinary tumor associated antigen, fetal antigen, tyrosinase (p97), gp68, TAG-72, HMFG, Sialyl Lewis Antigen, MucA, MucB, PLAP, estrogen receptor, laminin receptor, erb B and p155.

### D. Gene Therapy

In yet another embodiment, the secondary treatment is a gene therapy in which a therapeutic polynucleotide is administered before, after, or at the same time as the therapeutic composition. Viral vectors for the expression of a gene product are well known in the art, and include such eukaryotic expression systems as adenoviruses, adeno-associated viruses, retroviruses, herpesviruses, lentiviruses, poxviruses including vaccinia viruses, and papiloma viruses, including SV40. Alternatively, the administration of expression constructs can be accomplished with lipid based vectors such as liposomes or DOTAP:cholesterol vesicles. All of these method are well known in the art (see, e.g. Sambrook et al., 1989; Ausubel et al., 1998; Ausubel, 1996).

Delivery of a vector encoding one of the following gene products will have a combined anti-hyperproliferative effect on target tissues. A variety of proteins are encompassed within the present embodiments and are well known in the art.

### E. Surgery

Approximately 60% of persons with cancer will undergo surgery of some type, which includes preventative, diagnostic or staging, curative and palliative surgery. Curative surgery is a cancer treatment that may be used in conjunction with other therapies, such as the treatments provided herein, chemotherapy, radiotherapy, hormonal therapy, gene therapy, immunotherapy and/or alternative therapies.

Curative surgery includes resection in which all or part of cancerous tissue is physically removed, excised, and/or destroyed. Tumor resection refers to physical removal of at least part of a tumor. In addition to tumor resection, treatment by surgery includes laser surgery, cryosurgery, electrosurgery, and microscopically controlled surgery (Mohs' surgery). It is further contemplated that the present embodiments may be used in conjunction with removal of superficial cancers, precancers, or incidental amounts of normal tissue.

Upon excision of part of all of cancerous cells, tissue, or tumor, a cavity may be formed in the body. Treatment may be accomplished by perfusion, direct injection or local application of the area with an additional anti-cancer therapy. Such treatment may be repeated, for example, every 1, 2, 3, 4, 5, 6, or 7 days, or every 1, 2, 3, 4, and 5 weeks or every 1, 2, 3, 4, 5, 6, 7, 8, 9, 10, 11, or 12 months. These treatments may be of varying dosages as well.

### IV. Examples

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1

### Compounds for Targeting LGR5-Expressing Stem Cells in Ovarian Cancer

### Design of FcF2-His

The upper portion of FIG. 1 presents a schematic diagram of FcF2-His. The protein contains a variant human IgG1 Fc domain (Lee et al., 2019) connected through two Gly₄Ser spacers to the Fu₁-Fu₂ domains of human RSPO1. Two further Gly₄Ser spacers separate the Fu₁-Fu₂ domains from the LPETGG sortase recognition sequence which is followed by an 8×His tag that facilitates purification. Each Fc-(Fu₁-Fu₂)-LPETGG-His (abbreviated FcF2-His) sequence contains 391 amino acids. The Fc domain causes dimerization via 3 intermolecular disulfide bonds, the bond that links the Fc to the light chain in an antibody and the two bonds that link the Fc domains together at their hinge region. The resulting protein has an overall calculated MW of 85,284 Da. Glycosylation is present on the Fc domain and on residue N137 in Fu₁-Fu2 domain (RSPO1 numbering).

### Sortase-Mediated Conjugation of Monomethyl Auristatin E (MMAE)

The lower portion of FIG. 1 shows a schematic of the conversion of FcF2-His to FcF2-MMAE. The sortase enzyme cleaves the LPETGG sequence between the threonine and glycine and forms a transient thioester bond with a cysteine in the active site of the enzyme which is subsequently attacked by the N-terminal glycine of the protease sensitive GGG-vc-PAB-MMAE linker. The His tag is lost in the reaction. This results in the precise covalent loading of one molecule of MMAE on each arm of the FcF2-His and the production of a homogeneous population of conjugated FcF2-MMAE molecules.

### FcF2-MMAE Production, Purification and Characterization

FcF2-His was produced by transient transfection of the vector pcDNA3.1-FcF2-8×His into HEK293E cells and purified from cell supernates by capture on Ni-NTA resin and subsequent ion exchange chromatography. The FcF2-His was then reacted with sortase-His and GGG-vc-PAB-MMAE for 4 h at 37° C. to produce FcF2-MMAE. Across 14 batches the average yield of FcF2-His was 23.4±2.3 mg/L; sortase efficiency was 77±8.6%; and, the yield of final product was 18.8±3.5 mg/L (mean±SEM).

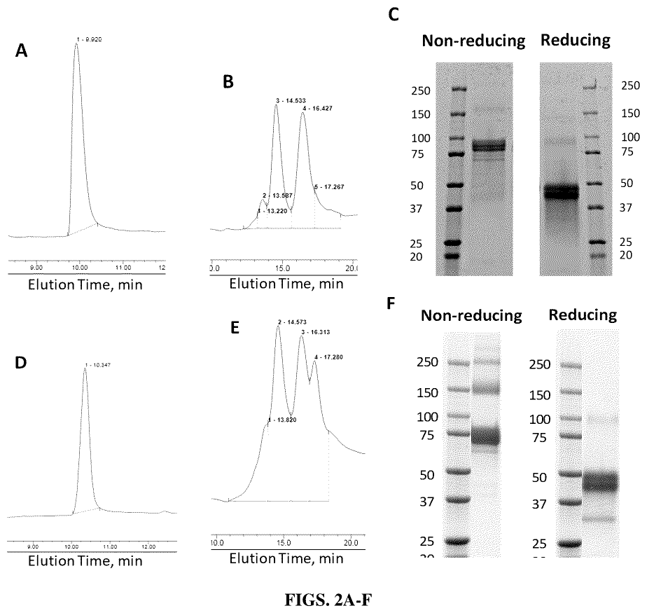

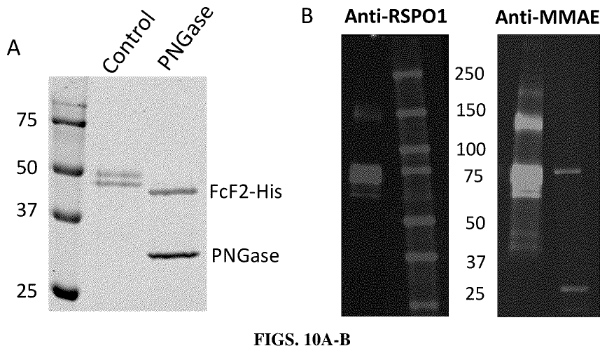

FIG. 2 shows the results of characterization of FcF2-His and FcF2-MMAE by reverse-phase HPLC, size exclusion chromatography and reducing and non-reducing SDS-PAGE. On reverse-phase HPLC analysis using a C4 column both forms of the molecule run as a single well-defined peak (FIGS. 2 A & D). Analysis on a HPLC 300 size-exclusion column indicated that FcF2-His and FcF2-MMAE exist in solution both as a dimer, and a dimer of the already dimeric molecule (FIGS. 2 B & E), hereafter referred to as the tetramer. A small amount of higher MW species (7%) eluted just before the major tetramer peak of FcF2-His; FcF2-MMAE contains a third lower MW peak. The non-reducing SDS-PAGE analysis of FcF2-His (FIG. 2C) showed that, in the presence of the SDS detergent in the loading buffer, most of the tetramer runs at a MW consistent with the size of the FcF2-His dimer (85.3 kD). Under reducing conditions, the bulk of the protein runs as a doublet at ˜42-46 kD. To refine understanding of this doublet, the FcF2-His was treated with PNGase to remove the N-linked sugars. As shown in FIG. 10A, this resulted in the collapse of the doublet to a single band consistent with the conclusion that the doublet is attributable to differential glycosylation. When the single glycosylation site in the Fu2 domain at N137 was mutated to an alanine, the doublet was lost. Extensive comparison of the wild type and N137A forms of R1FF-MMAE indicated no change in cytotoxicity or plasma pharmacokinetics indicating that glycosylation at this site had little functional consequence with respect to these parameters.

Non-reducing SDS-PAGE analysis of FcF2-MMAE (FIG. 2 E) showed that, in the presence of the SDS detergent in the loading buffer, the bulk of the FcF2-MMAE runs at a MW of 72-78 kD and that, when run under reducing conditions, it runs at 42-48 kD in a manner similar to the behavior of FcF2-His under non-reducing and reducing conditions. Western blot analysis of FcF2-MMAE using anti-RSPO1 and anti-MMAE antibodies indicated that the major bands that stain for protein on the SDS-PAGE gels contain all components of the molecule (Fc, Fu₁-Fu₂, and MMAE) (FIG. 10B) and this was confirmed also for all the major peaks visible in the C4 and SEC300 profiles.

### In Vitro Cytotoxicity and Selectivity of FcF2-MMAE

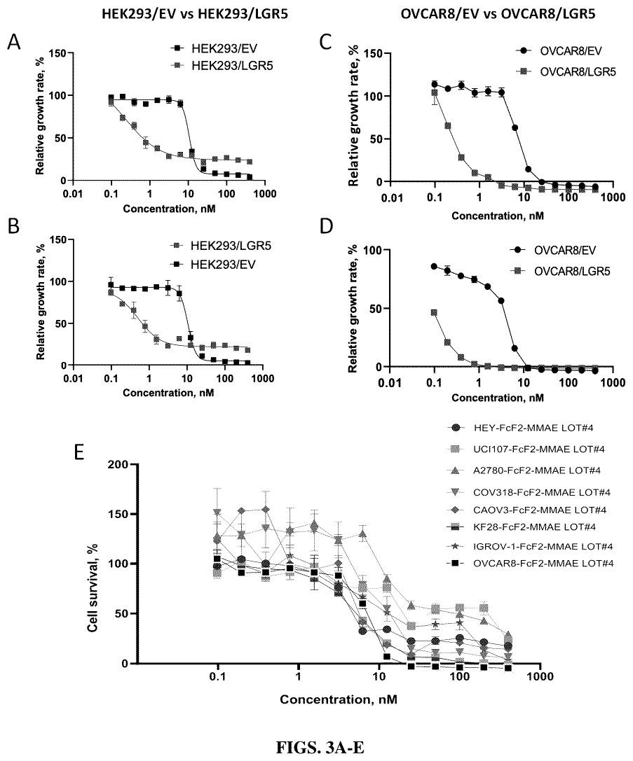

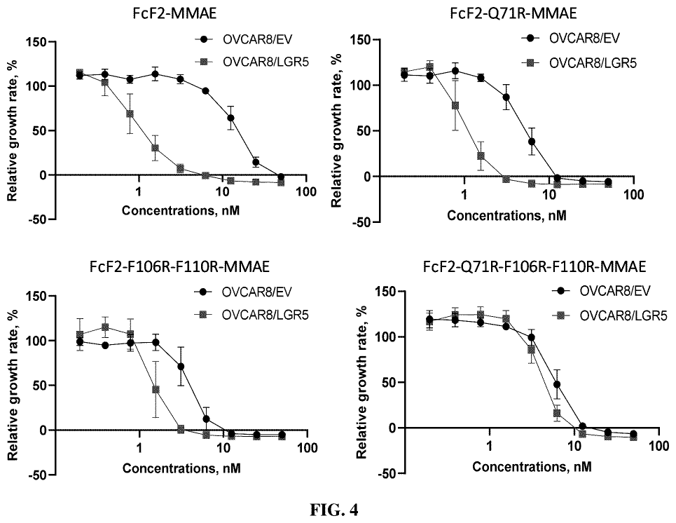

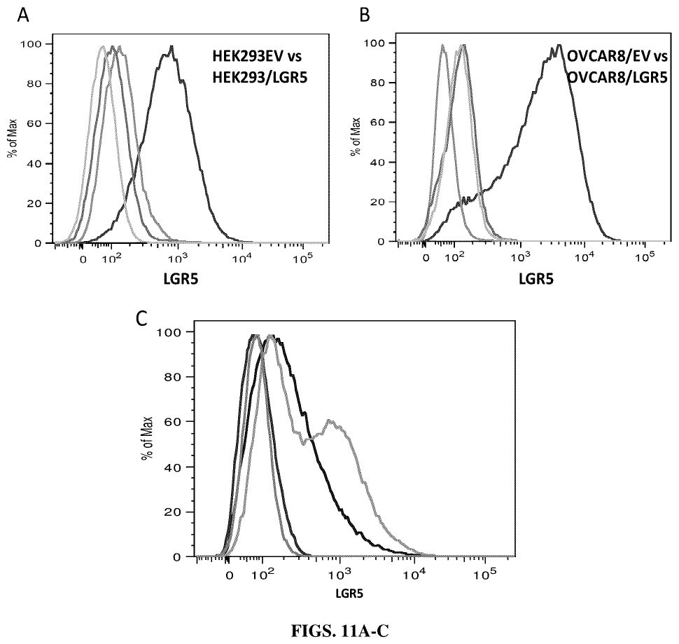

HEK293 cells and the human ovarian carcinoma cell line OVCAR8 were molecularly engineered to stably express increased levels of LGR5. The parental forms of both of these lines express variable but poorly defined levels of all 3 LGR receptors. Flow cytometric analysis using an antibody to LGR5 documented 8.7-fold higher levels in the HEK293/LGR5 cells than in the HEK293/EV controls; in the case of the OVCAR8 cells the difference was ˜25-fold (FIG. 11A and FIG. 11B, respectively). Cytotoxicity assays were carried out using a 120 h exposure to drug and the CCK8 reagent to assess viability. FIG. 3A&B shows LGR5 receptor-dependent greater cytotoxicity in the isogenic HEK293 pair; for two batches tested the HEK293/LGR5 cells were 21 and 46-fold more sensitive than the HEK293/EV cells. A large differential effect was also observed when tested using the OVCAR8/EV and OVCAR8/LGR5 isogenic pair; two recent batches demonstrated a 77- and 87-fold differential inhibition of cell growth (FIGS. 3C-D). Across recent batches that met all release criteria the mean IC₅₀ for the OVCAR8/EV cells was 4.5±1.1 nM (SEM) and for the OVACAR8/LGR5 cells it was 0.059±0.015 nM; the mean ratio of IC₅₀ values was 76±5 (N=3).

The question of whether the differential inhibition of growth of LGR5-poor and LGR5-rich isogenic pairs could be due to differences in sensitivity to free MMAE instead of Fu₁-Fu₂-directed targeting was addressed by using the same assay to determine IC₅₀ values for free MMAE. HEK293/LGR5 cells were 1.6-fold more resistant to free MMAE than the HEK293/EV cells (19.2±0.4 vs 30.5±2.5, p=0.03 N=3). The OVCAR8/LGR5 cells were 1.5-fold more resistant to free MMAE than the OVCAR8/EV cells (IC₅₀ values 129 vs 85 nM, respectively). Thus, both of the LG5R-rich cell types were actually slightly resistant to free MMAE providing confidence that the selectivity exhibited by FcF2-MMAE is due to targeting by the Fu₁-Fu₂ domains.

Further evidence was provided by the observation that treatment of a population of OVCAR8 cells with FcF2-MMAE deleted it of LGR-positive cells. A mixed population of OVCAR8 cells expressing low and higher levels of LGR5 cells were exposed to 10 nM FcF2-MMAE for 96 h. Prior to treatment flow cytometric analysis demonstrated a bi-modal distribution of LGR5-expressing cells (FIG. 11C). FcF2-MMAE treatment caused the loss of a large fraction of the cells expressing high levels of LGR5 but produced substantially less effect on the fraction of cells with low level LGR5 expression. In addition to differences in IC₅₀ values, this data provides evidence of differential killing as a function of LGR5 expression level.

### Cytotoxicity to Human Wild Type Ovarian Cancer Cell Lines

To assist in the selection of appropriate xenograft models for efficacy testing, the cytotoxicity of FcF2-MMAE was determined for a panel of 8 human ovarian carcinoma cell lines. The concentration-survival curves shown in FIG. 3E indicate that the IC₅₀ values ranged from 3.8 to 29.6 nM; the IC₅₀ was <10 nM in 7 of 8 cell lines. Thus, although the level of the sum of all LGR expression is not known for these cell lines, the data indicates that FcF2-MMAE is very potent across this panel of ovarian carcinoma cell lines.

### Determinants of Cytotoxic Selectivity

RSPO1 is a bispecific ligand. The Fu₁ domain of RPSO1 binds to the ubiquitin ligase receptors ZNRF3 and RNF43 and the Fu2 domain binds to the LGR4, LGR5 or LGR6. The relative contribution of each of these types of receptors to the selectivity of FcF2-MMAE for the OVCAR8/EV and OVCAR8/LGR5 cells was explored by introducing mutations in one or the other Fu domain that were previously documented to disable binding to its cognate receptor (Peng et al., 2013; Xie et al., 2013; Zebisch et al., 2013; Zhang et al., 2020; Cui et al., 2021). The cytotoxicity of the mutant forms was tested in the in vitro OVCAR8/EV and OVCAR8/LGR5 model. The growth inhibition curves presented in FIG. 4 indicate that both domains contribute importantly to the selectivity of FcF2-MMAE. The Q71R mutation in Fu₁ reduced the IC₅₀ ratio from 19.5 to 5.1 (p=0.05), the F106R-F1110R mutations in Fu2 reduced the ratio to 2.1 (p=0.03), and when both sets of mutations were present selectivity was abolished. Thus, in this model system, the ability to bind to both types of receptors is of substantial importance to the successful internalization of FcF2-MMAE and the liberation of free MMAE.

### Pharmacokinetics of FcF2-MMAE in Mice

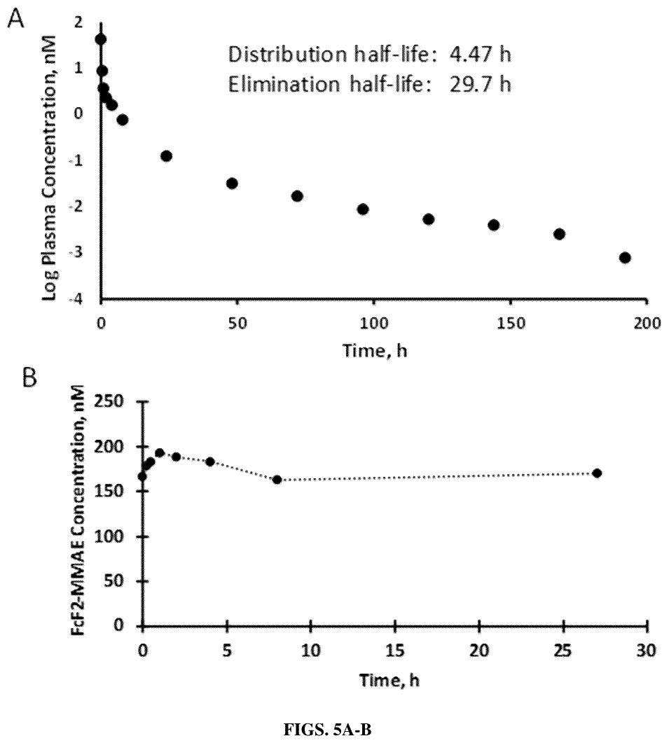

BALB/c mice were given a bolus injection of 0.1 nmol/g (9 ug/g) FcF2-MMAE and plasma samples were obtained from 3 mice at each sampling time point. The concentration of FcF2-MMAE was measured using an ELISA with monoclonal capture and polyclonal detection antibodies of different species prepared by immunization with RSPO1 (LLQ 1.5 pmol/ml). FIG. 5A presents the composite plasma decay curve; analysis with WinNonLin curve-fitting software yielded estimates of 4.47 h for the distribution half-life and 29.7 h for the terminal half-life. The initial half-life was shorter than anticipated. One mechanism by which FcF2-MMAE may be removed from the plasma compartment is through binding to red blood cells, white blood cells or platelets. However, when plasma spiked with FcF2-MMAE was added to sedimented formed elements to reconstitute their normal respective volumes, there was no significant removal of drug from the plasma fraction at 4° C. over 27 h (FIG. 5B). This suggests that the rapid initial half-life is largely due to distribution into tissues.

### Efficacy of FcF2-MMAE in Human Ovarian Cancer Xenograft Models

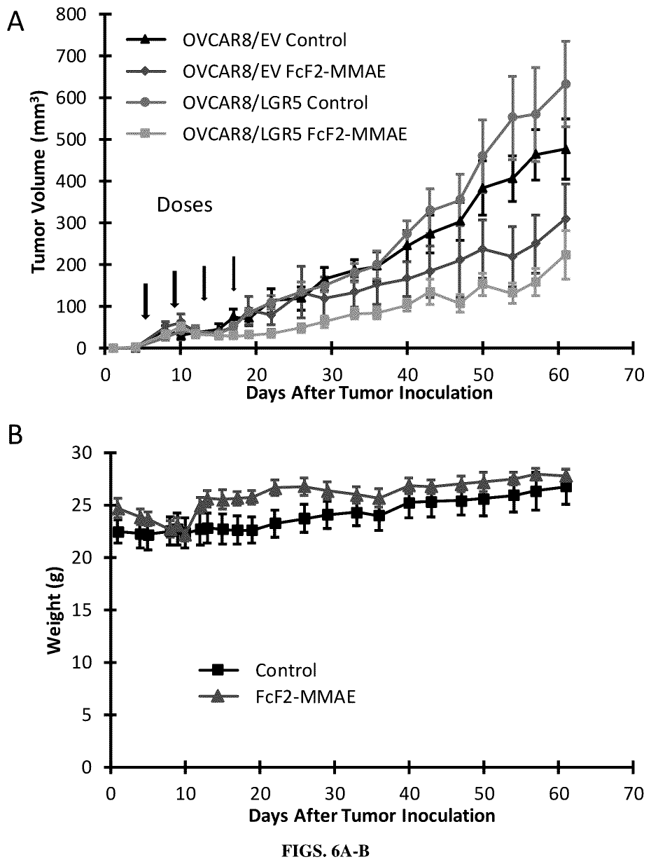

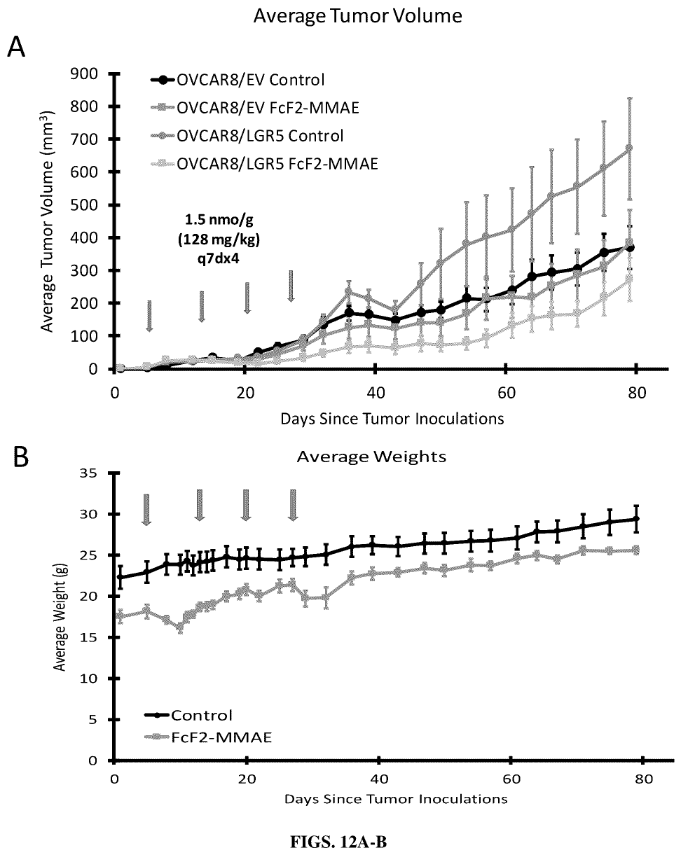

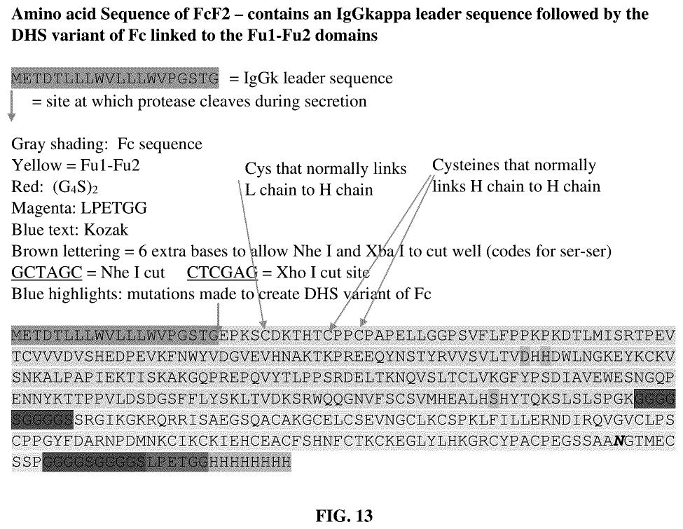

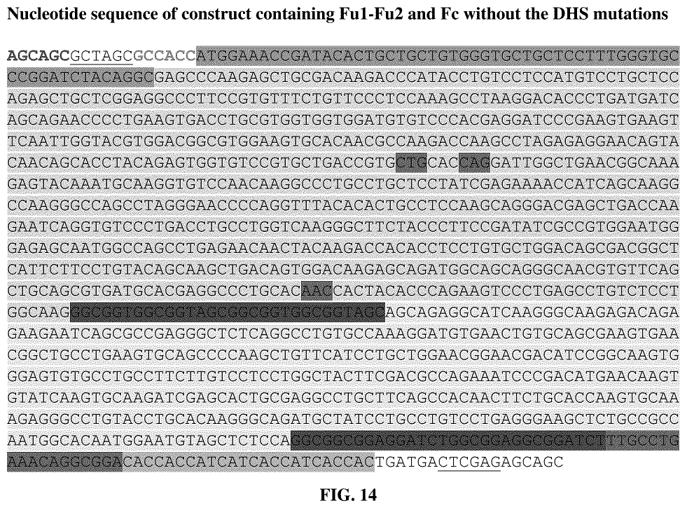

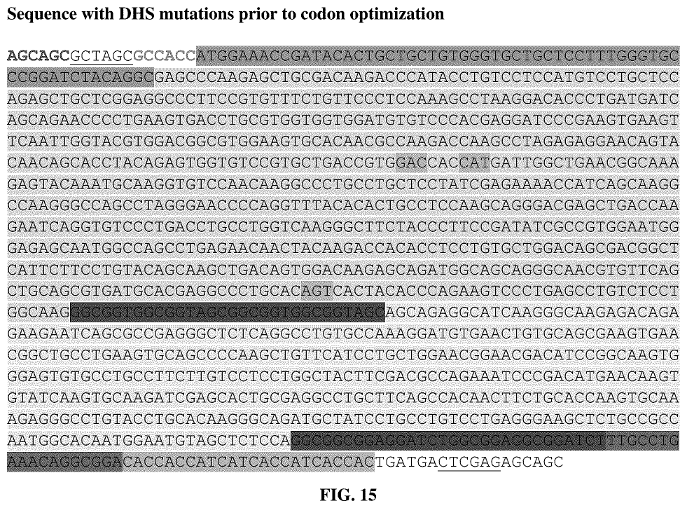

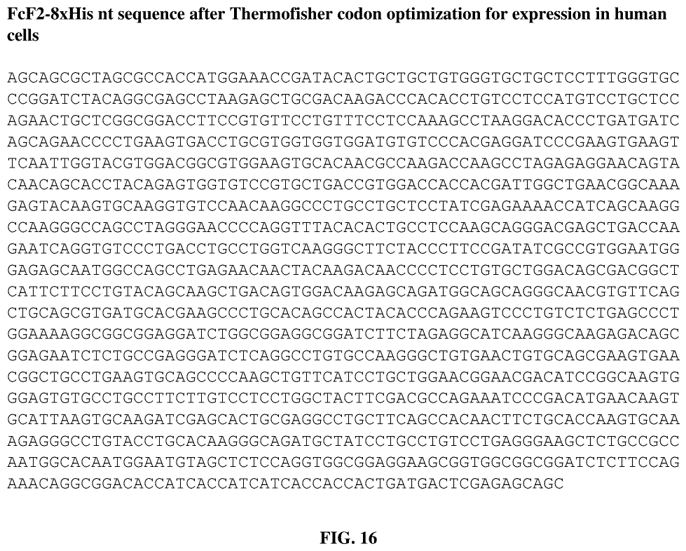

Xenografts established from the isogenic pair of OVCAR8/EV and OCVAR8/LGR5 cells were used to explore both the in vivo efficacy and selectivity of FcF2-MMAE. In the experiment presented in FIG. 6A, mice were treated with 0.5 nmol/g (42 mg/kg) every 4 days for 4 doses given by the IP route once tumors became palpable. FcF2-MMAE had much larger effect on the OVCAR8/LGR5 than on the OVCAR8/EV tumors. A difference in the growth rate was apparent by Day 20 and persisted to Day 60 at which point the FcF2-MMAE-treated OVCAR8/LGR5 tumors averaged only 35% of the size of the vehicle treated tumors. FcF2-MMAE was also effective at slowing the growth rate of the OVCAR8/EV cells consistent with evidence that they express some combination of the LGR receptors, but the curves for the drug and vehicle-treated tumors did not separate until after 30 days and, at 60 days, the FcF2-treated OVCAR8/EVs tumors averaged 63% as large as the control tumors. It is noteworthy that, for both OVCAR8/EV and OCVAR8/LGR5 tumors, the reduction in tumor growth rate was maintained for >1.5 months after the last dose of FcF2-MMAE, an effect consistent with targeting of stem cells in the tumor from which it is difficult to recover growth rate. The lack of dose-limiting toxicity prompted a second efficacy study of the same design but using a dose of 1.5 nmol/g and a q7dx4 schedule demonstrated the same large difference in the efficacy of FcF2-MMAE between the two types of xenografts (FIG. 12A).

FIG. 6B shows the change in average mouse weight during and after the course of 4 FcF2-MMAE injections at a dose of 0.5 nmol/g and FIG. 12B shows the same data for a dose of 1.5 nmol/g given on a q7dx4 schedule. The first injection caused a mean transient loss of body weight but it rapidly recovered and overall mice gained weight over the treatment period in both studies. There were no deaths in the animals treated with 0.5 nmol/g prior to sacrifice due to tumor burden in either the control or treatment groups. No observable adverse events including diarrhea, change in activity level, posture or grooming or reduction in food consumption occurred after the first dose of FcF2-MMAE.

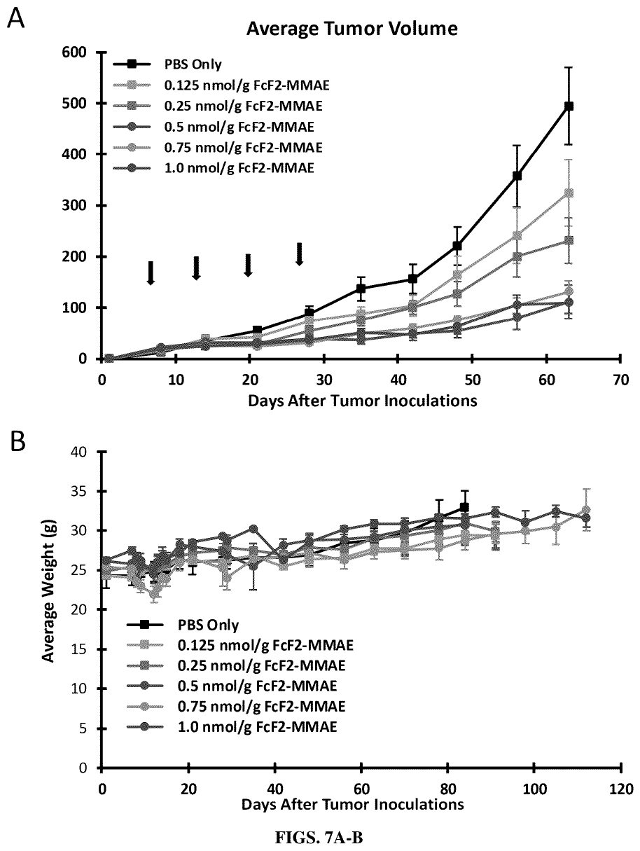

The OVCAR8/LGR5 model was used to explore the efficacy and toxicity of FcF2-MMAE as a function of dose using the more clinical-relevant every 7 day schedule. The growth curves presented in FIG. 7A document an increase in efficacy as the dose was increased from 0.125 to 1.0 nmol/g. Even a dose of 0.125 nmol/g (10.6 mg/kg) produced a significant decrease in growth rate (final tumor volume 66% of untreated control). As shown in FIG. 7B this dose produced no weight loss. Efficacy and maximal weight loss increased with dose up to 0.75 nmol/g; no further increase in either parameter was observed at 1.0 nmol/g. This experiment suggests a therapeutic window over an 8-fold dose range but FcF2-MMAE produced under GMP conditions will be required to refine this estimate.

### Efficacy of FcF2-MMAE in Wild Type Human Ovarian Cancer Xenograft Models

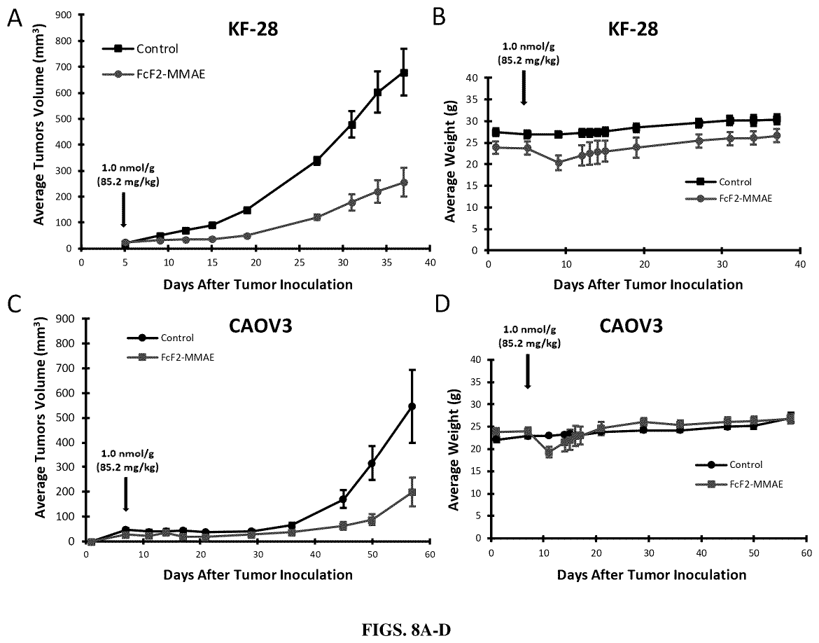

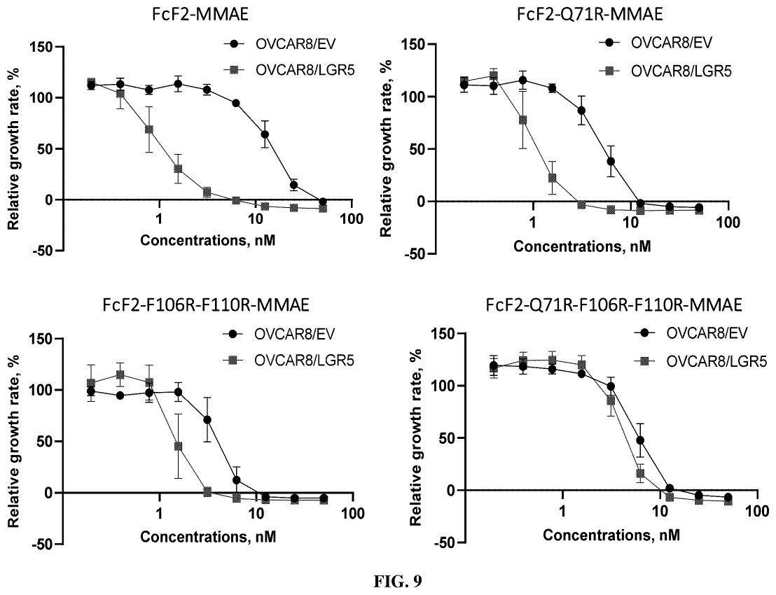

The efficacy of FcF2-MMAE was explored in a total of 3 ovarian cancer xenograft models established from cell lines that had not undergone any genetic modification to increase LGR5. Single doses of 1.0 nmol/g given IP at the time tumors became palpable produced long delays in tumor growth in the KF-28 and CAOV3 models in the absence of dose-limiting weight loss (FIG. 8 A/C). At a dose of 0.5 nmol/g FcF2-MMAE was active against IGROV8 and CAOV3 xenografts in the absence of significant toxicity in the form of weight loss on a q7dx4 schedule (FIG. 8 B/D). These data provide evidence for efficacy in 3 different human ovarian cancer models in the absence of molecular engineering to increase LGR5 expression. It is not currently possible to accurately define the level of expression of LGR4, LGR5 and LGR6 in these models so sensitivity cannot be linked to the expression of any one of these. However, the data is consistent with the concept that stem-like cells in each of these tumors express, in sum, enough of these receptors to be responsive to FcF2-MMAE.

Selectively targeting stem cells in tumors may reduce or limit tumor expansion and metastatic capacity of the cancer. The rationale for using the receptor-binding Fu₁-Fu₂ domain of RSPO1 to achieve this goal is based on its high affinity for LGR5 and LGR6 (˜3 nM) (Carmon et al., 2011), and evidence that expression of these receptors mark stem cells in tumors as they do in normal epithelia. This approach is of particular interest in the case of ovarian cancer because LGR5 and LGR6 mark stem cells in the ovarian surface and fallopian epithelia from which ovarian cancers arise (Zhang et al., 2019) and after transformation these tumors exhibit unusually high levels of expression of LGR5 and LGR6 (Schindler et al., 2017; Lee et al., 2020). The fact that the Fu₁-Fu₂ domain binds in a bispecific manner to both an LGR and either ZNRF3 or RNF43 favors specificity and, since it is part of a normal human protein, there is a reduced risk of immunogenicity.

FcF2-MMAE is different from the R1FF-MMAE molecule, which consisted of just the Fu₁-Fu₂ domain of RSPO1 linked to MMAE through a cleavable linker (Yu et al., 2021). Although R1FF-MMAE exhibited LGR5-dependent cytotoxicity and in vivo activity it did not have optimal pharmaceutical properties. The modifications made to create FcF2-MMAE were directed at: a) increasing the yield of protein from transiently transfected cultures by taking advantage of the chaperone function of Fc to improve folding; b) dimerizing it so that it carried two molecules of MMAE rather than one; c) increasing plasma half-life by including a mutant form of Fc with improved FcRn binding characteristics (Lee et al., 2019); and, d) increasing avidity of binding to the LGRs and ZNRF3/RNF43 by including 2 rather than just one copy of the Fu₁-Fu₂ domain. Achievement of these goals was evidenced by a markedly greater yield of the FcF2-His precursor, greater potency and selectivity when tested in the isogenic OVCAR8/EV and OVCAR8/LGR5 cells, a 6-fold increase in the terminal plasma half-life and improved efficacy in xenograft models. Importantly, dimerization of the two Fu₁-Fu₂ domains caused by the presence of the Fc domain did not compromise the efficiency of the sortase reaction which remained high.

FcF2-MMAE produces LGR5-dependent killing in vitro and differentially depletes the cells with the highest levels of this receptor. The mean difference of 76-fold in IC₅₀ values was sufficient to yield substantially greater in vivo efficacy against the LGR5-rich OVCAR8/LGR5 cells. The results of the cytotoxicity assays indicate that engagement with both the LGR and ubiquitin ligase receptors is important for optimum selectivity. Given its terminal half-life of 27.4 h, it was of interest that efficacy was better when injected every 7 rather than every 4 days. Consistent with its sub-nanomolar potency in vitro, in vivo activity was detected at a dose of just 0.125 nmol/g (10.6 mg/kg). Efficacy increased with dose up to a level approaching a maximum tolerated dose, yielding an estimate of an 8-fold therapeutic window which is substantially higher than that of many of the chemotherapeutic agents used for the treatment of ovarian cancer. Most importantly, FcF2-MMAE demonstrated cytotoxicity at <10 nM in all but one member of a panel of ovarian cell lines and had in vivo activity in 2 additional ovarian xenograft models established from cells expressing only endogenous un-manipulated levels of the LGRs. Although the sum total of expression of each individual member of the LGR4-6 family cannot be accurately determined due to differing affinities of available antibodies, and of each LGR for the RSPO1 Fu₁-Fu₂ domain in its dimeric form, this provides substantial assurance that they are high enough to allow FcF2-MMAE to be effective. LGR5 and LGR6 are expressed in the stem cells of many other types of cancer, supporting the use of FcF2-MMAE in treating a broad variety of cancers that express LGR5 and/or LGR6.

Cancers, including ovarian and other cancers, can be tested for LGR5/LGR6 expression. Expression may be higher in some cancers (e.g., malignant cells that have full stem cell capabilities). Cancers that have diminished stem call capacity but continue to express enough LGR5 or LGR6 may be sensitive to and benefit from treatment with FcF2-MMAE. Not all cells that express LGR5 can function as stem cells (Azkanaz et al., 2022). LGR5 expression is not confined to epithelial cells; LGR5 is expressed in some mesenchymal cells, but it is presently unclear if these cells have stem cell features (Kim et al., 2022). These results support the use of FcF2-MMAE for destruction of stem cells in tumors, and a similar selective killing is expected to be observed in malignant cells.

Ablation of LGR5-expressing cells in the mouse intestine does not destroy its epithelial integrity and recent studies suggest that this is due to the plasticity of transiently amplifying cells to regenerate cells with full stem cell capabilities (Tian et al., 2011; Azkanaz et al., 2022). However, the extent to which ovarian or other types of cancer retain such plasticity remains to be defined. Studies performed using organoid-derived colon cancer xenografts indicate that ablation of LGR5 can produce a relatively long-lasting response, and ADCs targeted to LGR5 produce good responses in colon cancer xenograft models (Junttila et al., 2015; Gong et al., 2016). The long duration of growth inhibition produced in the KF-28 and CAOV3 models was observed using a single dose of FcF2-MMAE.

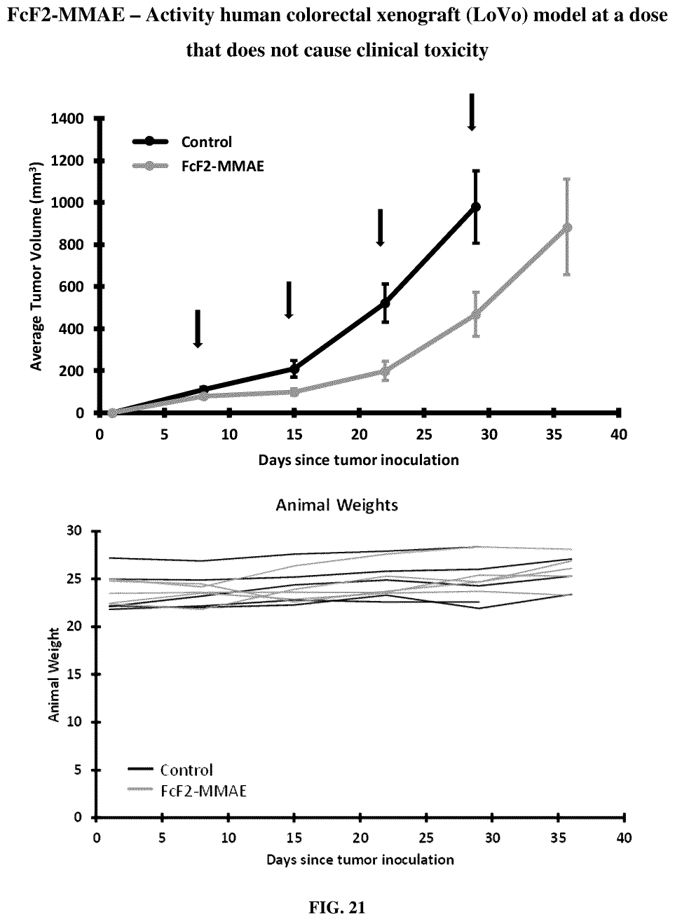

FcF2-MMAE activity was also tested in vivo using a xenograft mouse model of human colorectal cancer. Mice bearing human colorectal xenograft (LoVo) were administered FcF2-MMAE at a dose of 1 nmol/g every 7 days, for a total of 4 doses per mouse, and clinical toxicity was not observed at this dosage. FcF2-MMAE nonetheless resulted in a decrease in the average weight of the colorectal tumors, without altering the body weight of the mice. Results are shown in FIG. 21.

FcF2-MMAE produced unexpectedly little toxicity in mice at doses that resulted in anti-tumor activity. The differential effect on tumors versus normal tissues may be due to multiple factors in addition to a possible difference in the expression of LGR5 and LGR6 by tumor stem cells versus normal stem cells. Without wishing to be bound by any theory, normal epithelia may be more tolerant to loss of LGR5 positive cells as shown for the intestine (Tian et al., 2011; Junttila et al., 2015; Gong et al., 2016). In normal tissues, stem cells reside in highly structured protected niches surrounded by cells that provide support in the form of WNTs, RSPO1 and cytokines. FcF2-MMAE may have much better access to tumor stem cells than stem cells in a normal epithelial niche due to differences in microanatomy and physiology. To the extent that LGRs and ZNRF3/RNF43 are expressed on the luminal rather than basal surface of stem cells when they are in a normal polarized niche, the ability of RSPO1 in plasma to access the stem cell may be limited (de Vreede et al., 2022). However, polarization is lost after transformation and FcF2-MMAE may have better access to diffusely distributed receptors on cancer stem cells once the drug arrives in the stem cell environment. FcF2-MMAE contains only the Fu1-Fu2 domain of RSPO1 and is missing the long C-terminal TSP-BR domain that has been shown to mediate binding to proteoglycans that favors accumulation in the niches of normal tissues (Lebensohn and Rohatgi, 2018). The missing TSP-BR domain has also been reported to limit the ability of the remaining Fu₁-Fu₂ part of the molecule to activate WNT signaling via a non-LGR-dependent pathway, a potential cause of toxicity, although it does not impair receptor binding (Dubey et al., 2020).

FcF2-MMAE produced very few clinically observable adverse events in mice at therapeutically effective doses. It is presently unclear if the Fu₁-Fu₂ domain might drive some degree of unwanted proliferation of both normal and tumor tissues. Systemic administration of a large dose of full length RSPO1 produced rapid but transient up-regulation of WNT signaling in the small intestine, as detected by increases in AXIN2 and Ki-67 expression. A response was detectable at 3 h, peaked at 24 h, and largely resolved by 48 h (Kim et al., 2005). These results were not observed in association with any adverse clinical consequences. Repeated large daily doses of endogenous RSPO1 can produce a proliferative response in the stem cells of the jejunum (Zhou et al., 2013; Sun et al., 2021), liver (Sun et al., 2021) and skin (Weber et al., 2020) but this too is well-tolerated. RSPO1 can facilitate recovery from both radiation- and chemically-induced enteritis (Zhao et al., 2007; Zhao et al., 2009; Zhou et al., 2013). The results support the idea that even if FcF2-MMAE produces an increase in WNT signaling in vivo, the response would likely only be transient and not sustained when the drug is administered on a weekly schedule.

Historically it has been very difficult to develop high affinity antibodies selective for LGR5 and LGR6. Nevertheless several investigators have explored the use of antibody-drug conjugates (ADCs) that target LGR5 to deplete stem cells in gastrointestinal tumors (Junttila et al., 2015; Gong et al., 2016; Azhdarinia et al., 2018). While good responses were observed, the magnitude of LGR5-mediated selectivity was modest.

The data supports the idea that RSPO targeting may have several important advantages over ADCs. ADC development has not progressed and no anti-LGR5 ADCs have entered clinical trials. Multiple advantages of RSPO targeting may exist, including the following. First, FcF2-MMAE uses the natural ligand which binds with nanomolar affinity and may be rapidly internalized by endocytosis so that its cargo is delivered intracellularly. Second, Fu₁-Fu₂ armed with a cytotoxin has the potential to target all 3 of the LGR family members (LGR4, LGR5 and LGR6) and the two ubiquitin ligase receptors ZNRF3 and RNF43 at the same time whereas an ADC can target only a single LGR at a time. Thus, an Fu₁-Fu₂ domain armed with a cytotoxin has the potential of killing cells that have a low expression of the one type of the LGR or ubiquitin ligase receptors but substantial expression of another. Third, the precision with which the sortase reaction conjugates MMAE results in a more homogeneous population of molecules. Fourth, to the extent that ZNRF3/RNF43 are also expressed on CSC, the bispecific binding of the Fu₁-Fu₂ domain favors selectivity and an enhanced rate and extent of internalization. Fifth, there are tumors that over-express ZNRF3 and RNF43 independently of the LGRs. Since the Fu₁ domain binds to these two receptors it can target this type of tumor as well. Sixth, the Fu1-Fu2 domain may simultaneously engages both an LGR and ZNRF3 or RNF43. In essence this is the equivalent of a bispecific ADC; this type of ADC is currently of great interest because of their enhanced avidity, specificity and ability to cluster receptors and mediate enhanced internalization (Shim, 2020). These results support both Fu₁-Fu₂ as a targeting ligand to deliver a cytotoxic payload to cancerous cells that express LGR5/LGR6, and also the use of FcF2-MMAE to treat cancers that may include CSC.

### Example 2

### Materials and Methods

The following materials and methods were used in the experiments provided in Example 1.

Reagents and cell lines: Antibodies were from the following sources: anti-RSPO1, clone OTI11A9, OriGene, Inc; anti-MMAE, clone B11F11, Levena Biopharma; nti-hLGR5/GPR49 antibody Cat #MAB8078, R&D Systems; PE-conjugated anti-mouse IgG antibody Cat #F0102B, R&D Systems. Ni-NTA resin was purchased from Qiagen, and SP-sepharose and DEAE resins from GE healthcare Life Sciences. Propidium iodide was purchased from ThermoFisher (Cat #P3566). All ovarian cancer cell lines were acquired either from ATCC or from laboratories in the United States; all cell lines were STR verified at ATCC. The sortase plasmid vector pet30b-7M SrtA was purchased from Addgene. Sortase-6×His containing mutations P94R, E105K, E108Q, D160N, D165A, K190E, and K196T was produced in E. coli strain Rosetta and purified using Ni-NTA resin chromatography as previously described Yu et al. 2021. (Gly)₃-vc-PAB-MMAE was synthesized by Levena Biopharma. Plasma levels of FcF2-MMAE were determined using an ELISA kit from R&D Systems (DY4645-05)).

Synthesis and purification of FcF2-His: FcF2-His was produced by transient transfection of a pcDNA3.1 vector containing an insert coding for the Fu1-Fu2 domain of RSPO1 into HEK293E cells. Cells were grown in 300 ml of HEK293E Culture Media, which consists of 150 mL Gibco FreeStyle 293 Medium (Cat #12338-026, Thermofisher), 150 mL HyClone SFM4HEK293 media (Cat #82003-356), 6 mL Fetal Bovine Serum (Cat #26140-079, ThermoFisher), 333 uL G418 Sulfate (Cat #G8168, Sigma), and 333 uL Anti-clumping agent (Cat #0010057AE, ThermoFisher) in 1 L flasks on a platform rotating at 130 rpm. Cell supernates were harvested after 5 days of culture, centrifuged to sediment debris and then loaded onto a column containing Ni-NTA resin (Cat #30250, Qiagen) that was equilibrated with buffer containing 150 mM NaCl, 20 mM Tris, pH 7.6. After washing, the FcF2-His protein was eluted with buffer containing 300 mM imidazole, 150 mM NaCl, 20 mM Tris, pH 7.6. The eluates were diluted 1:3 with 20 mM Tris, pH 7.6 and then loaded on a DEAE column. The concentration of the flow through from the DEAE column was quantified by reverse-phase HPLC analysis using a C4 column.

Conjugation of FcF2-His to MMAE using sortase: The sortase reaction was carried out for 4 h at 37° C. with the FcF2-His immobilized on SP-sepharose resin (Cat #17072901, Cytiva); the reaction mixture contained (Gly)3-vc-PAB-MMAE and FcF2-His at a molar ratio of 20:1, and sortase-6×His and FcF2-His at a molar ratio of 1:4. After washing the SP-sepharose to remove the sortase-6×His and unreacted (Gly)3-vc-PAB-MMAE, the purified FcF2-MMAE was eluted with phosphate buffer containing 1 M NaCl. FcF2-MAME was diluted to a NaCl concentration of 200 mM before being sterilized with a 0.22 um filter and stored frozen at −80° C.

Flow cytometric analysis: Expression of LGR5 in live cells was determined by flow cytometry after staining with nti-hLGR5/GPR49 antibody at a final concentration of 12.5 ug/mL for 30 minutes at 25° C. protected from light. Excess anti-hLGR5/GPR49 antibody was removed with two rounds of PBS wash, and the cells were incubated with PE-conjugated anti-mouse IgG antibody diluted 1:20 for 30 minutes at 25° C. protected from light. Excess Anti-mouse IgG antibody was removed with two rounds of PBS wash and the cells were resuspended in 300 uL PBS with 0.5 ug/mL propidium iodide. The prepared cell suspension was analyzed on a BD FACSAria II flow cytometer.

Growth rate inhibition assay: The effect of FcF2-MMAE on cell growth rate in vitro was determined using the CCK8 reagent (Dojindo, Inc). Cells were seeded in triplicate wells for each drug concentration at densities sufficient for control wells to yield an OD450 of >1.5 after subtraction of the OD determined at the time drug exposure was started. Survival was calculated as percent reduction of the difference in the T=0 and the time the assay was stopped (Hafner et al., 2016). All data points represent the mean±SEM of triplicate cultures for each concentration of the tested drug.

Pharmacokinetic studies: BALB/c mice were injected IV with FcF2-MMAE at a dose of 0.1 nmol/g and blood was collected in EDTA-coated tubes from the cheek or tail vein at timed intervals. Plasma FcF2-MMAE concentration was determined by ELISA using capture and detection antibodies specific for human RSPO1 that recognize the Fu₁ and Fu₂ subdomains. Pharmacokinetic parameters were assessed using Phoenix WinNonlin version 8.1 (Certara Inc., Princeton, NJ, USA).

Efficacy studies: BALB/c nu/nu mice were obtained from the UCSD breeding colony and inoculated SC with tumor cells harvested from culture and mixed 2:1 vol/vol with Matrigel prior to injection of 150 μl of the mixture. The number of cells inoculated varied with the tumor type: OVCAR8/EV and OVCAR8/LGR5, 2.5×10⁶/site; KF-28, 2.5×10⁶/site; CAOV3, 5×10⁶/site. Tumor growth rate was determined from measurements of the crossed diameters measured once or twice a week using the formula V=(w²×L)/2. Control mice received vehicle alone (phosphate buffered saline with 0.02% Tween-20) on the same schedule.

### Example 3

### Inclusion of Multiple FuFu Regions in Therapeutic Compounds

Based on the hypothesis that RSPO1 could be used to target cytotoxic drugs selectively to CSC, a compound that contained 2 binding domains and armed this with the cytotoxin monomethylaurostatin (R1FF-MMAE) was generated. Since affinity of a ligand can be affected by the number of binding sites, tests were performed to see if the potency of R1FF-MMAE could be further enhanced by increasing the number of binding domains. A vector capable of expressing a protein containing 4 modified binding domains was constructed, and the FcST4-His protein was successfully produced in transiently transfected HEK293E cells and purified using nickel and ion exchange resins. Following attaching MMAE to the polypeptide using the sortase reaction, this drug (FcST4-MMAE) was characterized using gel electrophoresis, HPLC analysis on ion exchange and size exclusion columns, Western blot, and cytotoxicity testing. The results showed that FcST4-MMAE could be purified to high purity, and the compound was remarkably stable at 4° C. and in the presence of a low pH of 3.0. Cytotoxicity testing disclosed that the increase in binding domains did not enhance potency, possibly due to the increase in size of the ligand or changes in the structure mediated by inter-domain interactions that might have interfered with access to the LGR5/LGR6 receptors.

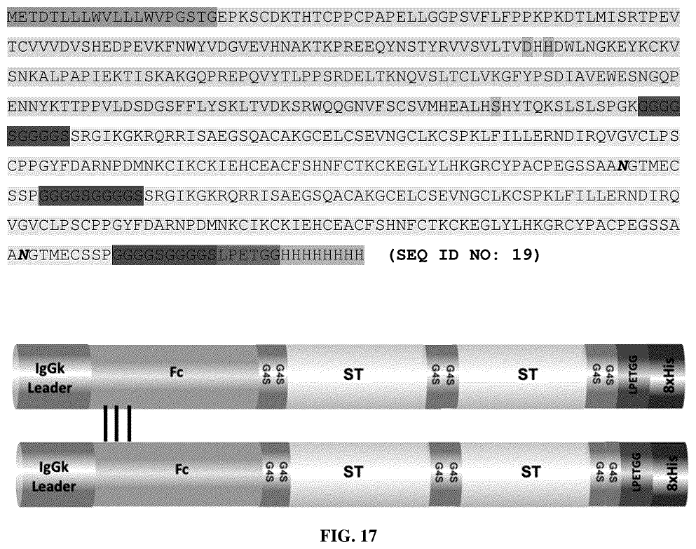

A vector construct for the FcST4 molecule was designed to contain an IgG leader sequence on the N terminal end, followed by a mutated Fc domain attached to two modified receptor binding domains (ST) in series with a linker sequence in between the two ST domains. A second spacer sequence was inserted just upstream of the LPETGG sortase recognition (donor) motif and an 8×His tag was positioned at the C terminal end. FIG. 17 shows the schematic of the steps used to create the FcST4 vector employing an overlap PCR technique, as well as the amino acid sequence for FcST4. As shown in FIG. 17, this construct contains two LGR binding domains separated by a G4S linker. Nucleotide sequence of FcST4 are provided (SEQ ID NO: 103). Restriction enzyme double digestion was used to two create two DNA fragments from an existing FcST2 vector such that both fragments had an overlap in the linker sequence in common that would allow annealing of the two fragments. Appropriate primers were designed to amplify both fragments separately first, and then the two fragments were annealed at 70° C. to generate a final product. The final product was ligated to pcDNA3.1. Sanger sequencing of a maxiprep was used to confirm that the sequence was 100% match to the planned sequence, and subsequently the ligated vector was stored at −80° C. to use for transfection.

The full-length protein was produced in HEK293e cells. The FcST4.pcDNA3.1 vector was transiently transfected in HEK293e cells. The HEK293e cells were allowed to grow in Freestyle 293 media for 5 days before being used for transfection. On the day of transfection, the cell media was refreshed and a mix containing the FcST4.pcDNA31 vector and PEI 25K (1 mg/mL) was added dropwise to the 150 mL transfection flask and incubated at 130 rpm, 5% CO2, for 4 hours. Four hours after transfection, the flask was supplemented with 150 mL of HySFM293 media and 30 mL of Freestyle 293 media to bring the final volume upto 300 mL. The cells were allowed to grow. Approximately 24 hours after transection, valproic acid and 1:1000 v/v anti-clumping agent was added to the flask containing transfected HEK293E cells. The cells were harvested on day 5. The mean cell viability on the day of harvest for the 7 batches of FcST4 produced was 79%.

After harvest, the cell supernate was processed through the initial steps of purification. The first purification step was based on the ability of the 8×His tag to bind to Ni resin. Particulate manner was removed by high-speed centrifugation of the cell supernates and then loaded on gravity flow column packed with pre-charged Ni-NTA resin. The FcST4-His was eluted using 300 mM imidazole and the protein was then passed through a DEAE column and the flow-through was characterized using HPLC reverse-phase C4 column and non-reducing SDS/PAGE gel analysis.

The results of the initial Ni purification step were examined in more detail by collecting and analyzing fractions from serial wash and elution steps with 300 mM imidazole, and the sequential flow-through and wash steps of the DEAE column. The ratio of band sizes did not vary with serial elutions indicating no selective purification of the 120 kD FcST4-His dimer could be achieved with this strategy. The DEAE step was successful in removing some of the HMW forms; an average of 28% of the FcST4-His was lost in this step. However, this loss was mostly the result of successful retention of HMW forms on the DEAE resin. Use of the serial Ni-NTA and the DEAE steps resulted in a reasonably pure form of FcST4 that then served as the input to the sortase reaction that conjugated the MMAE onto the molecule. The first step Ni-NTA purification and the second step DEAE purification resulted in a reasonably pure FcST4-His protein as evident by the presence of a single peak in the HPLC-C4 profile. The protein was successfully captured from the cell supernate by the Protein A resin, and it was successfully eluted with PBS containing 1 M NaCl as evidenced by a correct sized band on the SDS/PAGE gel.

Following the Ni-NTA and DEAE purification, the FcST4-His was linked to the cytotoxin monomethylaurostatin (MMAE) using a sortase reaction. The sortase enzyme cuts between the threonine and the glycine in the LPETGG tag removing the GG-8×His tag. The sortase reaction was carried out with the precursor FcST4-His loaded on the cation exchange resin SP-sepharose. This allowed the excess G3-val-cit-PAB-MMAE and sortase enzyme to be washed away prior to elution of the SP-sepharose with 1 M NaCl. The efficiency of the sortase reaction across all batches of FcST4-His was 100±37% (SD). However, the average percent yield of the final product was 90±22% (SD) post sortase reaction.

To assess the stability of the FcST4-MMAE molecule, the sample was subjected to multiple freeze and thaw cycles. There was no significant change in the SEC130 trace of the FcST4-MMAE molecule even after 4 cycles.

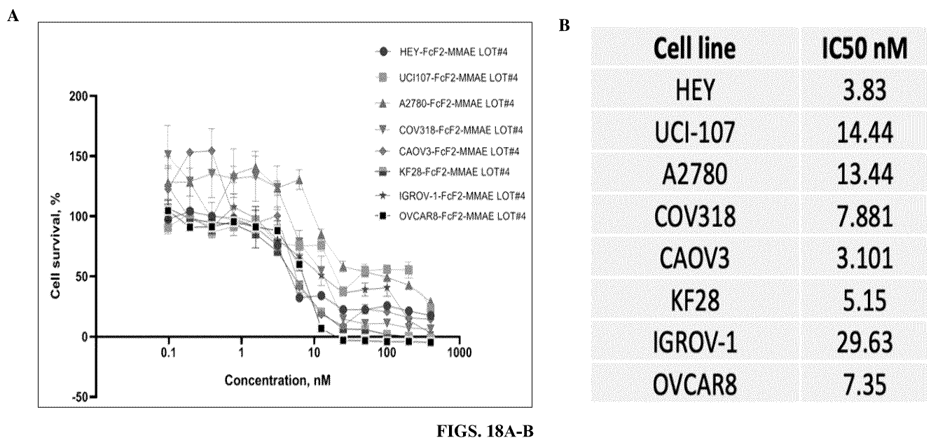

After the production of 7 batches of FcST4-MMAE, batches 2-6 were used in a cytotoxicity assay. The HEK293e suspension cells were grown and treated with the drug in a 96-well plate in a series of concentrations starting from 0 mM to 400 mM. On day 6, CCK8 reagent was added and the OD450 of the experimental plates was read. Consistently for FcST4-MMAE batches 2-6, although a difference in targeted killing was observed between HEK293e empty vector cells versus cells overexpressing LGR5 receptors, the difference was not statistically significant. The cytotoxicity assay for FcST4-MMAE was also done using the OVCAR8 cell line. Concentrations of the drug starting from 400 nM to 0 mM were tested. Similar to the results with the HEK293e cells, although a difference in targeted killing between OVCAR8 cells expressing just the empty vector and cells engineered to overexpress LGR5 receptors was observed, the difference was not statistically significant. The IC50 values ranged from 1.9 to 3.8 nM. Potency of FcST2-MMAE was tested against 8 human ovarian cancer cell lines, and results are shown in FIG. 18. FcST2-MMAE was remarkably effective at killing these cells with IC₅₀ values ranging from 3.8-29 nM and all but one cell line having an IC₅₀ of less than 20 nM.

### Example 4

### Linkerless FcF2-MMAE

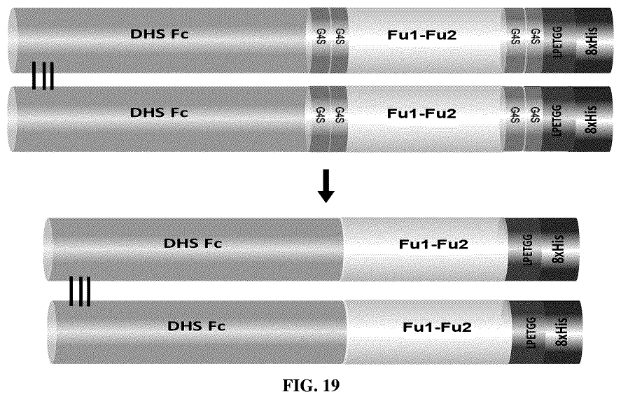

The FcF2-MMAE compound was made as described in the above Examples, with the modification that the G4S linkers were not included in the polypeptide. A diagram of the linkerless FcF2 polypeptide portion (FcF2Δlinker-His) of the compound is shown in FIG. 19.

The molecular weight of the resulting monomer was reduced by 1,279 from 42,642 to 41,363, a ˜3% decrease in size. The FcF2Δlinker-His polypeptide was produced using ExpiCHO cells. Ni-NTA and DEAE purification steps were performed. Production and purification of the FcF2Δlinker-His polypeptide was consistent with yields and purity of the FcF2-His polypeptide, based on SDS page experiments and HPLC reverse-phase C4 profiles. The MMAE was covalently attached to the FcF2Δlinker using a sortase as described in the above examples.

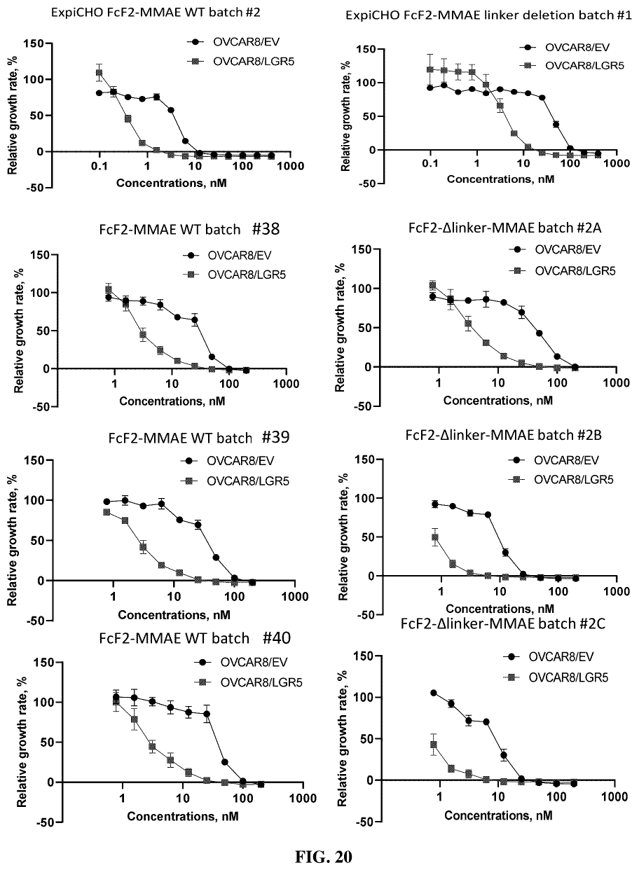

Cytotoxicity experiments were performed using FcF2-MMAE and FcF2Δlinker-MMAE on OVCAR8 cancer cells (OVCAR8/EV) and OVCAR8 cancer cells that overexpress the LGR5 receptor (OVCAR8/LGR5). The FcF2-His and FcF2Δlinker-His from which the MMAE forms were both produced in ExpiCHO. MMAE was attached using the sortase reaction described above. As shown in FIG. 20, both FcF2-MMAE and FcF2Δlinker-MMAE resulted in killing of the cancer cells, and increased cancer cell death was observed in cancer cells that overexpress the LGR5 receptor (OVCAR8/LGR5). Overall, no clear differences in IC₅₀ values or degree of selectivity were observed. FcF2Δlinker-MMAE appeared to be less potent against both LGR5-poor and LGR5-rich cells. These results indicate that therapeutic, cancer-killing effects can be observed in FcF2-MMAE constructs wherein the linkers have been excluded from the polypeptide portion of the therapeutic molecule.

### Example 5

### Efficacy of FcF2-MMAE In Vivo

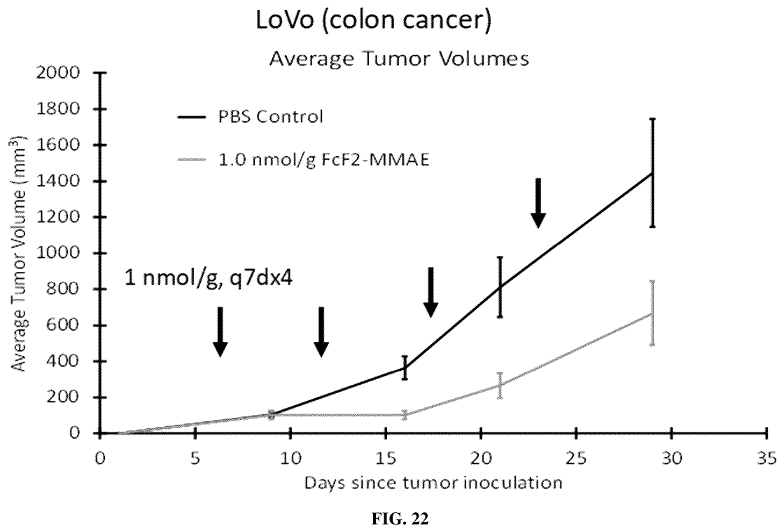

FcF2-MMAE was tested for efficacy in additional human tumor xenograft models in which tumors were grown subcutaneously from previously established tumor cell lines. The LoVo cell line is a colorectal line that contains a KRAS^{G13D} mutation and has high levels of WNT signaling. LoVo cells were inoculated SC in nu/nu mice; there were a total of 20 tumors in each group. Administration of FcF2-MMAE was started on the first day that any tumor became detectable. The control group received saline only; the experimental group received FcF2-MMAE injected IP q7dx4 at a dose of 1 nmol/g of body weight and then further administration was stopped. As shown in FIG. 22, FcF2-MMAE delayed the onset of growth and slowed the rate of grow once established relative to the tumors in the untreated control group. There was no significant weight loss in either the control or experimental groups of mice. These results demonstrate that FcF2-MMAE was active in this model. Results are shown in FIG. 21 and FIG. 22.

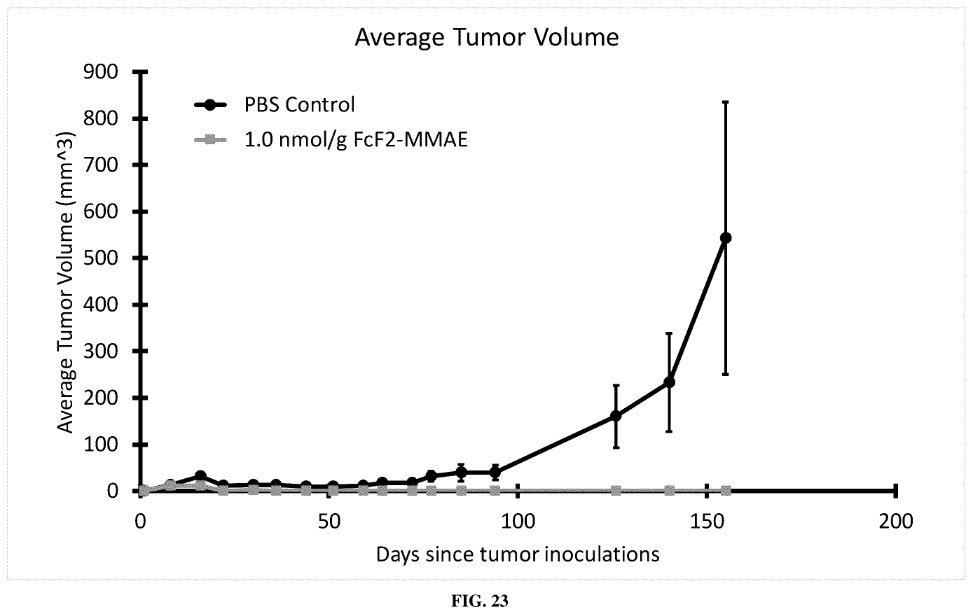

FcF2-MMAE was further tested for efficacy using tumors grown from the AGS human gastric cancer cell line. This line was selected for testing both because it has high levels of WNT pathway signaling and it is representative of a type of tumor with unmet medical need. AGS cells were inoculated SC in nu/nu mice; there were a total of 20 tumors in each group. Administration of FcF2-MMAE was started on the first day that any tumor became detectable. The control group received saline only; the experimental group received FcF2-MMAE injected IP q7dx4 at a dose of 1 nmol/g of body weight and then further administration was stopped. As shown in FIG. 23, FcF2-MMAE markedly suppressed the outgrowth of tumors in the experimental group. Among the 20 inoculations in the control group, 11 developed tumors; among the 20 inoculations in the experimental group, none developed tumors (p<0.0037 Chi-square). No tumors had appeared in the experimental group by 160 days after inoculation. There was no significant weight loss in either the control or experimental group of mice. These results demonstrate that FcF2-MMAE was curative in these mice that received FcF2-MMAE. Results are shown in FIG. 23.

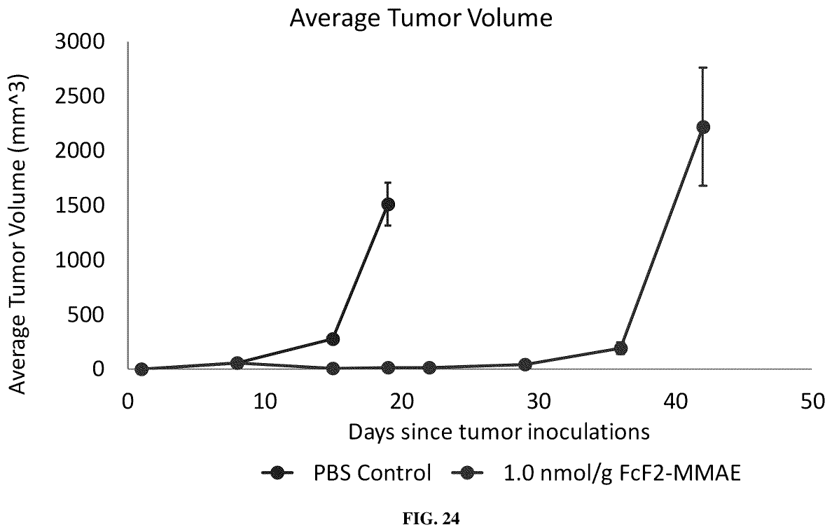

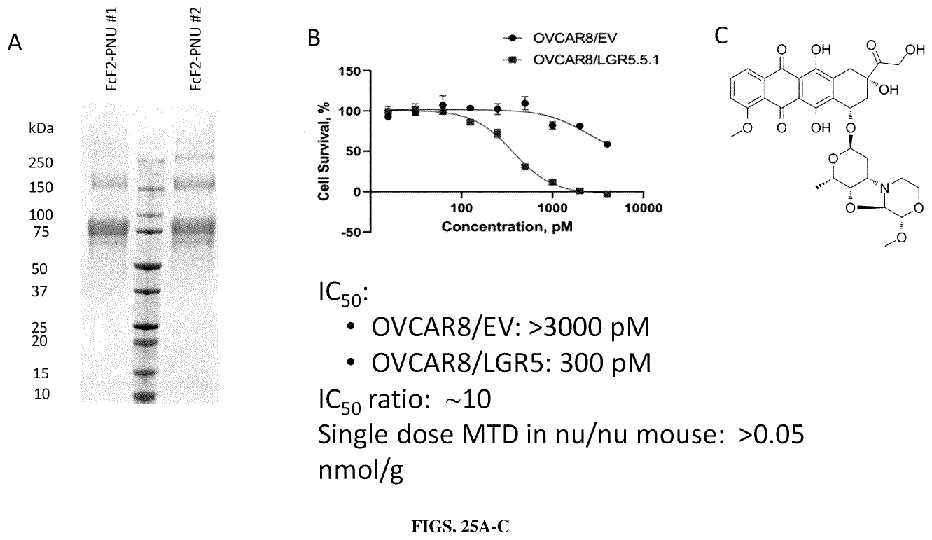

FcF2-MMAE was also tested for efficacy using tumors grown from the SKNAS neuroblastoma cell line. This line was selected for tested both because it has high levels of WNT pathway signaling and it is a very aggressive tumor. SKNAS cells were inoculated SC in nu/nu mice; there were a total of 20 tumors in each group. Administration of FcF2-MMAE was started on the first day that any tumor became detectable (day 7 after inoculation). The control group received saline only; the experimental group received FcF2-MMAE injected IP q7dx4 at a dose of 1 nmol/g of body weight and then further administration was stopped. As shown in FIG. 24, FcF2-MMAE increased the time to first detection, and increased the time until the onset of explosive tumor growth from 15 to 38 days. There was no significant weight loss in either the control or experimental group of mice. These results demonstrate that FcF2-MMAE was highly active in this model. Results are shown in FIG. 24.

### Example 6

### Conjugation of FcF2-His with Cytotoxins

As shown in this Example, FcF2-His can be conjugated to a variety of cytotoxins. While monomethylaurostatin (MMAE) is a very potent cytotoxin, even more potent cytotoxins can be used in some antibody-drug conjugates. To test and demonstrate the versatility of FcF2 as a targeting moiety, two other classes of warheads were conjugated to FcF2-His using sortase A. The same sortase A reaction conditions described above for coupling of (Gly)3-val/cit-PAB-MMAE were used for conjugating PNU159682 and deruxtecan.

FIG. 25A shows an SDS-PAGE analysis of FcF2-His conjugated with PNU159682, an anthracycline derivative. Conjugation with this molecule did not alter the structural features of FcF2-His as detected by SDS-PAGE. The FcF2-PNU159682 was tested against the LGR5-poor OVCAR8/EV cells and the OVCAR8/LGR5 cells that express approximately 10-fold more LGR5 receptors. The IC₅₀ for the OVCAR8/LGR5 cells was 300 pM whereas for the OVCAR8/EV was >3,000 pM thus yielding an IC₅₀ ratio of ˜10 (FIG. 25B). FIG. 25C shows the structure of PNU-159682.

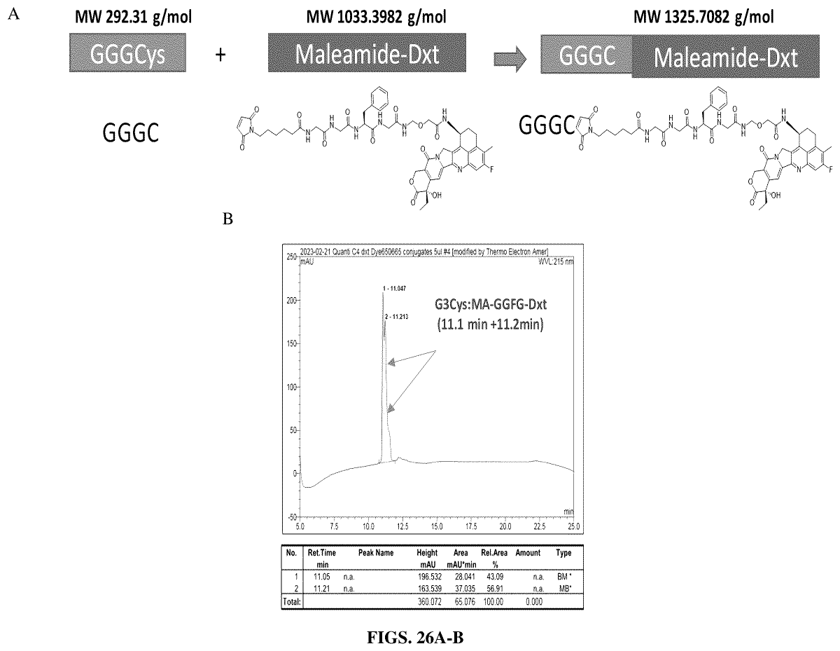

Deruxtecan has demonstrated very high efficacy in breast cancer patients as the cytotoxin included in the antibody-drug conjugate Fam-Trastuzumab Deruxtecan-nxki. To further demonstrate the feasibility of conjugating FcF2-His with other types of warheads, deruxtecan was conjugated to FcF2-His using the sortase A reaction. The first step was to construct a linker for deruxtecan that would serve as a substrate for the sortase A reaction. In order to do this, the conditions were worked out for the reaction of the peptide GGGC with commercially acquired maleimide-GGFG-deruxtecan to produce the GGGC-MA-GGFG-deruxtecan linker. This required optimization of conditions so that this linker would remain soluble in aqueous buffer. FIG. 26A shows a schematic of this reaction, and FIG. 26B shows the reverse-phase HPLC analysis of the GGGC-MA-GGFG-deruxtecan linker that documents its purity.

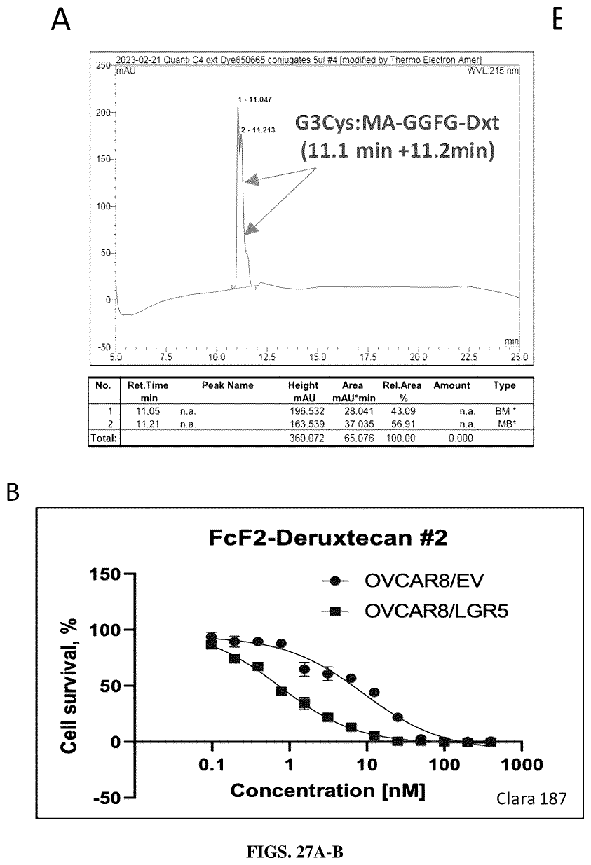

The GGGC-MA-GGFG-deruxtecan linker was conjugated to FcF2-LEPTGG-His using the sortase reaction. FIG. 27A presents a reverse-phase HPLC (C4 column) analysis of FcF2-deruxtecan that documents its purity and the absence of unreacted substrates. The ability of FcF2-derutecan to inhibit the growth of LGR5-poor OVCAR8/EV versus LGR5-rich OVCAR8/LGR5 cells was determined using the standard CCK8 cytotoxicity assay and a 120 h exposure. As shown in FIG. 27B, this assay demonstrated that FcF2-deruxtecan is very potent with IC₅₀ values of 0.7532 and 9.045 nM against the OVCAR8/LGR5 and OVCAR8/EV cells. Most importantly, FcF2-derutecan retained 12-fold selectivity in favor of the LGR5-rich cells.

Overall, these results demonstrate that FcF2-His can be successfully conjugated with a wide variety of different types of cytotoxic agents without apparent structural damage or loss of selectivity for the LGR5 stem cell receptor.

### Example 7

### Mutant Forms of FcF2-MMAE with Prolonged Plasma Half-Lives

All protein therapeutics are gradually degraded during circulation in the blood stream by plasma proteases and cellular clearance mechanisms. Western blot analysis of FcF2-MMAE incubated in human plasma at 37° C. identified a low molecular weight fragment that suggested a cut in the first part of the Fu1 domain of FcF2-MMAE. This was a region where earlier studies suggested cutting by enterokinase. Vectors expressing mutant forms of FcF2-LPETGG-His containing 4 short deletions in this area were made, and their proteins were produced and tested for rate of degradation in human plasma. This effort highlighted sub-regions of particular interest. A series of additional FcF2-LPETGG-His vectors containing single alanine substitution mutants were constructed, and their protein products produced and tested. This screen identified mutations R28A and R30A as being of particular interest, although several other single point mutants also slowed plasma degradation.

The FcF2 (R28A) mutant was generated using the nucleotide sequence:
(SEQ ID NO: 97)
GCTAGCGCCACCATGGAAACCGATACACTGCTGCTGTGGGTGCTGCTCCTTTGGGTG
CCCGGATCTACAGGCGAGCCTAAGAGCTGCGACAAGACCCACACCTGTCCTCCATG
TCCTGCTCCAGAACTGCTCGGCGGACCTTCCGTGTTCCTGTTTCCTCCAAAGCCTAAG
GACACCCTGATGATCAGCAGAACCCCTGAAGTGACCTGCGTGGTGGTGGATGTGTCC
CACGAGGATCCCGAAGTGAAGTTCAATTGGTACGTGGACGGCGTGGAAGTGCACAA
CGCCAAGACCAAGCCTAGAGAGGAACAGTACAACAGCACCTACAGAGTGGTGTCCG
TGCTGACCGTGGACCACCACGATTGGCTGAACGGCAAAGAGTACAAGTGCAAGGTG
TCCAACAAGGCCCTGCCTGCTCCTATCGAGAAAACCATCAGCAAGGCCAAGGGCCA
GCCTAGGGAACCCCAGGTTTACACACTGCCTCCAAGCAGGGACGAGCTGACCAAGA
ATCAGGTGTCCCTGACCTGCCTGGTCAAGGGCTTCTACCCTTCCGATATCGCCGTGG
AATGGGAGAGCAATGGCCAGCCTGAGAACAACTACAAGACAACCCCTCCTGTGCTG
GACAGCGACGGCTCATTCTTCCTGTACAGCAAGCTGACAGTGGACAAGAGCAGATG
GCAGCAGGGCAACGTGTTCAGCTGCAGCGTGATGCACGAAGCCCTGCACAGCCACT
ACACCCAGAAGTCCCTGTCTCTGAGCCCTGGAAAAGGCGGCGGAGGATCTGGCGGA
GGCGGATCTTCTAGAGGCATCAAGGGCAAGGCCCAGCGGAGAATCTCTGCCGAGGG
ATCTCAGGCCTGTGCCAAGGGCTGTGAACTGTGCAGCGAAGTGAACGGCTGCCTGA
AGTGCAGCCCCAAGCTGTTCATCCTGCTGGAACGGAACGACATCCGGCAAGTGGGA
GTGTGCCTGCCTTCTTGTCCTCCTGGCTACTTCGACGCCAGAAATCCCGACATGAAC
AAGTGCATTAAGTGCAAGATCGAGCACTGCGAGGCCTGCTTCAGCCACAACTTCTGC
ACCAAGTGCAAAGAGGGCCTGTACCTGCACAAGGGCAGATGCTATCCTGCCTGTCC
TGAGGGAAGCTCTGCCGCCAATGGCACAATGGAATGTAGCTCTCCAGGTGGCGGAG
GAAGCGGTGGCGGCGGATCTCTTCCAGAAACAGGCGGACACCATCACCATCATCAC
CACCACTGATGACTCGAG
The FcF2 (R28A) mutant as the amino acid sequence:
(SEQ ID NO: 98)
ASATMETDTLLLWVLLLWVPGSTGEPKSCDKTHTCPPCPAPELLGGPSVFLFPPKPKDTL
MISRTPEVTCVVVDVSHEDPEVKFNWYVDGVEVHNAKTKPREEQYNSTYRVVSVLTV
DHHDWLNGKEYKCKVSNKALPAPIEKTISKAKGQPREPQVYTLPPSRDELTKNQVSLTC
LVKGFYPSDIAVEWESNGQPENNYKTTPPVLDSDGSFFLYSKLTVDKSRWQQGNVFSCS
VMHEALHSHYTQKSLSLSPGKGGGGSGGGGSSRGIKGKAQRRISAEGSQACAKGCELCS
EVNGCLKCSPKLFILLERNDIRQVGVCLPSCPPGYFDARNPDMNKCIKCKIEHCEACFSH
NFCTKCKEGLYLHKGRCYPACPEGSSAANGTMECSSPGGGGSGGGGSLPETGGHHHHH
HHH--
The Ful-Fu2
(R28A) mutant has the amino acid sequence:
(SEQ ID NO: 90)
SRGIKGKAQRRISAEGSQACAKGCELCSEVNGCLKCSPKLFILLERNDIRQVGVCLPSCPP
GYFDARNPDMNKCIKCKIEHCEACFSHNFCTKCKEGLYLHKGRCYPACPEGSSAANGT
MECSSP
The FcF2 (R30A) mutant was generated using the nucleotide sequence:
(SEQ ID NO: 99)
GCTAGCGCCACCATGGAAACCGATACACTGCTGCTGTGGGTGCTGCTCCTTTGGGTG
CCCGGATCTACAGGCGAGCCTAAGAGCTGCGACAAGACCCACACCTGTCCTCCATG
TCCTGCTCCAGAACTGCTCGGCGGACCTTCCGTGTTCCTGTTTCCTCCAAAGCCTAAG
GACACCCTGATGATCAGCAGAACCCCTGAAGTGACCTGCGTGGTGGTGGATGTGTCC
CACGAGGATCCCGAAGTGAAGTTCAATTGGTACGTGGACGGCGTGGAAGTGCACAA
CGCCAAGACCAAGCCTAGAGAGGAACAGTACAACAGCACCTACAGAGTGGTGTCCG
TGCTGACCGTGGACCACCACGATTGGCTGAACGGCAAAGAGTACAAGTGCAAGGTG
TCCAACAAGGCCCTGCCTGCTCCTATCGAGAAAACCATCAGCAAGGCCAAGGGCCA
GCCTAGGGAACCCCAGGTTTACACACTGCCTCCAAGCAGGGACGAGCTGACCAAGA
ATCAGGTGTCCCTGACCTGCCTGGTCAAGGGCTTCTACCCTTCCGATATCGCCGTGG
AATGGGAGAGCAATGGCCAGCCTGAGAACAACTACAAGACAACCCCTCCTGTGCTG
GACAGCGACGGCTCATTCTTCCTGTACAGCAAGCTGACAGTGGACAAGAGCAGATG
GCAGCAGGGCAACGTGTTCAGCTGCAGCGTGATGCACGAAGCCCTGCACAGCCACT
ACACCCAGAAGTCCCTGTCTCTGAGCCCTGGAAAAGGCGGCGGAGGATCTGGCGGA
GGCGGATCTTCTAGAGGCATCAAGGGCAAGAGACAGGCCAGAATCTCTGCCGAGGG
ATCTCAGGCCTGTGCCAAGGGCTGTGAACTGTGCAGCGAAGTGAACGGCTGCCTGA
AGTGCAGCCCCAAGCTGTTCATCCTGCTGGAACGGAACGACATCCGGCAAGTGGGA
GTGTGCCTGCCTTCTTGTCCTCCTGGCTACTTCGACGCCAGAAATCCCGACATGAAC
AAGTGCATTAAGTGCAAGATCGAGCACTGCGAGGCCTGCTTCAGCCACAACTTCTGC
ACCAAGTGCAAAGAGGGCCTGTACCTGCACAAGGGCAGATGCTATCCTGCCTGTCC
TGAGGGAAGCTCTGCCGCCAATGGCACAATGGAATGTAGCTCTCCAGGTGGCGGAG
GAAGCGGTGGCGGCGGATCTCTTCCAGAAACAGGCGGACACCATCACCATCATCAC
CACCACTGATGACTCGAG
The FcF2 (R30A) mutant has the amino acid sequence:
(SEQ ID NO: 98)
ASATMETDTLLLWVLLLWVPGSTGEPKSCDKTHTCPPCPAPELLGGPSVFLFPPKPKDTL
MISRTPEVTCVVVDVSHEDPEVKFNWYVDGVEVHNAKTKPREEQYNSTYRVVSVLTV
DHHDWLNGKEYKCKVSNKALPAPIEKTISKAKGQPREPQVYTLPPSRDELTKNQVSLTC
LVKGFYPSDIAVEWESNGQPENNYKTTPPVLDSDGSFFLYSKLTVDKSRWQQGNVFSCS
VMHEALHSHYTQKSLSLSPGKGGGGSGGGGSSRGIKGKRQARISAEGSQACAKGCELCS
EVNGCLKCSPKLFILLERNDIRQVGVCLPSCPPGYFDARNPDMNKCIKCKIEHCEACFSH
NFCTKCKEGLYLHKGRCYPACPEGSSAANGTMECSSPGGGGSGGGGSLPETGGHHHHH
HHH
The Fu1-Fu2 (R30A) mutant has the nucleotide sequence:
(SEQ ID NO: 100)
TCTAGAGGCATCAAGGGCAAGAGACAGGCCAGAATCTCTGCCGAGGGATCTCAGGC
CTGTGCCAAGGGCTGTGAACTGTGCAGCGAAGTGAACGGCTGCCTGAAGTGCAGCC
CCAAGCTGTTCATCCTGCTGGAACGGAACGACATCCGGCAAGTGGGAGTGTGCCTG
CCTTCTTGTCCTCCTGGCTACTTCGACGCCAGAAATCCCGACATGAACAAGTGCATT
AAGTGCAAGATCGAGCACTGCGAGGCCTGCTTCAGCCACAACTTCTGCACCAAGTG
CAAAGAGGGCCTGTACCTGCACAAGGGCAGATGCTATCCTGCCTGTCCTGAGGGAA
GCTCTGCCGCCAATGGCACAATGGAATGTAGCTCTCCA

Variants of the FcF2 construct were generated using Fu1-Fu2 regions that contain additional deletion mutations, as follows. Additional deletion mutants of the Fu1-Fu2 region were generated: R22-R31 (RSPO1 nucleotides 64 to 93), K25-R31 (RSPO1 nucleotides 73 to 93), R28-R31 (RSPO1 nucleotides 82-93), and R22-K27 (RSPO1 nucleotides 64-81). These deletion mutants can be used in a similar fashion to other Fu1-Fu2 regions to bind LGR. The deletion mutants have the following sequences: Fu1-Fu2 (R22-R31 deletion)

(SEQ ID NO: 92)
SISAEGSQACAKGCELCSEVNGCLKCSPKLFILLERNDIRQVGVCLPSC
PPGYFDARNPDMNKCIKCKIEHCEACFSHNFCTKCKEGLYLHKGRCYPA
CPEGSSAANGTMECSSP,
Fu1-Fu2 (K25-R31 deletion)
(SEQ ID NO: 93)
SRGIISAEGSQACAKGCELCSEVNGCLKCSPKLFILLERNDIRQVGVCL
PSCPPGYFDARNPDMNKCIKCKIEHCEACFSHNFCTKCKEGLYLHKGRC
YPACPEGSSAANGTMECSSP,
Ful-Fu2 (R28-R31 deletion)
(SEQ ID NO: 94)
SRGIKGKISAEGSQACAKGCELCSEVNGCLKCSPKLFILLERNDIRQVG
VCLPSCPPGYFDARNPDMNKCIKCKIEHCEACFSHNFCTKCKEGLYLHK
GRCYPACPEGSSAANGTMECSSP,
Ful-Fu2 (R22-K27 deletion)
(SEQ ID NO: 95)
SRQRRISAEGSQACAKGCELCSEVNGCLKCSPKLFILLERNDIRQVGVC
LPSCPPGYFDARNPDMNKCIKCKIEHCEACFSHNFCTKCKEGLYLHKGR
CYPACPEGSSAANGTMECSSP,
and
Ful-Fu2 (S21-Q38 deletion)
(SEQ ID NO: 96)
ACAKGCELCSEVNGCLKCSPKLFILLERNDIRQVGVCLPSCPPGYFDAR
NPDMNKCIKCKIEHCEACFSHNFCTKCKEGLYLHKGRCYPACPEGSSAA
NGTMECSSP.

These deletion mutants may result in an increase in plasma half-life.

FcF2-MMAE (WT), FcF2-R28A-MMAE and FcF2-R30A-MMAE were tested for potency and selectivity using the OVCAR8/EV versus OVCAR8/LGR5 cells. FIG. 28 shows data of the concentration-survival curves, and it was observed that neither the R28A nor the R30A mutation altered potency of selectivity for LGR5-rich cells.

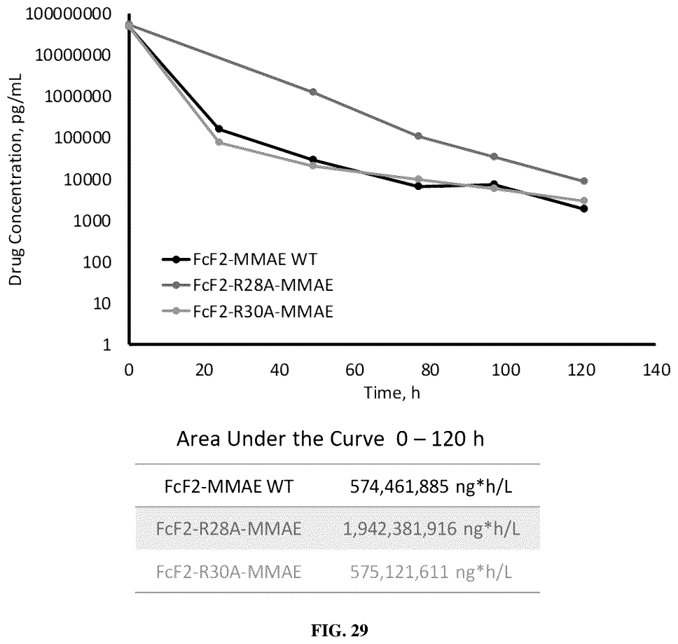

Pharmacokinetic studies were then performed in BALB/c mice. Mice were given an intravenous (IV) injection of either FcF2-MMAE (WT), FcF2-R28A-MMAE, or FcF2-R30A-MMAE and plasma concentration of each was determined in timed samples by ELISA using capture and detection antibodies to different parts of the molecule. FIG. 29 shows that the R28A, but not the R30A, mutation prolonged the initial half-life of the molecule and increased the AUC₀₋₁₂₀ by a factor of 3.4-fold. Of particular importance is that the concentration of FcF2-R28A-MMAE was greater than two (>2) orders of magnitude higher than that of FcF2-MAME during the early phase of the plasma decay curve when drug penetration into tumor is likely to be occurring at the highest rate. These results demonstrate that the R28A mutation can increase the half-life of the FcF2 construct comprising a cytotoxic construct.

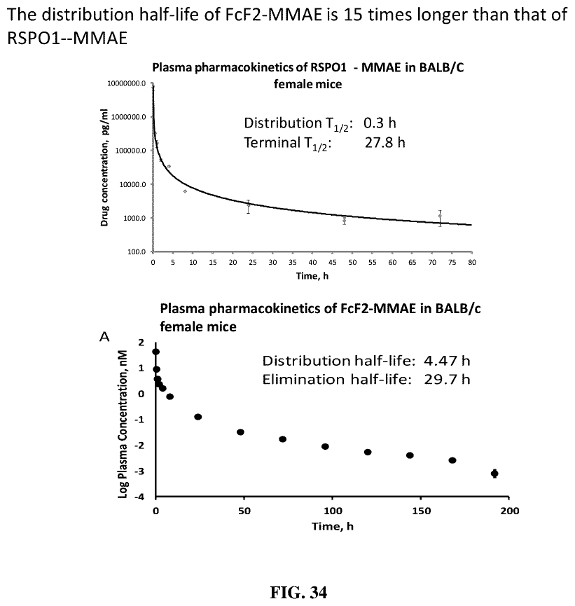

The plasma half-life of FcF2-MMAE construct was compared to the R1FF-MMAE (“RSPO1-MMAE”) construct described in Yu et al. (2021) using BALB/C female mice. The in vivo distribution half-life of FcF2-MMAE was observed to be about 15 times longer than that of R1FF-MMAE. Results are shown in FIG. 34.

### Example 8

### Increased Loading of MMAE onto FcF2-his Using Sortase E

Some studies using antibody-drug conjugates support the idea that, in some cases, an increase in number of cytotoxins per antibody molecule (DAR) can increase potency and efficacy. In the case of FcF2-LPETGG-His, the sortase reaction can put MMAE warheads on both ends of the molecule. The main challenge is that the sortase A reaction is reversible so that, in the process of adding a second MMAE to the N-ter end, it may remove and MMAE already loaded on the C-ter end. The inventors then worked to develop a further method of attaching an additional cytotoxic moiety to FcF2.

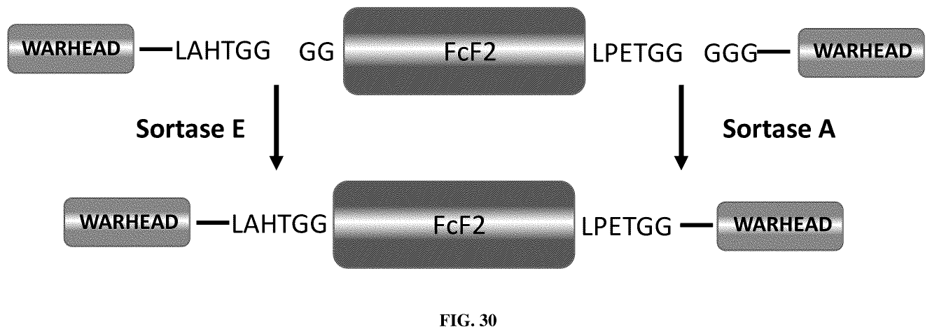

FIG. 30 presents a schematic of the approach. The recently-isolated sortase E links substrates containing an N-terminal GG motif to the sequence LAHTGG (SEQ ID NO: 106) on the C-ter end of other proteins or peptides. Since sortase A and sortase E have high specificity for different recognition sequences (LPETGGG versus LAHTGG), operating together there is the potential to load MMAE on both ends of a variant of the FcF2 molecule that contains a diglycine at the N-terminal end and a LPETGG sequence at the C-terminal end.

A vector that expresses GG-FcF2-LEPTGG-His was constructed, produced in HEK293 cells, and purified; testing showed that when loaded with MMAE using the sortase A, the addition of two glycines at the N-ter end did not alter potency or selectivity.

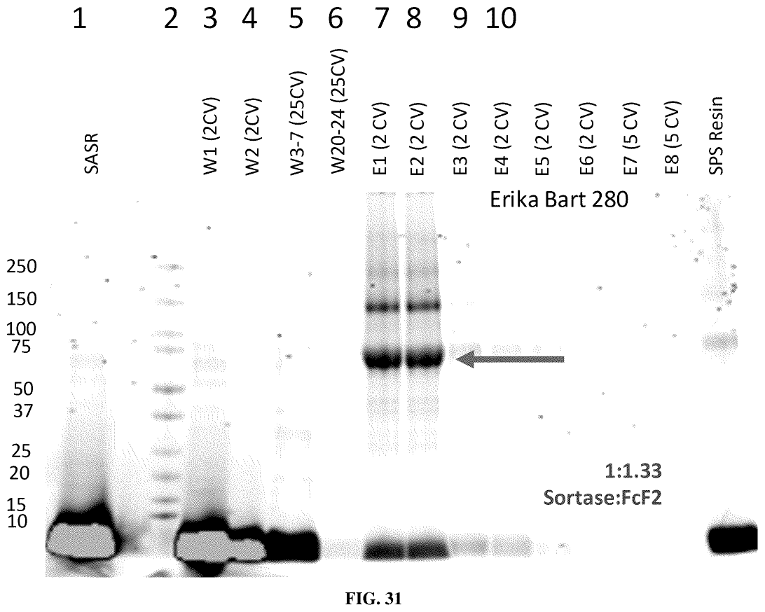

A gene containing the sortase E sequence was constructed and cloned into a bacterial expression vector and the protein was synthesized in the Rosetta strain of E. coli and purified using Ni-NTA chromatography. To facilitate the testing of sortase E, we constructed a version of the FcF2-LPETGG-His vector in which the LPETGG sequence was exchanged for the sortase E recognition sequence LAHTGG (SEQ ID NO: 106). This protein was produced and purified from HEK293 cells. As a test substrate for the sortase E reaction, the inventors prepared a linker consisting of the peptide GGGC conjugated with a maleimide-containing fluorescent molecule, Dye650. FIG. 31 shows that sortase E was able to load GGGC-MA-Dye650 onto FcF2-LAHTGG-His, thus establishing that both sortase A and sortase E can be used to load substrates with N-terminal glycines onto FcF2 containing the appropriate sortase recognition sequence. Repeating these experiments will further support and, if desired, can be used to perform statistical analysis for these results.

### Example 9

### Increased Loading of MMAE onto FcF2-his Using Partial Reduction of Disulfides

The FcF2-LEPTGG-His molecule contains 3 disulfide bonds in the immunoglobulin Fc domain and 8 in the Fu1-Fu2 domains. Antibody-drug conjugates are often loaded with cytotoxins by partial reduction of their Fc domain disulfide bonds followed by reaction with a linker containing a thiol-reactive maleimide group previously coupled to the cytotoxin. This allows loading of 2-8 or more cytotoxins per molecule but must be individualized as excess loading can distort protein structure and reduce plasma half-lives. To determine whether this same approach could be used to load MMAE onto FcF2-His without use of the sortase reaction, a series of experiments was performed assessing loading as a function of TCEP concentration.

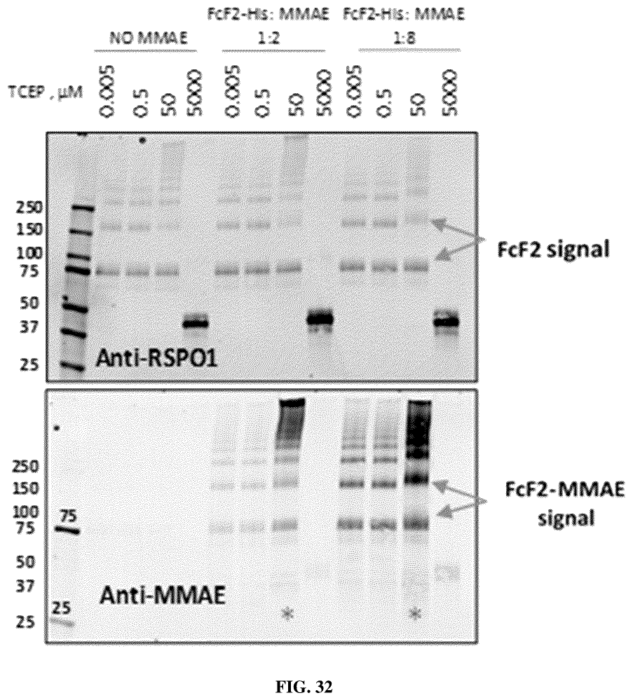

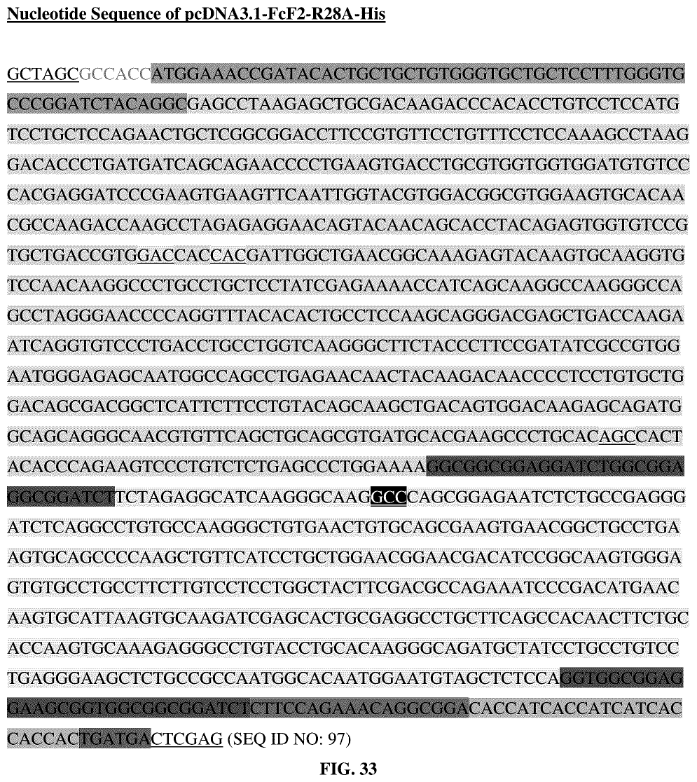

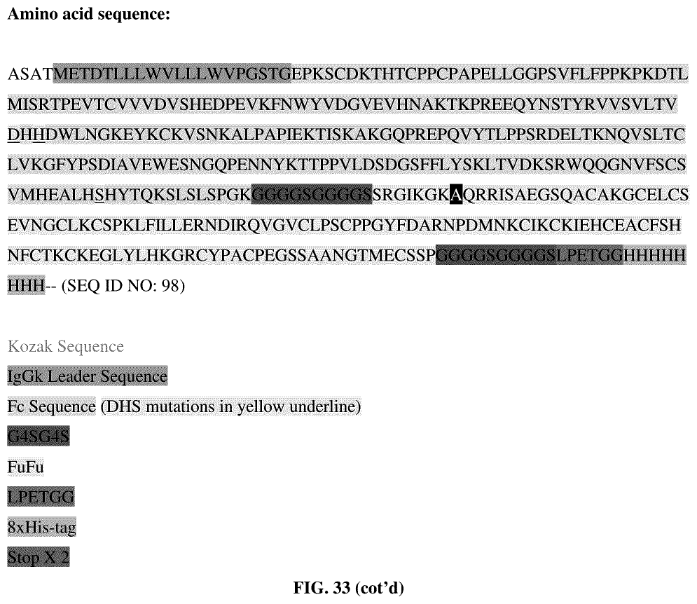

Aliquots of FcF2-His were exposed to TCEP at concentrations from 0.0005 to 5000 μM and 25 min later maleimide-val/cit-PAB-MMAE (MA-MMAE) was added to the reaction at ratios of either 1:2 or 1:8 (protein:MMAE). The reaction was then allowed to continue overnight. FIG. 32 shows the Western blot analysis of these samples probed with anti-RSPO1 and anti-MMAE. In the absence of MA-MMAE no MMAE signal was detectable; however, increasing concentrations of TCEP resulted in progressively larger and larger amounts of MMAE being loaded onto FcF2-His. The higher protein:MA-MMAE ratio resulted in higher loading. A concentration of 5000 uM TCEP reduced all disulfide bonds so that only FcF2-His monomers are visible, but when all disulfides were reduced a very large amount of MMAE was loaded.

This data demonstrates that the approach of partial disulfide reduction can be used to load MMAE on FcF2-His. Additional studies can be performed to measure the number of molecules of MMAE loaded, and how efficacy, selectivity and plasma half-life vary with loading. Increased loading of the cytotoxic moiety (e.g., MMAE) can result in increased potency or enhanced killing of the construct due to the additional molecules of the cytotoxic moiety. As shown above, covalent bonding of additional cytotoxic moieties to the construct (e.g., on the N-terminal and C-terminal ends of FcF2) can be covalently attached via a variety of techniques, including partial reduction of disulfides and covalent attachment with sortase (e.g., sortase A and sortase E).

All of the methods disclosed and claimed herein can be made and executed without undue experimentation in light of the present disclosure. While the compositions and methods of this invention have been described in terms of preferred embodiments, it will be apparent to those of skill in the art that variations may be applied to the methods and in the steps or in the sequence of steps of the method described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

### REFERENCES

The following references, to the extent that they provide exemplary procedural or other details supplementary to those set forth herein, are specifically incorporated herein by reference.
WO 2013003555,
U.S. Pat. No. 7,238,489
U.S. Patent Application 2014/0030697
Chen, Hao et al. “Tubulin Inhibitor-Based Antibody-Drug Conjugates for Cancer Therapy.” Molecules (Basel, Switzerland) vol. 22, 8 1281. 1 Aug. 2017.
Kostova, Vesela et al. “The Chemistry Behind ADCs.” Pharmaceuticals (Basel, Switzerland) vol. 14, 5 442. 7 May. 2021.
Lambert, John M, and Charles Q Morris. “Antibody-Drug Conjugates (ADCs) for Personalized Treatment of Solid Tumors: A Review.” Advances in therapy vol. 34, 5: 1015-1035; 2017.
Popp M W, Ploegh H L (2011) Making and Breaking Peptide Bonds: Protein Engineering Using Sortase. Angew Chem Int Ed 50:5024-5032;
Guimaraes C P et al. (2011) Identification of host cell factors required for intoxication through use of modified cholera toxin. J Cell Biol 195:751-764;
Popp M W, Antos J M, Grotenbreg G M, Spooner E, Ploegh H L (2007) Sortagging: a versatile method for protein labeling. Nat Chem Biol 3:707-708.
McClanahan T, et al. Identification of overexpression of orphan G protein-coupled receptor GPR49 in human colon and ovarian primary tumors. Cancer Biol Ther. 5(4):419-26, 2006.
Cortesi E, Ventura J J. Lgr6: From Stemness to Cancer Progression. J Lung Health Dis. 2019 Jun. 18; 3(1):12-15, 2019.
De Lau, W., The R-spondins protein family, Genome Biology, 13:242, 2012
Yu Z, Pestell T G, Lisanti M P, Pestell R G. Cancer stem cells. Int J Biochem Cell Biol. (2012) December; 44(12):2144-51.
Azhdarinia A, Voss J, Ghosh S C, Simien J A, Hernandez Vargas S, Cui J, Yu W A, Liu Q and Carmon K S (2018) Evaluation of Anti-LGR5 Antibodies by ImmunoPET for Imaging Colorectal Tumors and Development of Antibody-Drug Conjugates. Mol Pharm 15:2448-2454.
Azkanaz M, Corominas-Murtra B, Ellenbroek S I J, Bruens L, Webb A T, Laskaris D, Oost K C, Lafirenze S J A, Annusver K, Messal H A, Iqbal S, Flanagan D J, Huels D J, Rojas-Rodriguez F, Vizoso M, Kasper M, Sansom O J, Snippert H J, Liberali P, Simons B D, Katajisto P, Hannezo E and van Rheenen J (2022) Retrograde movements determine effective stem cell numbers in the intestine. Nature 607:548-554.
Barker N, Huch M, Kujala P, van de Wetering M, Snippert H J, van Es J H, Sato T, Stange D E, Begthel H, van den Born M, Danenberg E, van den Brink S, Korving J, Abo A, Peters P J, Wright N, Poulsom R and Clevers H (2010) Lgr5(+ve) stem cells drive self-renewal in the stomach and build long-lived gastric units in vitro. Cell Stem Cell 6:25-36.
Carmon K S, Gong X, Lin Q, Thomas A and Liu Q (2011) R-spondins function as ligands of the orphan receptors LGR4 and LGR5 to regulate Wnt/beta-catenin signaling. Proc Natl Acad Sci USA 108:11452-11457.
Clarke M F (2019) Clinical and Therapeutic Implications of Cancer Stem Cells. N Engl J Med 380:2237-2245.
Cui J, Toh Y, Park S, Yu W, Tu J, Wu L, Li L, Jacob J, Pan S, Carmon K S and Liu Q J (2021) Drug Conjugates of Antagonistic R-Spondin 4 Mutant for Simultaneous Targeting of Leucine-Rich Repeat-Containing G Protein-Coupled Receptors 4/5/6 for Cancer Treatment. J Med Chem.
de Lau W, Peng W C, Gros P and Clevers H (2014) The R-spondin/Lgr5/Rnf43 module: regulator of Wnt signal strength. Genes Dev 28:305-316.
de Vreede G, Gerlach S U and Bilder D (2022) Epithelial monitoring through ligand-receptor segregation ensures malignant cell elimination. Science 376:297-301.
Dubey R, van Kerkhof P, Jordens I, Malinauskas T, Pusapati G V, McKenna J K, Li D, Carette J E, Ho M, Siebold C, Maurice M, Lebensohn A M and Rohatgi R (2020) R-spondins engage heparan sulfate proteoglycans to potentiate WNT signaling. eLife 9.
Gong X, Azhdarinia A, Ghosh S C, Xiong W, An Z, Liu Q and Carmon K S (2016) LGR5-Targeted Antibody-Drug Conjugate Eradicates Gastrointestinal Tumors and Prevents Recurrence. Mol Cancer Ther 15:1580-1590.
Gupta P B, Pastushenko I, Skibinski A, Blanpain C and Kuperwasser C (2019) Phenotypic Plasticity: Driver of Cancer Initiation, Progression, and Therapy Resistance. Cell Stem Cell 24:65-78.
Hafner M, Niepel M, Chung M and Sorger P K (2016) Growth rate inhibition metrics correct for confounders in measuring sensitivity to cancer drugs. Nature methods 13:521-527.
Junttila M R, Mao W, Wang X, Wang B E, Pham T, Flygare J, Yu S F, Yee S, Goldenberg D, Fields C, Eastham-Anderson J, Singh M, Vij R, Hongo J A, Firestein R, Schutten M, Flagella K, Polakis P and Polson A G (2015) Targeting LGR5+ cells with an antibody-drug conjugate for the treatment of colon cancer. Science translational medicine 7:314ra186.
Kessler M, Hoffmann K, Brinkmann V, Thieck O, Jackisch S, Toelle B, Berger H, Mollenkopf H J, Mangler M, Sehouli J, Fotopoulou C and Meyer T F (2015) The Notch and Wnt pathways regulate stemness and differentiation in human fallopian tube organoids. Nature communications 6:8989.
Kim H, Lee D H, Park E, Myung J K, Park J H, Kim D I, Kim S I, Lee M, Kim Y, Park C M, Hyun C L, Maeng Y H, Lee C and Jang B (2022) Differential epithelial and stromal LGR5 expression in ovarian carcinogenesis. Scientific reports 12:11200.
Kim K A, Kakitani M, Zhao J, Oshima T, Tang T, Binnerts M, Liu Y, Boyle B, Park E, Emtage P, Funk W D and Tomizuka K (2005) Mitogenic influence of human R-spondin1 on the intestinal epithelium. Science 309:1256-1259.
Lebensohn A M and Rohatgi R (2018) R-spondins can potentiate WNT signaling without LGRs. eLife 7.
Lee C H, Kang T H, Godon O, Watanabe M, Delidakis G, Gillis C M, Sterlin D, Hardy D, Cogne M, Macdonald L E, Murphy A J, Tu N, Lee J, McDaniel J R, Makowski E, Tessier P M, Meyer A S, Bruhns P and Georgiou G (2019) An engineered human Fc domain that behaves like a pH-toggle switch for ultra-long circulation persistence. Nature communications 10:5031.
Lee C H, Kang T H, Godon O, Watanabe M, Delidakis G, Gillis C M, Sterlin D, Hardy D, Cogné M, Macdonald L E, Murphy A J, Tu N, Lee J, McDaniel J R, Makowski E, Tessier P M, Meyer A S, Bruhns P, Georgiou G. (2019) An engineered human Fc domain that behaves like a pH-toggle switch for ultra-long circulation persistence. Nat Commun. November 6; 10(1):5031.
Lee S, Jun J, Kim W J, Tamayo P and Howell S B (2020) WNT Signaling Driven by R-spondin 1 and LGR6 in High-grade Serous Ovarian Cancer. Anticancer Res 40:6017-6028.
Peng W C, de Lau W, Madoori P K, Forneris F, Granneman J C, Clevers H and Gros P (2013) Structures of Wnt-antagonist ZNRF3 and its complex with R-spondin 1 and implications for signaling. PLoS One 8:e83110.
Raslan A A and Yoon J K (2019) R-spondins: Multi-mode WNT signaling regulators in adult stem cells. Int J Biochem Cell Biol 106:26-34.
Sato T, Vries R G, Snippert H J, van de Wetering M, Barker N, Stange D E, van Es J H, Abo A, Kujala P, Peters P J and Clevers H (2009) Single Lgr5 stem cells build crypt-villus structures in vitro without a mesenchymal niche. Nature 459:262-2655.
Schindler A J, Watanabe A and Howell S B (2017) LGR5 and LGR6 in stem cell biology and ovarian cancer. Oncotarget 9:1346-1355.
Shim H (2020) Bispecific Antibodies and Antibody-Drug Conjugates for Cancer Therapy: Technological Considerations. Biomolecules 10.
Sun T, Annunziato S, Bergling S, Sheng C, Orsini V, Forcella P, Pikiolek M, Kancherla V, Holwerda S, Imanci D, Wu F, Meylan L C, Puehringer L F, Waldt A, Oertli M, Schuierer S, Terracciano L M, Reinker S, Ruffner H, Bouwmeester T, Sailer A W, George E, Roma G, de Weck A, Piscuoglio S, Lohmann F, Naumann U, Liberali P, Cong F and Tchorz J S (2021) ZNRF3 and RNF43 cooperate to safeguard metabolic liver zonation and hepatocyte proliferation. Cell Stem Cell.
Tian H, Biehs B, Warming S, Leong K G, Rangell L, Klein O D and de Sauvage F J (2011) A reserve stem cell population in small intestine renders Lgr5-positive cells dispensable. Nature 478:255-259.
Weber E L, Lai Y C, Lei M, Jiang T X and Chuong C M (2020) Human Fetal Scalp Dermal Papilla Enriched Genes and the Role of R-Spondin-1 in the Restoration of Hair Neogenesis in Adult Mouse Cells. Frontiers in cell and developmental biology 8:583434.
Xie Y, Zamponi R, Charlat O, Ramones M, Swalley S, Jiang X, Rivera D, Tschantz W, Lu B, Quinn L, Dimitri C, Parker J, Jeffery D, Wilcox S K, Watrobka M, LeMotte P, Granda B, Porter J A, Myer V E, Loew A and Cong F (2013) Interaction with both ZNRF3 and LGR4 is required for the signalling activity of R-spondin. EMBO Rep 14:1120-1126.
Yan K S, Janda C Y, Chang J, Zheng G X Y, Larkin K A, Luca V C, Chia L A, Mah A T, Han A, Terry J M, Ootani A, Roelf K, Lee M, Yuan J, Li X, Bolen C R, Wilhelmy J, Davies P S, Ueno H, von Furstenberg R J, Belgrader P, Ziraldo S B, Ordonez H, Henning S J, Wong M H, Snyder M P, Weissman I L, Hsueh A J, Mikkelsen T S, Garcia K C and Kuo C J (2017) Non-equivalence of Wnt and R-spondin ligands during Lgr5+ intestinal stem-cell self-renewal. Nature 545:238-242.
Yu S, Mulero M C, Chen W, Shang X, Tian S, Watanabe J, Watanabe A, Vorberg T, Wong C, Gately D and Howell S B (2021) Therapeutic targeting of tumor cells rich in LGR stem cell receptors. Bioconjugate Chemistry 32:376-384.
Zebisch M, Xu Y, Krastev C, MacDonald B T, Chen M, Gilbert R J, He X and Jones E Y (2013) Structural and molecular basis of ZNRF3/RNF43 transmembrane ubiquitin ligase inhibition by the Wnt agonist R-spondin. Nature communications 4:2787.
Zhang S, Dolgalev I, Zhang T, Ran H, Levine D A and Neel B G (2019) Both fallopian tube and ovarian surface epithelium are cells-of-origin for high-grade serous ovarian carcinoma. Nature communications 10:5367.
Zhang Z, Broderick C, Nishimoto M, Yamaguchi T, Lee S J, Zhang H, Chen H, Patel M, Ye J, Ponce A, Brady J, Baribault H, Li Y and Yeh W C (2020) Tissue-targeted R-spondin mimetics for liver regeneration. Scientific reports 10:13951.
Zhao J, de Vera J, Narushima S, Beck E X, Palencia S, Shinkawa P, Kim K A, Liu Y, Levy M D, Berg D J, Abo A and Funk W D (2007) R-spondin1, a novel intestinotrophic mitogen, ameliorates experimental colitis in mice. Gastroenterology 132:1331-1343.
Zhao J, Kim K A, De Vera J, Palencia S, Wagle M and Abo A (2009) R-Spondin1 protects mice from chemotherapy or radiation-induced oral mucositis through the canonical Wnt/beta-catenin pathway. Proc Natl Acad Sci USA 106:2331-2336.
Zhou W J, Geng Z H, Spence J R and Geng J G (2013) Induction of intestinal stem cells by R-spondin 1 and Slit2 augments chemoradioprotection. Nature 501:107-111.

Sequences

FcF2 sequence-without leader sequence or 8x His (SEQ ID NO: 1)
EPKSCDKTHTCPPCPAPELLGGPSVFLFPPKPKDTLMISRTPEVTCVVVDVSHEDPEVKF
NWYVDGVEVHNAKTKPREEQYNSTYRVVSVLTVDHHDWLNGKEYKCKVSNKALPAPI
EKTISKAKGQPREPQVYTLPPSRDELTKNQVSLTCLVKGFYPSDIAVEWESNGQPENNY
KTTPPVLDSDGSFFLYSKLTVDKSRWQQGNVFSCSVMHEALHSHYTQKSLSLSPGKGG
GGSGGGGSSRGIKGKRQRRISAEGSQACAKGCELCSEVNGCLKCSPKLFILLERNDIRQV
GVCLPSCPPGYFDARNPDMNKCIKCKIEHCEACFSHNFCTKCKEGLYLHKGRCYPACPE
GSSAANGTMECSSPGGGGSGGGGSLPETGG
FcF2 sequence-with leader sequence (SEQ ID NO: 2)
METDTLLLWVLLLWVPGSTGEPKSCDKTHTCPPCPAPELLGGPSVFLFPPKPKDTLMISR
TPEVTCVVVDVSHEDPEVKFNWYVDGVEVHNAKTKPREEQYNSTYRVVSVLTVDHHD
WLNGKEYKCKVSNKALPAPIEKTISKAKGQPREPQVYTLPPSRDELTKNQVSLTCLVKG
FYPSDIAVEWESNGQPENNYKTTPPVLDSDGSFFLYSKLTVDKSRWQQGNVFSCSVMHE
ALHSHYTQKSLSLSPGKGGGGSGGGGSSRGIKGKRQRRISAEGSQACAKGCELCSEVNG
CLKCSPKLFILLERNDIRQVGVCLPSCPPGYFDARNPDMNKCIKCKIEHCEACFSHNFCT
KCKEGLYLHKGRCYPACPEGSSAANGTMECSSPGGGGSGGGGSLPETGG
FcF2 sequence-with 8x His (SEQ ID NO: 3)
EPKSCDKTHTCPPCPAPELLGGPSVFLFPPKPKDTLMISRTPEVTCVVVDVSHEDPEVKF
NWYVDGVEVHNAKTKPREEQYNSTYRVVSVLTVDHHDWLNGKEYKCKVSNKALPAPI
EKTISKAKGQPREPQVYTLPPSRDELTKNQVSLTCLVKGFYPSDIAVEWESNGQPENNY
KTTPPVLDSDGSFFLYSKLTVDKSRWQQGNVFSCSVMHEALHSHYTQKSLSLSPGKGG
GGSGGGGSSRGIKGKRQRRISAEGSQACAKGCELCSEVNGCLKCSPKLFILLERNDIRQV
GVCLPSCPPGYFDARNPDMNKCIKCKIEHCEACFSHNFCTKCKEGLYLHKGRCYPACPE
GSSAANGTMECSSPGGGGSGGGGSLPETGGHHHHHHHH
Fu1-Fu2 (FuFu) sequence (SEQ ID NO: 4)
EPKSCDKTHTCPPCPAPELLGGPSVFLFPPKPKDTLMISRTPEVTCVVVDVSHEDPEVKF
NWYVDGVEVHNAKTKPREEQYNSTYRVVSVLTVDHHDWLNGKEYKCKVSNKALPAPI
EKTISKAKGQPREPQVYTLPPSRDELTKNQVSLTCLVKGFYPSDIAVEWESNGQPENNY
KTTPPVLDSDGSFFLYSKLTVDKSRWQQGNVFSCSVMHEALHSHYTQKSLSLSPGK
(G₄S)₂ linker (SEQ ID NO: 5)
GGGGSGGGGS
LPETGG Sortase recognition site (SEQ ID NO: 6)
LPETGG
8xHis sequence (SEQ ID NO: 7)
HHHHHHHH
IgGk leader sequence (SEQ ID NO: 8)
METDTLLLWVLLLWVPGSTG
>Wild type IgG1-Fc domain: (SEQ ID NO: 9)
EPKSCDKTHTCPPCPAPELLGGPSVFLFPPKPKDTLMISRTPEVTCVVVDVSHEDPEVKF
NWYVDGVEVHNAKTKPREEQYNSTYRVVSVLTVLHQDWLNGKEYKCKVSNKALPAPI
EKTISKAKGQPREPQVYTLPPSRDELTKNQVSLTCLVKGFYPSDIAVEWESNGQPENNY
KTTPPVLDSDGSFFLYSKLTVDKSRWQQGNVFSCSVMHEALHNHYTQKSLSLSPGK
>Wild type IgG2-Fc domain (SEQ ID NO: 10)
ERKCCVECPPCPAPPVAGPSVFLFPPKPKDTLMISRTPEVTCVVVDVSHEDPEVQFNWY
VDGVEVHNAKTKPREEQFNSTFRVVSVLTVVHQDWLNGKEYKCKVSNKGLPAPIEKTI
SKTKGQPREPQVYTLPPSREEMTKNQVSLTCLVKGFYPSDISVEWESNGQPENNYKTTP
PMLDSDGSFFLYSKLTVDKSRWQQGNVFSCSVMHEALHNHYTQKSLSLSPGK
>Wild type IgG3-Fc domain (SEQ ID NO: 11)
ELKTPLGDTTHTCPRCPEPKSCDTPPPCPRCPEPKSCDTPPPCPRCPEPKSCDTPPPCPRCP
APELLGGPSVFLFPPKPKDTLMISRTPEVTCVVVDVSHEDPEVQFKWYVDGVEVHNAKT
KPREEQYNSTFRVVSVLTVLHQDWLNGKEYKCKVSNKALPAPIEKTISKTKGQPREPQV
YTLPPSREEMTKNQVSLTCLVKGFYPSDIAVEWESSGQPENNYNTTPPMLDSDGSFFLYS
KLTVDKSRWQQGNIFSCSVMHEALHNHFTQKSLSLSPGK
>Wild type IgG4-Fc domain (SEQ ID NO: 12)
ESKYGPPCPSCPAPEFLGGPSVFLFPPKPKDTLMISRTPEVTCVVVDVSQEDPEVQFNWY
VDGVEVHNAKTKPREEQFNSTYRVVSVLTVLHQDWLNGKEYKCKVSNKGLPSSIEKTIS
KAKGQPREPQVYTLPPSQEEMTKNQVSLTCLVKGFYPSDIAVEWESNGQPENNYKTTPP
VLDSDGSFFLYSRLTVDKSRWQEGNVFSCSVMHEALHNHYTQKSLSLSLGK
>DHS-IgG1 (SEQ ID NO: 13)
EPKSCDKTHTCPPCPAPELLGGPSVFLFPPKPKDTLMISRTPEVTCVVVDVSHEDPEVKF
NWYVDGVEVHNAKTKPREEQYNSTYRVVSVLTVDHHDWLNGKEYKCKVSNKALPAPI
EKTISKAKGQPREPQVYTLPPSRDELTKNQVSLTCLVKGFYPSDIAVEWESNGQPENNY
KTTPPVLDSDGSFFLYSKLTVDKSRWQQGNVFSCSVMHEALHSHYTQKSLSLSPGK
>DHS-IgG2 (SEQ ID NO: 14)
ERKCCVECPPCPAPPVAGPSVFLFPPKPKDTLMISRTPEVTCVVVDVSHEDPEVQFNWY
VDGVEVHNAKTKPREEQFNSTFRVVSVLTVDHHDWLNGKEYKCKVSNKGLPAPIEKTI
SKTKGQPREPQVYTLPPSREEMTKNQVSLTCLVKGFYPSDISVEWESNGQPENNYKTTP
PMLDSDGSFFLYSKLTVDKSRWQQGNVFSCSVMHEALHSHYTQKSLSLSPGK
>DHS-IgG3 (SEQ ID NO: 15)
ELKTPLGDTTHTCPRCPEPKSCDTPPPCPRCPEPKSCDTPPPCPRCPEPKSCDTPPPCPRCP
APELLGGPSVFLFPPKPKDTLMISRTPEVTCVVVDVSHEDPEVQFKWYVDGVEVHNAKT
KPREEQYNSTFRVVSVLTVDHHDWLNGKEYKCKVSNKALPAPIEKTISKTKGQPREPQV
YTLPPSREEMTKNQVSLTCLVKGFYPSDIAVEWESSGQPENNYNTTPPMLDSDGSFFLYS
KLTVDKSRWQQGNIFSCSVMHEALHSHFTQKSLSLSPGK
>DHS-IgG4 (SEQ ID NO: 16)
ESKYGPPCPSCPAPEFLGGPSVFLFPPKPKDTLMISRTPEVTCVVVDVSQEDPEVQFNWY
VDGVEVHNAKTKPREEQFNSTYRVVSVLTVDHHDWLNGKEYKCKVSNKGLPSSIEKTI
SKAKGQPREPQVYTLPPSQEEMTKNQVSLTCLVKGFYPSDIAVEWESNGQPENNYKTTP
PVLDSDGSFFLYSRLTVDKSRWQEGNVFSCSVMHEALHSHYTQKSLSLSLGK
FuFu (N137A mutant) amino acid (SEQ ID NO: 17)
METDTLLLWVLLLWVPGSTGEPKSCDKTHTCPPCPAPELLGGPSVFLFPPKPKDTLMISR
TPEVTCVVVDVSHEDPEVKFNWYVDGVEVHNAKTKPREEQYNSTYRVVSVLTVDHHD
WLNGKEYKCKVSNKALPAPIEKTISKAKGQPREPQVYTLPPSRDELTKNQVSLTCLVKG
FYPSDIAVEWESNGQPENNYKTTPPVLDSDGSFFLYSKLTVDKSRWQQGNVFSCSVMHE
ALHSHYTQKSLSLSPGKGGGGSGGGGSSRGIKGKRQRRISAEGSQACAKGCELCSEVNG
CLKCSPKLFILLERNDIRQVGVCLPSCPPGYFDARNPDMNKCIKCKIEHCEACFSHNFCT
KCKEGLYLHKGRCYPACPEGSSAAAGTMECSSPGGGGSGGGGSLPETGGHHHHHHHH
G₄S linker (SEQ ID NO: 18)
GGGGS
FcF2 sequence-with leader and 8x His (SEQ ID NO: 19)
METDTLLLWVLLLWVPGSTGEPKSCDKTHTCPPCPAPELLGGPSVFLFPPKPKDTLMISR
TPEVTCVVVDVSHEDPEVKFNWYVDGVEVHNAKTKPREEQYNSTYRVVSVLTVDHHD
WLNGKEYKCKVSNKALPAPIEKTISKAKGQPREPQVYTLPPSRDELTKNQVSLTCLVKG
FYPSDIAVEWESNGQPENNYKTTPPVLDSDGSFFLYSKLTVDKSRWQQGNVFSCSVMHE
ALHSHYTQKSLSLSPGKGGGGSGGGGSSRGIKGKRQRRISAEGSQACAKGCELCSEVNG
CLKCSPKLFILLERNDIRQVGVCLPSCPPGYFDARNPDMNKCIKCKIEHCEACFSHNFCT
KCKEGLYLHKGRCYPACPEGSSAANGTMECSSPGGGGSGGGGSLPETGGHHHHHHHH
DHSFc-(FuFu)₂ sequence (SEQ ID NO: 20)
EPKSCDKTHTCPPCPAPELLGGPSVFLFPPKPKDTLMISRTPEVTCVVVDVSHEDPEVKF
NWYVDGVEVHNAKTKPREEQYNSTYRVVSVLTVDHHDWLNGKEYKCKVSNKALPAPI
EKTISKAKGQPREPQVYTLPPSRDELTKNQVSLTCLVKGFYPSDIAVEWESNGQPENNY
KTTPPVLDSDGSFFLYSKLTVDKSRWQQGNVFSCSVMHEALHSHYTQKSLSLSPGKGG
GGSGGGGSSRGIKGKRQRRISAEGSQACAKGCELCSEVNGCLKCSPKLFILLERNDIRQV
GVCLPSCPPGYFDARNPDMNKCIKCKIEHCEACFSHNFCTKCKEGLYLHKGRCYPACPE
GSSAANGTMECSSPGGGGSGGGGSSRGIKGKRQRRISAEGSQACAKGCELCSEVNGCLK
CSPKLFILLERNDIRQVGVCLPSCPPGYFDARNPDMNKCIKCKIEHCEACFSHNFCTKCK
EGLYLHKGRCYPACPEGSSAANGTMECSSP
YVDEVDIIGGHEAK (SEQ ID NO: 21);
RVRRIIGGHEAK (SEQ ID NO: 22);
RVRRIIGGHEAK (SEQ ID NO: 23);
(I/A)(E/D)GRIIGGHEAK (SEQ ID NO: 24);
YEVDIIGGHEAK (SEQ ID NO: 25);
WEHDIIGGHEAK (SEQ ID NO: 26);
DVADIIGGHEAK (SEQ ID NO: 27);
DEHDIIGGHEAK (SEQ ID NO: 28);
DEVDIIGGHEAK (SEQ ID NO: 29);
DMQDIIGGHEAK (SEQ ID NO: 30);
LEVDIIGGHEAK (SEQ ID NO: 31);
LEHDIIGGHEAK (SEQ ID NO: 32);
VEIDIIGGHEAK (SEQ ID NO: 33);
VEHDIIGGHEAK (SEQ ID NO: 34);
IETDIIGGHEAK (SEQ ID NO: 35);
LETDIIGGHEAK (SEQ ID NO: 36)
IEADIIGGHEAK (SEQ ID NO: 37)
EDHS: (SEQ ID NO: 38)
EPKSCDKTHTCPPCPAPELLGGPSVFLFPPKPKDTLMISRTPEVTCVVEDVSHEDPEVKFN
WYVDGVEVHNAKTKPREEQYNSTYRVVSVLTVDHHDWLNGKEYKCKVSNKALPAPIE
KTISKAKGQPREPQVYTLPPSRDELTKNQVSLTCLVKGFYPSDIAVEWESNGQPENNYK
TTPPVLDSDGSFFLYSKLTVDKSRWQQGNVFSCSVMHEALHSHYTQKSLSLSPGK
EDHY: (SEQ ID NO: 39)
EPKSCDKTHTCPPCPAPELLGGPSVFLFPPKPKDTLMISRTPEVTCVVEDVSHEDPEVKFN
WYVDGVEVHNAKTKPREEQYNSTYRVVSVLTVDHHDWLNGKEYKCKVSNKALPAPIE
KTISKAKGQPREPQVYTLPPSRDELTKNQVSLTCLVKGFYPSDIAVEWESNGQPENNYK
TTPPVLDSDGSFFLYSKLTVDKSRWQQGNVFSCSVMHEALHYHYTQKSLSLSPGK
DHY (SEQ ID NO: 40):
EPKSCDKTHTCPPCPAPELLGGPSVFLFPPKPKDTLMISRTPEVTCVVVDVSHEDPEVKF
NWYVDGVEVHNAKTKPREEQYNSTYRVVSVLTVDHHDWLNGKEYKCKVSNKALPAPI
EKTISKAKGQPREPQVYTLPPSRDELTKNQVSLTCLVKGFYPSDIAVEWESNGQPENNY
KTTPPVLDSDGSFFLYSKLTVDKSRWQQGNVFSCSVMHEALHYHYTQKSLSLSPGK
LPXT (SEQ ID NO: 41)
LPET (SEQ ID NO: 42)
SPXT (SEQ ID NO: 104)
LAXT (SEQ ID NO: 43)
LSXT (SEQ ID NO: 44)
NPXT (SEQ ID NO: 45)
VPXT (SEQ ID NO: 46)
IPXT (SEQ ID NO: 47)
YPXR (SEQ ID NO: 48)
LPKTG (SEQ ID NO: 49)
LPATG (SEQ ID NO: 50)
LPNTG (SEQ ID NO: 51)
LPETG (SEQ ID NO: 52)
LPXAG (SEQ ID NO: 53)
LPNAG (SEQ ID NO: 54)
LPXTA (SEQ ID NO: 55)
LPNTA (SEQ ID NO: 56)
LGXTG (SEQ ID NO: 57)
LGATG (SEQ ID NO: 58)
IPXTG (SEQ ID NO: 59)
IPNTG (SEQ ID NO: 60)
IPETG (SEQ ID NO: 61)
LPKTGG (SEQ ID NO: 62)
LPATGG (SEQ ID NO: 63)
LPNTGG (SEQ ID NO: 64)
LPETGG (SEQ ID NO: 65)
LPXAGG (SEQ ID NO: 66)
LPNAGG (SEQ ID NO: 67)
LPXTAG (SEQ ID NO: 68)
LPNTAG (SEQ ID NO: 69)
LGXTGG (SEQ ID NO: 70)
LGATGG (SEQ ID NO: 71)
IPXTGG (SEQ ID NO: 72)
IPNTGG (SEQ ID NO: 73)
IPETGG (SEQ ID NO: 74)
GGGS (SEQ ID NO: 75)
FcF2 sequence-after sortase attachment (SEQ ID NO: 76)
EPKSCDKTHTCPPCPAPELLGGPSVFLFPPKPKDTLMISRTPEVTCVVVDVSHEDPEVKF
NWYVDGVEVHNAKTKPREEQYNSTYRVVSVLTVDHHDWLNGKEYKCKVSNKALPAPI
EKTISKAKGQPREPQVYTLPPSRDELTKNQVSLTCLVKGFYPSDIAVEWESNGQPENNY
KTTPPVLDSDGSFFLYSKLTVDKSRWQQGNVFSCSVMHEALHSHYTQKSLSLSPGKGG
GGSGGGGSSRGIKGKRQRRISAEGSQACAKGCELCSEVNGCLKCSPKLFILLERNDIRQV
GVCLPSCPPGYFDARNPDMNKCIKCKIEHCEACFSHNFCTKCKEGLYLHKGRCYPACPE
GSSAANGTMECSSPGGGGSGGGGSLPETGGG
FcF2 sequence-after sortase attachment including cleavable bond (SEQ
ID NO: 77)
EPKSCDKTHTCPPCPAPELLGGPSVFLFPPKPKDTLMISRTPEVTCVVVDVSHEDPEVKF
NWYVDGVEVHNAKTKPREEQYNSTYRVVSVLTVDHHDWLNGKEYKCKVSNKALPAPI
EKTISKAKGQPREPQVYTLPPSRDELTKNQVSLTCLVKGFYPSDIAVEWESNGQPENNY
KTTPPVLDSDGSFFLYSKLTVDKSRWQQGNVFSCSVMHEALHSHYTQKSLSLSPGKGG
GGSGGGGSSRGIKGKRQRRISAEGSQACAKGCELCSEVNGCLKCSPKLFILLERNDIRQV
GVCLPSCPPGYFDARNPDMNKCIKCKIEHCEACFSHNFCTKCKEGLYLHKGRCYPACPE
GSSAANGTMECSSPGGGGSGGGGSLPETGGGV-citrulline
RSPO2-Fu1 (33-84)-(SEQ ID NO: 78)
SYVSNPICKGCLSCSKDNGCSRCQQKLFFFLRREGMRQYGECLHSCPSGYYG
RSPO2-Fu2 (90-134)-(SEQ ID NO: 79)
MNRCARCRIENCDSCFSKDFCTKCKVGFYLHRGRCFDECPDGFAP
RSPO3-Fu1 (35-86)-(SEQ ID NO: 80)
PNVSQGCQGGCATCSDYNGCLSCKPRLFFALERIGMKQIGVCLSSCPSGYYG
RSPO3-Fu2 (92-135)-(SEQ ID NO: 81)
INKCTKCKADCDTCFNKNFCTKCKSGFYLHLGKCLDNCPEGLEA
RSPO4-Fu1 (28-81)-(SEQ ID NO: 82)
GTGLGGNCTGCIICSEENGCSTCQQRLFLFIRREGIRQYGKCLHDCPPGYFGIR
RSPO4-Fu2 (85-128)-(SEQ ID NO: 83)
VNRCKKCGATCESCFSQDFCIRCKRQFYLYKGKCLPTCPPGTLA
(EAAAK)₃ (SEQ ID NO: 84)
EAAAKEAAAKEAAAK
RSPO2-Fu1-Fu2: (SEQ ID NO: 85)
SYVSNPICKGCLSCSKDNGCSRCQQKLFFFLRREGMRQYGECLHSCPSGYYGHRAPDM
NRCARCRIENCDSCFSKDFCTKCKVGFYLHRGRCFDECPDGFAP
RSPO3-Fu1-Fu2: (SEQ ID NO: 86)
PNVSQGCQGGCATCSDYNGCLSCKPRLFFALERIGMKQIGVCLSSCPSGYYGTRYPDIN
KCTKCKADCDTCFNKNFCTKCKSGFYLHLGKCLDNCPEGLEA
RSPO4-Fu1-Fu2: (SEQ ID NO: 87)
GTGLGGNCTGCIICSEENGCSTCQQRLFLFIRREGIRQYGKCLHDCPPGYFGIRGQEVNRC
KKCGATCESCFSQDFCIRCKRQFYLYKGKCLPTCPPGTLA
RSPO1-Fu1: (SEQ ID NO: 88)
AEGSQACAKGCELCSEVNGCLKCSPKLFILLERNDIRQVGVCLPSCPPGYFD
RSPO1-Fu2: (SEQ ID NO: 89)
MNKCIKCKIEHCEACFSHNFCTKCKEGLYLHKGRCYPACPEGSSA
FcF2 (R28A) mutant-nucleotide sequence (SEQ ID NO: 97)
GCTAGCGCCACCATGGAAACCGATACACTGCTGCTGTGGGTGCTGCTCCTTTGGGTG
CCCGGATCTACAGGCGAGCCTAAGAGCTGCGACAAGACCCACACCTGTCCTCCATG
TCCTGCTCCAGAACTGCTCGGCGGACCTTCCGTGTTCCTGTTTCCTCCAAAGCCTAAG
GACACCCTGATGATCAGCAGAACCCCTGAAGTGACCTGCGTGGTGGTGGATGTGTCC
CACGAGGATCCCGAAGTGAAGTTCAATTGGTACGTGGACGGCGTGGAAGTGCACAA
CGCCAAGACCAAGCCTAGAGAGGAACAGTACAACAGCACCTACAGAGTGGTGTCCG
TGCTGACCGTGGACCACCACGATTGGCTGAACGGCAAAGAGTACAAGTGCAAGGTG
TCCAACAAGGCCCTGCCTGCTCCTATCGAGAAAACCATCAGCAAGGCCAAGGGCCA
GCCTAGGGAACCCCAGGTTTACACACTGCCTCCAAGCAGGGACGAGCTGACCAAGA
ATCAGGTGTCCCTGACCTGCCTGGTCAAGGGCTTCTACCCTTCCGATATCGCCGTGG
AATGGGAGAGCAATGGCCAGCCTGAGAACAACTACAAGACAACCCCTCCTGTGCTG
GACAGCGACGGCTCATTCTTCCTGTACAGCAAGCTGACAGTGGACAAGAGCAGATG
GCAGCAGGGCAACGTGTTCAGCTGCAGCGTGATGCACGAAGCCCTGCACAGCCACT
ACACCCAGAAGTCCCTGTCTCTGAGCCCTGGAAAAGGCGGCGGAGGATCTGGCGGA
GGCGGATCTTCTAGAGGCATCAAGGGCAAGGCCCAGCGGAGAATCTCTGCCGAGGG
ATCTCAGGCCTGTGCCAAGGGCTGTGAACTGTGCAGCGAAGTGAACGGCTGCCTGA
AGTGCAGCCCCAAGCTGTTCATCCTGCTGGAACGGAACGACATCCGGCAAGTGGGA
GTGTGCCTGCCTTCTTGTCCTCCTGGCTACTTCGACGCCAGAAATCCCGACATGAAC
AAGTGCATTAAGTGCAAGATCGAGCACTGCGAGGCCTGCTTCAGCCACAACTTCTGC
ACCAAGTGCAAAGAGGGCCTGTACCTGCACAAGGGCAGATGCTATCCTGCCTGTCC
TGAGGGAAGCTCTGCCGCCAATGGCACAATGGAATGTAGCTCTCCAGGTGGCGGAG
GAAGCGGTGGCGGCGGATCTCTTCCAGAAACAGGCGGACACCATCACCATCATCAC
CACCACTGATGACTCGAG
FcF2 (R28A) mutant-amino acid sequence (SEQ ID NO: 98)
ASATMETDTLLLWVLLLWVPGSTGEPKSCDKTHTCPPCPAPELLGGPSVFLFPPKPKDTL
MISRTPEVTCVVVDVSHEDPEVKFNWYVDGVEVHNAKTKPREEQYNSTYRVVSVLTV
DHHDWLNGKEYKCKVSNKALPAPIEKTISKAKGQPREPQVYTLPPSRDELTKNQVSLTC
LVKGFYPSDIAVEWESNGQPENNYKTTPPVLDSDGSFFLYSKLTVDKSRWQQGNVFSCS
VMHEALHSHYTQKSLSLSPGKGGGGSGGGGSSRGIKGKAQRRISAEGSQACAKGCELCS
EVNGCLKCSPKLFILLERNDIRQVGVCLPSCPPGYFDARNPDMNKCIKCKIEHCEACFSH
NFCTKCKEGLYLHKGRCYPACPEGSSAANGTMECSSPGGGGSGGGGSLPETGGHHHHH
HHH--
Fu1-Fu2 (R28A) mutant-amino acid sequence (SEQ ID NO: 90)
SRGIKGKAQRRISAEGSQACAKGCELCSEVNGCLKCSPKLFILLERNDIRQVGVCLPSCPP
GYFDARNPDMNKCIKCKIEHCEACFSHNFCTKCKEGLYLHKGRCYPACPEGSSAANGT
MECSSP
FcF2 (R30A) mutant-nucleotide sequence (SEQ ID NO: 101)
GCTAGCGCCACCATGGAAACCGATACACTGCTGCTGTGGGTGCTGCTCCTTTGGGTG
CCCGGATCTACAGGCGAGCCTAAGAGCTGCGACAAGACCCACACCTGTCCTCCATG
TCCTGCTCCAGAACTGCTCGGCGGACCTTCCGTGTTCCTGTTTCCTCCAAAGCCTAAG
GACACCCTGATGATCAGCAGAACCCCTGAAGTGACCTGCGTGGTGGTGGATGTGTCC
CACGAGGATCCCGAAGTGAAGTTCAATTGGTACGTGGACGGCGTGGAAGTGCACAA
CGCCAAGACCAAGCCTAGAGAGGAACAGTACAACAGCACCTACAGAGTGGTGTCCG
TGCTGACCGTGGACCACCACGATTGGCTGAACGGCAAAGAGTACAAGTGCAAGGTG
TCCAACAAGGCCCTGCCTGCTCCTATCGAGAAAACCATCAGCAAGGCCAAGGGCCA
GCCTAGGGAACCCCAGGTTTACACACTGCCTCCAAGCAGGGACGAGCTGACCAAGA
ATCAGGTGTCCCTGACCTGCCTGGTCAAGGGCTTCTACCCTTCCGATATCGCCGTGG
AATGGGAGAGCAATGGCCAGCCTGAGAACAACTACAAGACAACCCCTCCTGTGCTG
GACAGCGACGGCTCATTCTTCCTGTACAGCAAGCTGACAGTGGACAAGAGCAGATG
GCAGCAGGGCAACGTGTTCAGCTGCAGCGTGATGCACGAAGCCCTGCACAGCCACT
ACACCCAGAAGTCCCTGTCTCTGAGCCCTGGAAAAGGCGGCGGAGGATCTGGCGGA
GGCGGATCTTCTAGAGGCATCAAGGGCAAGAGACAGGCCAGAATCTCTGCCGAGGG
ATCTCAGGCCTGTGCCAAGGGCTGTGAACTGTGCAGCGAAGTGAACGGCTGCCTGA
AGTGCAGCCCCAAGCTGTTCATCCTGCTGGAACGGAACGACATCCGGCAAGTGGGA
GTGTGCCTGCCTTCTTGTCCTCCTGGCTACTTCGACGCCAGAAATCCCGACATGAAC
AAGTGCATTAAGTGCAAGATCGAGCACTGCGAGGCCTGCTTCAGCCACAACTTCTGC
ACCAAGTGCAAAGAGGGCCTGTACCTGCACAAGGGCAGATGCTATCCTGCCTGTCC
TGAGGGAAGCTCTGCCGCCAATGGCACAATGGAATGTAGCTCTCCAGGTGGCGGAG
GAAGCGGTGGCGGCGGATCTCTTCCAGAAACAGGCGGACACCATCACCATCATCAC
CACCACTGATGACTCGAG
FcF2 (R30A) mutant-amino acid sequence (SEQ ID NO: 102)
ASATMETDTLLLWVLLLWVPGSTGEPKSCDKTHTCPPCPAPELLGGPSVFLFPPKPKDTL
MISRTPEVTCVVVDVSHEDPEVKFNWYVDGVEVHNAKTKPREEQYNSTYRVVSVLTV
DHHDWLNGKEYKCKVSNKALPAPIEKTISKAKGQPREPQVYTLPPSRDELTKNQVSLTC
LVKGFYPSDIAVEWESNGQPENNYKTTPPVLDSDGSFFLYSKLTVDKSRWQQGNVFSCS
VMHEALHSHYTQKSLSLSPGKGGGGSGGGGSSRGIKGKRQARISAEGSQACAKGCELCS
EVNGCLKCSPKLFILLERNDIRQVGVCLPSCPPGYFDARNPDMNKCIKCKIEHCEACFSH
NFCTKCKEGLYLHKGRCYPACPEGSSAANGTMECSSPGGGGSGGGGSLPETGGHHHHH
HHH--LE
Fu1-Fu2 (R30A) mutant-nucleotide sequence (SEQ ID NO: 100)
TCTAGAGGCATCAAGGGCAAGAGACAGGCCAGAATCTCTGCCGAGGGATCTCAGGC
CTGTGCCAAGGGCTGTGAACTGTGCAGCGAAGTGAACGGCTGCCTGAAGTGCAGCC
CCAAGCTGTTCATCCTGCTGGAACGGAACGACATCCGGCAAGTGGGAGTGTGCCTG
CCTTCTTGTCCTCCTGGCTACTTCGACGCCAGAAATCCCGACATGAACAAGTGCATT
AAGTGCAAGATCGAGCACTGCGAGGCCTGCTTCAGCCACAACTTCTGCACCAAGTG
CAAAGAGGGCCTGTACCTGCACAAGGGCAGATGCTATCCTGCCTGTCCTGAGGGAA
GCTCTGCCGCCAATGGCACAATGGAATGTAGCTCTCCA
Fu1-Fu2 (R22-R31 deletion) (SEQ ID NO: 92)
SISAEGSQACAKGCELCSEVNGCLKCSPKLFILLERNDIRQVGVCLPSCPPGYFDARNPD
MNKCIKCKIEHCEACFSHNFCTKCKEGLYLHKGRCYPACPEGSSAANGTMECSSP
Fu1-Fu2 (K25-R31 deletion) (SEQ ID NO: 93)
SRGIISAEGSQACAKGCELCSEVNGCLKCSPKLFILLERNDIRQVGVCLPSCPPGYFDARN
PDMNKCIKCKIEHCEACFSHNFCTKCKEGLYLHKGRCYPACPEGSSAANGTMECSSP
Fu1-Fu2 (R28-R31 deletion) (SEQ ID NO: 94)
SRGIKGKISAEGSQACAKGCELCSEVNGCLKCSPKLFILLERNDIRQVGVCLPSCPPGYFD
ARNPDMNKCIKCKIEHCEACFSHNFCTKCKEGLYLHKGRCYPACPEGSSAANGTMECSS
P
Fu1-Fu2 (R22-K27 deletion) (SEQ ID NO: 95)
SRQRRISAEGSQACAKGCELCSEVNGCLKCSPKLFILLERNDIRQVGVCLPSCPPGYFDA
RNPDMNKCIKCKIEHCEACFSHNFCTKCKEGLYLHKGRCYPACPEGSSAANGTMECSSP
Fu1-Fu2 (S21-Q38 deletion) (SEQ ID NO: 96)
ACAKGCELCSEVNGCLKCSPKLFILLERNDIRQVGVCLPSCPPGYFDARNPDMNKCIKC
KIEHCEACFSHNFCTKCKEGLYLHKGRCYPACPEGSSAANGTMECSSP
Amino acid sequence of FcST4 (SEQ ID NO: 105)
METDTLLLWVLLLWVPGSTGEPKSCDKTHTCPPCPAPELLGGPSVFLFPPKPKDTLMISR
TPEVTCVVVDVSHEDPEVKFNWYVDGVEVHNAKTKPREEQYNSTYRVVSVLTVDHHD
WLNGKEYKCKVSNKALPAPIEKTISKAKGQPREPQVYTLPPSRDELTKNQVSLTCLVKG
FYPSDIAVEWESNGQPENNYKTTPPVLDSDGSFFLYSKLTVDKSRWQQGNVFSCSVMHE
ALHSHYTQKSLSLSPGKGGGGSGGGGSSRGIKGKRQRRISAEGSQACAKGCELCSEVNG
CLKCSPKLFILLERNDIRQVGVCLPSCPPGYFDARNPDMNKCIKCKIEHCEACFSHNFCT
KCKEGLYLHKGRCYPACPEGSSAANGTMECSSPGGGGSGGGGSSRGIKGKRQRRISAEG
SQACAKGCELCSEVNGCLKCSPKLFILLERNDIRQVGVCLPSCPPGYFDARNPDMNKCIK
CKIEHCEACFSHNFCTKCKEGLYLHKGRCYPACPEGSSAANGTMECSSPGGGGSGGGGS
LPETGGHHHHHHHH
Nucleotide sequence of FcST4 (SEQ ID NO: 103)
AGCAGCGCTAGCGCCACCATGGAAACCGATACACTGCTGCTGTGGGTGCTGCTCCTT
TGGGTGCCCGGATCTACAGGCGAGCCTAAGAGCTGCGACAAGACCCACACCTGTCC
TCCATGTCCTGCTCCAGAACTGCTCGGCGGACCTTCCGTGTTCCTGTTTCCTCCAAAG
CCTAAGGACACCCTGATGATCAGCAGAACCCCTGAAGTGACCTGCGTGGTGGTGGA
TGTGTCCCACGAGGATCCCGAAGTGAAGTTCAATTGGTACGTGGACGGCGTGGAAG
TGCACAACGCCAAGACCAAGCCTAGAGAGGAACAGTACAACAGCACCTACAGAGT
GGTGTCCGTGCTGACCGTGGACCACCACGATTGGCTGAACGGCAAAGAGTACAAGT
GCAAGGTGTCCAACAAGGCCCTGCCTGCTCCTATCGAGAAAACCATCAGCAAGGCC
AAGGGCCAGCCTAGGGAACCCCAGGTTTACACACTGCCTCCAAGCAGGGACGAGCT
GACCAAGAATCAGGTGTCCCTGACCTGCCTGGTCAAGGGCTTCTACCCTTCCGATAT
CGCCGTGGAATGGGAGAGCAATGGCCAGCCTGAGAACAACTACAAGACAACCCCTC
CTGTGCTGGACAGCGACGGCTCATTCTTCCTGTACAGCAAGCTGACAGTGGACAAGA
GCAGATGGCAGCAGGGCAACGTGTTCAGCTGCAGCGTGATGCACGAAGCCCTGCAC
AGCCACTACACCCAGAAGTCCCTGTCTCTGAGCCCTGGAAAAGGCGGCGGAGGATC
TGGCGGAGGCGGATCTTCTAGAGGCATCAAGGGCAAGAGACAGCGGAGAATCTCTG
CCGAGGGATCTCAGGCCTGTGCCAAGGGCTGTGAACTGTGCAGCGAAGTGAACGGC
TGCCTGAAGTGCAGCCCCAAGCTGTTCATCCTGCTGGAACGGAACGACATCCGGCA
AGTGGGAGTGTGCCTGCCTTCTTGTCCTCCTGGCTACTTCGACGCCAGAAATCCCGA
CATGAACAAGTGCATTAAGTGCAAGATCGAGCACTGCGAGGCCTGCTTCAGCCACA
ACTTCTGCACCAAGTGCAAAGAGGGCCTGTACCTGCACAAGGGCAGATGCTATCCT
GCCTGTCCTGAGGGAAGCTCTGCCGCCAATGGCACAATGGAATGTAGCTCTCCAGGT
GGCGGAGGAAGCGGTGGCGGCGGATCTCTTCCAGAAACAGGCGGACACCATCACCA
TCATCACCACCACTGATGACTCGAGAGCAGC

1. A compound comprising one or more cytotoxic agent(s) conjugated to a polypeptide comprising one or more LGR binding domain(s), wherein
(i) the polypeptide further comprises an Fc region, and/or
(ii) the polypeptide comprises at least two copies of the LGR binding domain; and
wherein each LGR binding domain comprises a polypeptide having at least 90%, more preferably at least 95% sequence identity to at least one of SEQ ID NOs: 4, 78-83, 85-89, 90-96, 98 or 102.

2. The compound of claim 1, wherein the LGR binding domain(s) each comprise an amino acid sequence independently selected from SEQ ID NO:4, SEQ ID NO:85, SEQ ID NO:86, or SEQ ID NO:87.

3. The compound of claim 1, wherein the LGR binding domain is from human R-spondin-1 (hR-spondin-1), human R-spondin-2 (hR-spondin-2), human R-spondin-3 (hR-spondin-3), or human R-spondin-4 (hR-spondin-4).

4. The compound of claim 1, wherein the LGR binding domain(s) comprise an amino acid sequence independently selected from FuFu (SEQ ID NO:4) or FuFu N137A (SEQ ID NO:17).

5. The compound of claim 1, wherein the LGR binding domain comprises a substitution mutation at position R28 mutation or R30 with numbering according to Kabat.

6. The compound of claim 5, wherein the substitution mutation is arginine to alanine.

7. The compound of claim 6, wherein the substitution mutation is R28A.

8. The compound of claim 7, wherein the LGR binding domain comprises Fu1-Fu2 (R30A) mutant (SEQ ID NO: 91) or the Fu1-Fu2 (R30A) mutant (SEQ ID NO: 100).

9. The compound of claim 7, wherein the LGR binding domain comprises Fu1-Fu2 (R30A) mutant (SEQ ID NO: 100).

10. The compound of claim 1, wherein the LGR binding domain comprises Fu1-Fu2 (R22-R31 deletion) (SEQ ID NO: 92), Fu1-Fu2 (K25-R31 deletion) (SEQ ID NO: 93), Fu1-Fu2 (R28-R31 deletion) (SEQ ID NO: 94), or Fu1-Fu2 (R22-K27 deletion) (SEQ ID NO: 95).

11. The compound of claim 1, wherein the polypeptide comprises FcST4 (SEQ ID NO: 105).

12. The compound of claim 1, wherein the Fc region is N-terminal relative to the LGR binding domain, and/or wherein the polypeptide comprises in an N-to-C direction: the Fc region and the LGR binding domain.

13. The compound of claim 1, wherein the Fc region is an IgG Fc domain.

14. The compound of claim 13, wherein the polypeptide comprises SEQ ID NO:9, SEQ ID NO:10, SEQ ID NO:11, or SEQ ID NO:12.

15. The compound of claim 1, wherein the polypeptide comprises SEQ ID NO:78, SEQ ID NO: 80, SEQ ID NO: 82, or SEQ ID NO:88; and wherein the polypeptide does not comprise SEQ ID NO: 79, SEQ ID NO:81, SEQ ID NO:83, or SEQ ID NO:89.

16. The compound of claim 1, wherein the polypeptide comprises SEQ ID NO: 79, SEQ ID NO:81, SEQ ID NO:83, or SEQ ID NO:89; and wherein the polypeptide does not comprise SEQ ID NO:78, SEQ ID NO: 80, SEQ ID NO: 82, or SEQ ID NO:88.

17. The compound of claim 12, wherein the human IgG Fc domain capable of binding human FcRn at an acidic pH, wherein the Fc domain has substitution mutations of:
(i) aspartic acid at position 309 (L/V309D);
(ii) histidine at position 311 (Q311H); and
(iii) a substitution mutation at position 434 of serine (N434S) or tyrosine (N434Y);
with amino acid position numbering being according to the Kabat system; wherein the Fc domain binds FcRn at an acidic pH with an affinity higher than the wild-type.

18. The compound of claim 17, wherein the substitution mutation at position 434 is serine (N434S).

19. The compound of claim 17, wherein the substitution mutation at position 434 is tyrosine (N434Y).

20. The compound of claim 17, wherein the polypeptide comprises SEQ ID NO:13, SEQ ID NO:14, SEQ ID NO:15, or SEQ ID NO:16.

21. The compound of claim 20, wherein the polypeptide comprises SEQ ID NO:13.

22. The compound of claim 17, wherein the Fc domain is glycosylated.

23-25. (canceled)

26. The compound of claim 25, wherein the Fc domain exhibits: (i) enhanced binding at pH 5.8 and (ii) reduced binding or no detectable binding at pH 7.4 for FcRn, as compared to the wild-type.

27. The compound of claim 17, wherein the Fc domain is aglycosylated.

28. The compound of claim 27, wherein the Fc domain has a substitution mutation of glutamic acid at position 264 (V264E).

29. The compound of claim 17, wherein the IgG is IgG1, IgG2, IgG3, or IgG4.

30. The compound of claim 17, wherein the IgG is IgG1.

31. The compound of claim 17, wherein the Fc domain comprises substitution mutations:
(i) IgG1-Fc EDHS (V264E; L309D; Q311H; N434S),
(ii) IgG1-Fc EDHY (V264E; L309D; Q311H; N434Y),
(iii) IgG1-Fc DHS (L309D; Q311H; N434S),
(iv) IgG1-Fc DHY (L309D; Q311H; N434Y),
(v) IgG2-DHS (V309D; Q311H; N434S),
(vi) IgG3-DHS (L309D; Q311H; N434S), or
(vii) IgG4-DHS (L309D; Q311H; N434S).

32. The compound of claim 31, wherein the Fc domain is IgG1-Fc DHS (L309D; Q311H; N434S).

33. The compound of claim 12, wherein the compound is dimerized via disulfide bonds formed in the Fc domain.

34. The compound of claim 12, wherein the Fc domain is separated from the LGR binding domain by a linker.

35. The compound of claim 34, wherein the linker comprises G₄S (SEQ ID NO:18) or (G₄S)₂ (SEQ ID NO:5).

36. The compound of claim 12, wherein the Fc domain is not separated from the LGR binding domain by a linker, or wherein the polypeptide does not comprise a linker.

37. The compound of claim 34, wherein the polypeptide comprises from the N-terminal to C-terminal ends: the Fc domain and the LGR binding domain; or wherein Fc domain is closer to the N-terminal of the polypeptide than the LGR binding domain.

38. The compound of claim 1, wherein the compound comprises two copies of FuFu (SEQ ID NO:4) or FuFu N137A (SEQ ID NO:17).

39. The compound of claim 38, wherein the two copies of FuFu (SEQ ID NO:4) or FuFu N137A (SEQ ID NO:17) are separated via a linker, preferably a G₄S linker (SEQ ID NO:18) or a (G₄S)₂ linker (SEQ ID NO:5).

40. The compound of claim 39, wherein the compound comprises SEQ ID NO: 1, SEQ ID NO:2, SEQ ID NO:3, or SEQ ID NO:19.

41. The compound of claim 1, wherein the compound comprises SEQ ID NO:1.

42. The compound of claim 1, wherein the compound comprises SEQ ID NO:20.

43. The compound of claim 1, wherein the polypeptide comprises a leader sequence.

44. The compound of claim 43, wherein the leader sequence is an endogenous leader sequence, an IgG leader sequence, or an IgK leader sequence.

45. The compound of claim 44, wherein the IgG leader sequence is IgGk leader sequence (SEQ ID NO: 8).

46. The compound of claim 1, wherein the polypeptide does not comprise a leader sequence.

47. The compound of claim 1, wherein the first cytotoxic agent is a conjugated drug.

48. The compound of claim 47, wherein the drug is a maytansinoid, auristatin, amanitin, calicheamycin, psymberin, duocarmycin, anthracyclin, camptothecin, doxorubicin, taxol, tubulysin, eribluin, or pyrrolobenzodiazepine.

49. The compound of claim 48, wherein the drug is an auristatin.

50. The compound of claim 49, wherein the auristatin is monomethyl auristatin E (MMAE), Monomethyl auristatin F (MMAF), or PF-06380101.

51. The compound of claim 49, wherein the auristatin is monomethyl auristatin E (MMAE).

52. The compound of claim 47, wherein the drug is a camptothecin analog.

53. The compound of claim 52, wherein the camptothecin analog is topotecan, irinotecan, belotecan, or deruxtecan.

54. The compound of claim 47, wherein the drug is an antracycline analog.

55. The compound of claim 54, wherein the antracycline analog is PNU-159682 (CAS No.: 202350-68-3).

56. The compound of claim 47, wherein the conjugated drug is attached to the polypeptide via a linker.

57. The compound of claim 56, wherein the linker is a protease-cleavable linker.

58. The compound of claim 57, wherein the protease-cleavable linker is citrulline-valine.

59. The compound of claim 1, wherein the compound comprises at least one spacer positioned between the cytotoxic agent and the LGR binding domain.

60. The compound of claim 59, wherein the compound comprises two linkers.

61. The compound of claim 59, wherein the linker comprises G₄S (SEQ ID NO:18) or (G₄S)₂ (SEQ ID NO:5).

62. The compound of claim 1, wherein the cytotoxic moiety is a cytotoxic protein.

63. The compound of claim 62, wherein the cytotoxic protein is a serine protease.

64. The compound of claim 63, wherein the serine protease is a granzyme B (GrB).

65. The compound of claim 1, wherein the compound comprises SEQ ID NO:1 covalently attached to monomethyl auristatin E (MMAE).

66. The compound of claim 1, wherein the compound comprises SEQ ID NO:2 covalently attached to monomethyl auristatin E (MMAE).

67. (canceled)

68. The compound of claim 1, wherein the cytotoxic agent has been covalently bound to the polypeptide via a sortase linker.

69. The compound of claim 68, wherein the sortase is a sortase A linker or a sortase E linker.

70. The compound of claim 1, wherein the compound comprises a sortase linker between the LGR binding domain and the cytotoxic agent.

71. The compound of claim 70, wherein the cytotoxic agent has been covalently bound to the polypeptide via a sortase.

72. The compound of claim 71, wherein the sortase is sortase A or sortase E.

73. The compound of claim 70, wherein the sortase linker comprises the sequence LPXT(G)ₙ or LAHTGG (SEQ ID NO: 106), wherein n=1-10.

74. The compound of claim 73, wherein the sortase linker is LPETGG (SEQ ID NO:6).

75. The compound of claim 1, wherein the compound further comprises a second cytotoxic agent.

76-77. (canceled)

78. The compound of claim 77, wherein the first cytotoxic agent is covalently attached to a first sortase linker on the N-terminal side of the polypeptide, and wherein the second cytotoxic agent is covalently attached to a second sortase linker on the N-terminal side of the polypeptide.

79. The compound of claim 78, wherein the first sortase linker comprises the sequence LPXT(G)n, wherein n=1-10.

80. The compound of claim 79, wherein the first sortase linker is LPETGG (SEQ ID NO:6).

81. The compound of claim 78, wherein the second sortase linker is LAHTGG (SEQ ID NO: 106).

82-93. (canceled)

94. The compound of claim 1, wherein the polypeptide comprises SEQ ID NO:76.

95. The compound of claim 94, wherein the polypeptide comprises SEQ ID NO:77.

96. The compound of claim 95, wherein the polypeptide is covalently attached to -PABA-MMAE.

97. The compound of claim 1, wherein the compound is comprised in a pharmaceutical composition.

98. A pharmaceutical composition comprising a compound of claim 1.

99. (canceled)

100. A nucleic acid encoding the polypeptide of claim 1.

101. A host cell comprising the nucleic acid of claim 100.

102-105. (canceled)

106. A method of producing a therapeutic compound that binds an LGR receptor, wherein the method comprises:
(a) expressing the polypeptide encoded by the nucleic acid of claim 100 in a cell, wherein the polypeptide comprises a sortase linker at a terminal end of the polypeptide;
(b) obtaining the polypeptide; and
(c) contacting a first cytotoxic agent and the polypeptide with a first transpeptidase, thereby covalently bonding the first cytotoxic compound to the polypeptide.

107-142. (canceled)

143. A method of manufacturing a polypeptide comprising:
(a) expressing a nucleic acid of claim 100 in a cell under conditions to produce the encoded polypeptide; and
(b) purifying the polypeptide from the cell.

144. A method of treating a subject with a cell proliferative disease comprising administering to the subject an effective amount of the compound of claim 1.

145-155. (canceled)

157. A method for inhibiting proliferation of cancer stem cells, the method comprising contacting said cancer stem cells with the compound of claim 1.

158. A method for treating cancer, the method comprising contacting said cancer stem cells with the compound of claim 1.

159. A method for reducing the spread of cancer cells and/or cancer stem cells, the method comprising contacting said cancer stem cells with the compound of claim 1.


SEQUENCE LISTING (106 sequences)

SEQ ID NO: 1 - protein, 383 aa, synthetic construct
EPKSCDKTHTCPPCPAPELLGGPSVFLFPPKPKDTLMISRTPEVTCVVVDVSHEDPEVKFNWYVDGVEVHNAKTKPREEQYNSTYRVVSVLTVDHHDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHSHYTQKSLSLSPGKGGGGSGGGGSSRGIKGKRQRRISAEGSQACAKGCELCSEVNGCLKCSPKLFILLERNDIRQVGVCLPSCPPGYFDARNPDMNKCIKCKIEHCEACFSHNFCTKCKEGLYLHKGRCYPACPEGSSAANGTMECSSPGGGGSGGGGSLPETGG

SEQ ID NO: 2 - protein, 403 aa, synthetic construct
METDTLLLWVLLLWVPGSTGEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVDHHDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHSHYTQKSLSLSPGKGGGGSGGGGSSRGIKGKRQRRISAEGSQACAKGCELCSEVNGCLKCSPKLFILLERNDIRQVGVCLPSCPPGYFDARNPDMNKCIKCKIEHCEACFSHNFCTKCKEGLYLHKGRCYPACPEGSSAANGTMECSSPGGGGSGGGGSLPETGG

SEQ ID NO: 3 - protein, 391 aa, synthetic construct
EPKSCDKTHTCPPCPAPELLGGPSVFLFPPKPKDTLMISRTPEVTCVVVDVSHEDPEVKFNWYVDGVEVHNAKTKPREEQYNSTYRVVSVLTVDHHDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHSHYTQKSLSLSPGKGGGGSGGGGSSRGIKGKRQRRISAEGSQACAKGCELCSEVNGCLKCSPKLFILLERNDIRQVGVCLPSCPPGYFDARNPDMNKCIKCKIEHCEACFSHNFCTKCKEGLYLHKGRCYPACPEGSSAANGTMECSSPGGGGSGGGGSLPETGGHHHHHHHH

SEQ ID NO: 4 - protein, 232 aa, synthetic construct
EPKSCDKTHTCPPCPAPELLGGPSVFLFPPKPKDTLMISRTPEVTCVVVDVSHEDPEVKFNWYVDGVEVHNAKTKPREEQYNSTYRVVSVLTVDHHDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHSHYTQKSLSLSPGK

SEQ ID NO: 5 - protein, 10 aa, synthetic construct
GGGGSGGGGS

SEQ ID NO: 6 - protein, 6 aa, synthetic construct
LPETGG

SEQ ID NO: 7 - protein, 8 aa, synthetic construct
HHHHHHHH

SEQ ID NO: 8 - protein, 20 aa, synthetic construct
METDTLLLWVLLLWVPGSTG

SEQ ID NO: 9 - protein, 232 aa, synthetic construct
EPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 10 - protein, 228 aa, synthetic construct
ERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDISVEWESNGQPENNYKTTPPMLDSDGSFFLYSKLTVDKSRWQQGNVFSCSVMHEALHNHYTQKSLSLSPGK

SEQ ID NO: 11 - protein, 279 aa, synthetic construct
ELKTPLGDTTHTCPRCPEPKSCDTPPPCPRCPEPKSCDTPPPCPRCPEPKSCDTPPPCPRCPAPELLGGPSVFLFPPKPKDTLMISRTPEVTCVVVDVSHEDPEVQFKWYVDGVEVHNAKTKPREEQYNSTFRVVSVLTVLHQDWLNGKEYKCKVSNKALPAPIEKTISKTKGQPREPQVYTLPPSREEMTKNQVSLTCLVKGFYPSDIAVEWESSGQPENNYNTTPPMLDSDGSFFLYSKLTVDKSRWQQGNIFSCSVMHEALHNHFTQKSLSLSPGK

SEQ ID NO: 12 - protein, 229 aa, synthetic construct
ESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 13 - protein, 232 aa, synthetic construct; identical to SEQ ID NO: 4

SEQ ID NO: 14 - protein, 228 aa, synthetic construct
ERKCCVECPPCPAPPVAGPSVFLFPPKPKDTLMISRTPEVTCVVVDVSHEDPEVQFNWYVDGVEVHNAKTKPREEQFNSTFRVVSVLTVDHHDWLNGKEYKCKVSNKGLPAPIEKTISKTKGQPREPQVYTLPPSREEMTKNQVSLTCLVKGFYPSDISVEWESNGQPENNYKTTPPMLDSDGSFFLYSKLTVDKSRWQQGNVFSCSVMHEALHSHYTQKSLSLSPGK

SEQ ID NO: 15 - protein, 279 aa, synthetic construct
ELKTPLGDTTHTCPRCPEPKSCDTPPPCPRCPEPKSCDTPPPCPRCPEPKSCDTPPPCPRCPAPELLGGPSVFLFPPKPKDTLMISRTPEVTCVVVDVSHEDPEVQFKWYVDGVEVHNAKTKPREEQYNSTFRVVSVLTVDHHDWLNGKEYKCKVSNKALPAPIEKTISKTKGQPREPQVYTLPPSREEMTKNQVSLTCLVKGFYPSDIAVEWESSGQPENNYNTTPPMLDSDGSFFLYSKLTVDKSRWQQGNIFSCSVMHEALHSHFTQKSLSLSPGK

SEQ ID NO: 16 - protein, 229 aa, synthetic construct
ESKYGPPCPSCPAPEFLGGPSVFLFPPKPKDTLMISRTPEVTCVVVDVSQEDPEVQFNWYVDGVEVHNAKTKPREEQFNSTYRVVSVLTVDHHDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHSHYTQKSLSLSLGK

SEQ ID NO: 17 - protein, 411 aa, synthetic construct
METDTLLLWVLLLWVPGSTGEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVDHHDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHSHYTQKSLSLSPGKGGGGSGGGGSSRGIKGKRQRRISAEGSQACAKGCELCSEVNGCLKCSPKLFILLERNDIRQVGVCLPSCPPGYFDARNPDMNKCIKCKIEHCEACFSHNFCTKCKEGLYLHKGRCYPACPEGSSAAAGTMECSSPGGGGSGGGGSLPETGGHHHHHHHH

SEQ ID NO: 18 - protein, 5 aa, synthetic construct
GGGGS

SEQ ID NO: 19 - protein, 411 aa, synthetic construct
METDTLLLWVLLLWVPGSTGEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVDHHDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHSHYTQKSLSLSPGKGGGGSGGGGSSRGIKGKRQRRISAEGSQACAKGCELCSEVNGCLKCSPKLFILLERNDIRQVGVCLPSCPPGYFDARNPDMNKCIKCKIEHCEACFSHNFCTKCKEGLYLHKGRCYPACPEGSSAANGTMECSSPGGGGSGGGGSLPETGGHHHHHHHH

SEQ ID NO: 20 - protein, 502 aa, synthetic construct
EPKSCDKTHTCPPCPAPELLGGPSVFLFPPKPKDTLMISRTPEVTCVVVDVSHEDPEVKFNWYVDGVEVHNAKTKPREEQYNSTYRVVSVLTVDHHDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHSHYTQKSLSLSPGKGGGGSGGGGSSRGIKGKRQRRISAEGSQACAKGCELCSEVNGCLKCSPKLFILLERNDIRQVGVCLPSCPPGYFDARNPDMNKCIKCKIEHCEACFSHNFCTKCKEGLYLHKGRCYPACPEGSSAANGTMECSSPGGGGSGGGGSSRGIKGKRQRRISAEGSQACAKGCELCSEVNGCLKCSPKLFILLERNDIRQVGVCLPSCPPGYFDARNPDMNKCIKCKIEHCEACFSHNFCTKCKEGLYLHKGRCYPACPEGSSAANGTMECSSP

SEQ ID NO: 21 - protein, 14 aa, synthetic construct
YVDEVDIIGGHEAK

SEQ ID NO: 22 - protein, 12 aa, synthetic construct
RVRRIIGGHEAK

SEQ ID NO: 23 - protein, 12 aa, synthetic construct
RVRRIIGGHEAK

SEQ ID NO: 24 - protein, 10 aa, synthetic construct
GRIIGGHEAK

SEQ ID NO: 25 - protein, 12 aa, synthetic construct
YEVDIIGGHEAK

SEQ ID NO: 26 - protein, 12 aa, synthetic construct
WEHDIIGGHEAK

SEQ ID NO: 27 - protein, 12 aa, synthetic construct
DVADIIGGHEAK

SEQ ID NO: 28 - protein, 12 aa, synthetic construct
DEHDIIGGHEAK

SEQ ID NO: 29 - protein, 12 aa, synthetic construct
DEVDIIGGHEAK

SEQ ID NO: 30 - protein, 12 aa, synthetic construct
DMQDIIGGHEAK

SEQ ID NO: 31 - protein, 12 aa, synthetic construct
LEVDIIGGHEAK

SEQ ID NO: 32 - protein, 12 aa, synthetic construct
LEHDIIGGHEAK

SEQ ID NO: 33 - protein, 12 aa, synthetic construct
VEIDIIGGHEAK

SEQ ID NO: 34 - protein, 12 aa, synthetic construct
VEHDIIGGHEAK

SEQ ID NO: 35 - protein, 12 aa, synthetic construct
IETDIIGGHEAK

SEQ ID NO: 36 - protein, 12 aa, synthetic construct
LETDIIGGHEAK

SEQ ID NO: 37 - protein, 12 aa, synthetic construct
IEADIIGGHEAK

SEQ ID NO: 38 - protein, 232 aa, synthetic construct
EPKSCDKTHTCPPCPAPELLGGPSVFLFPPKPKDTLMISRTPEVTCVVEDVSHEDPEVKFNWYVDGVEVHNAKTKPREEQYNSTYRVVSVLTVDHHDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHSHYTQKSLSLSPGK

SEQ ID NO: 39 - protein, 232 aa, synthetic construct
EPKSCDKTHTCPPCPAPELLGGPSVFLFPPKPKDTLMISRTPEVTCVVEDVSHEDPEVKFNWYVDGVEVHNAKTKPREEQYNSTYRVVSVLTVDHHDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHYHYTQKSLSLSPGK

SEQ ID NO: 40 - protein, 232 aa, synthetic construct
EPKSCDKTHTCPPCPAPELLGGPSVFLFPPKPKDTLMISRTPEVTCVVVDVSHEDPEVKFNWYVDGVEVHNAKTKPREEQYNSTYRVVSVLTVDHHDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHYHYTQKSLSLSPGK

SEQ ID NO: 41 - skipped

SEQ ID NO: 42 - protein, 4 aa, synthetic construct
LPET

SEQ ID NO: 43-48 - skipped (6 entries)

SEQ ID NO: 49 - protein, 5 aa, synthetic construct
LPKTG

SEQ ID NO: 50 - protein, 5 aa, synthetic construct
LPATG

SEQ ID NO: 51 - protein, 5 aa, synthetic construct
LPNTG

SEQ ID NO: 52 - protein, 5 aa, synthetic construct
LPETG

SEQ ID NO: 53 - protein, 5 aa, synthetic construct
LPXAG

SEQ ID NO: 54 - protein, 5 aa, synthetic construct
LPNAG

SEQ ID NO: 55 - protein, 5 aa, synthetic construct
LPXTA

SEQ ID NO: 56 - protein, 5 aa, synthetic construct
LPNTA

SEQ ID NO: 57 - protein, 5 aa, synthetic construct
LGXTG

SEQ ID NO: 58 - protein, 5 aa, synthetic construct
LGATG

SEQ ID NO: 59 - protein, 5 aa, synthetic construct
IPXTG

SEQ ID NO: 60 - protein, 5 aa, synthetic construct
IPNTG

SEQ ID NO: 61 - protein, 5 aa, synthetic construct
IPETG

SEQ ID NO: 62 - protein, 6 aa, synthetic construct
LPKTGG

SEQ ID NO: 63 - protein, 6 aa, synthetic construct
LPATGG

SEQ ID NO: 64 - protein, 6 aa, synthetic construct
LPNTGG

SEQ ID NO: 65 - protein, 6 aa, synthetic construct
LPETGG

SEQ ID NO: 66 - protein, 6 aa, synthetic construct
LPXAGG

SEQ ID NO: 67 - protein, 6 aa, synthetic construct
LPNAGG

SEQ ID NO: 68 - protein, 6 aa, synthetic construct
LPXTAG

SEQ ID NO: 69 - protein, 6 aa, synthetic construct
LPNTAG

SEQ ID NO: 70 - protein, 6 aa, synthetic construct
LGXTGG

SEQ ID NO: 71 - protein, 6 aa, synthetic construct
LGATGG

SEQ ID NO: 72 - protein, 6 aa, synthetic construct
IPXTGG

SEQ ID NO: 73 - protein, 6 aa, synthetic construct
IPNTGG

SEQ ID NO: 74 - protein, 6 aa, synthetic construct
IPETGG

SEQ ID NO: 75 - protein, 4 aa, synthetic construct
GGGS

SEQ ID NO: 76 - protein, 384 aa, synthetic construct
EPKSCDKTHTCPPCPAPELLGGPSVFLFPPKPKDTLMISRTPEVTCVVVDVSHEDPEVKFNWYVDGVEVHNAKTKPREEQYNSTYRVVSVLTVDHHDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHSHYTQKSLSLSPGKGGGGSGGGGSSRGIKGKRQRRISAEGSQACAKGCELCSEVNGCLKCSPKLFILLERNDIRQVGVCLPSCPPGYFDARNPDMNKCIKCKIEHCEACFSHNFCTKCKEGLYLHKGRCYPACPEGSSAANGTMECSSPGGGGSGGGGSLPETGGG

SEQ ID NO: 77 - protein, 385 aa, synthetic construct
EPKSCDKTHTCPPCPAPELLGGPSVFLFPPKPKDTLMISRTPEVTCVVVDVSHEDPEVKFNWYVDGVEVHNAKTKPREEQYNSTYRVVSVLTVDHHDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHSHYTQKSLSLSPGKGGGGSGGGGSSRGIKGKRQRRISAEGSQACAKGCELCSEVNGCLKCSPKLFILLERNDIRQVGVCLPSCPPGYFDARNPDMNKCIKCKIEHCEACFSHNFCTKCKEGLYLHKGRCYPACPEGSSAANGTMECSSPGGGGSGGGGSLPETGGGV

SEQ ID NO: 78 - protein, 52 aa, synthetic construct
SYVSNPICKGCLSCSKDNGCSRCQQKLFFFLRREGMRQYGECLHSCPSGYYG

SEQ ID NO: 79 - protein, 45 aa, synthetic construct
MNRCARCRIENCDSCFSKDFCTKCKVGFYLHRGRCFDECPDGFAP

SEQ ID NO: 80 - protein, 52 aa, synthetic construct
PNVSQGCQGGCATCSDYNGCLSCKPRLFFALERIGMKQIGVCLSSCPSGYYG

SEQ ID NO: 81 - protein, 44 aa, synthetic construct
INKCTKCKADCDTCFNKNFCTKCKSGFYLHLGKCLDNCPEGLEA

SEQ ID NO: 82 - protein, 54 aa, synthetic construct
GTGLGGNCTGCIICSEENGCSTCQQRLFLFIRREGIRQYGKCLHDCPPGYFGIR

SEQ ID NO: 83 - protein, 44 aa, synthetic construct
VNRCKKCGATCESCFSQDFCIRCKRQFYLYKGKCLPTCPPGTLA

SEQ ID NO: 84 - protein, 15 aa, synthetic construct
EAAAKEAAAKEAAAK

SEQ ID NO: 85 - protein, 102 aa, synthetic construct
SYVSNPICKGCLSCSKDNGCSRCQQKLFFFLRREGMRQYGECLHSCPSGYYGHRAPDMNRCARCRIENCDSCFSKDFCTKCKVGFYLHRGRCFDECPDGFAP

SEQ ID NO: 86 - protein, 101 aa, synthetic construct
PNVSQGCQGGCATCSDYNGCLSCKPRLFFALERIGMKQIGVCLSSCPSGYYGTRYPDINKCTKCKADCDTCFNKNFCTKCKSGFYLHLGKCLDNCPEGLEA

SEQ ID NO: 87 - protein, 101 aa, synthetic construct
GTGLGGNCTGCIICSEENGCSTCQQRLFLFIRREGIRQYGKCLHDCPPGYFGIRGQEVNRCKKCGATCESCFSQDFCIRCKRQFYLYKGKCLPTCPPGTLA

SEQ ID NO: 88 - protein, 52 aa, synthetic construct
AEGSQACAKGCELCSEVNGCLKCSPKLFILLERNDIRQVGVCLPSCPPGYFD

SEQ ID NO: 89 - protein, 45 aa, synthetic construct
MNKCIKCKIEHCEACFSHNFCTKCKEGLYLHKGRCYPACPEGSSA

SEQ ID NO: 90 - protein, 125 aa, synthetic construct
SRGIKGKAQRRISAEGSQACAKGCELCSEVNGCLKCSPKLFILLERNDIRQVGVCLPSCPPGYFDARNPDMNKCIKCKIEHCEACFSHNFCTKCKEGLYLHKGRCYPACPEGSSAANGTMECSSP

SEQ ID NO: 91 - protein, 125 aa, synthetic construct
SRGIKGKRQARISAEGSQACAKGCELCSEVNGCLKCSPKLFILLERNDIRQVGVCLPSCPPGYFDARNPDMNKCIKCKIEHCEACFSHNFCTKCKEGLYLHKGRCYPACPEGSSAANGTMECSSP

SEQ ID NO: 92 - protein, 115 aa, synthetic construct
SISAEGSQACAKGCELCSEVNGCLKCSPKLFILLERNDIRQVGVCLPSCPPGYFDARNPDMNKCIKCKIEHCEACFSHNFCTKCKEGLYLHKGRCYPACPEGSSAANGTMECSSP

SEQ ID NO: 93 - protein, 118 aa, synthetic construct
SRGIISAEGSQACAKGCELCSEVNGCLKCSPKLFILLERNDIRQVGVCLPSCPPGYFDARNPDMNKCIKCKIEHCEACFSHNFCTKCKEGLYLHKGRCYPACPEGSSAANGTMECSSP

SEQ ID NO: 94 - protein, 121 aa, synthetic construct
SRGIKGKISAEGSQACAKGCELCSEVNGCLKCSPKLFILLERNDIRQVGVCLPSCPPGYFDARNPDMNKCIKCKIEHCEACFSHNFCTKCKEGLYLHKGRCYPACPEGSSAANGTMECSSP

SEQ ID NO: 95 - protein, 119 aa, synthetic construct
SRQRRISAEGSQACAKGCELCSEVNGCLKCSPKLFILLERNDIRQVGVCLPSCPPGYFDARNPDMNKCIKCKIEHCEACFSHNFCTKCKEGLYLHKGRCYPACPEGSSAANGTMECSSP

SEQ ID NO: 96 - protein, 107 aa, synthetic construct
ACAKGCELCSEVNGCLKCSPKLFILLERNDIRQVGVCLPSCPPGYFDARNPDMNKCIKCKIEHCEACFSHNFCTKCKEGLYLHKGRCYPACPEGSSAANGTMECSSP

SEQ ID NO: 97 - DNA, 1257 bp, synthetic construct
  source 1..1257: mol_type = other DNA
gctagcgccaccatggaaaccgatacactgctgctgtgggtgctgctcctttgggtgcccggatctacaggcgagcctaagagctgcgacaagacccacacctgtcctccatgtcctgctccagaactgctcggcggaccttccgtgttcctgtttcctccaaagcctaaggacaccctgatgatcagcagaacccctgaagtgacctgcgtggtggtggatgtgtcccacgaggatcccgaagtgaagttcaattggtacgtggacggcgtggaagtgcacaacgccaagaccaagcctagagaggaacagtacaacagcacctacagagtggtgtccgtgctgaccgtggaccaccacgattggctgaacggcaaagagtacaagtgcaaggtgtccaacaaggccctgcctgctcctatcgagaaaaccatcagcaaggccaagggccagcctagggaaccccaggtttacacactgcctccaagcagggacgagctgaccaagaatcaggtgtccctgacctgcctggtcaagggcttctacccttccgatatcgccgtggaatgggagagcaatggccagcctgagaacaactacaagacaacccctcctgtgctggacagcgacggctcattcttcctgtacagcaagctgacagtggacaagagcagatggcagcagggcaacgtgttcagctgcagcgtgatgcacgaagccctgcacagccactacacccagaagtccctgtctctgagccctggaaaaggcggcggaggatctggcggaggcggatcttctagaggcatcaagggcaaggcccagcggagaatctctgccgagggatctcaggcctgtgccaagggctgtgaactgtgcagcgaagtgaacggctgcctgaagtgcagccccaagctgttcatcctgctggaacggaacgacatccggcaagtgggagtgtgcctgccttcttgtcctcctggctacttcgacgccagaaatcccgacatgaacaagtgcattaagtgcaagatcgagcactgcgaggcctgcttcagccacaacttctgcaccaagtgcaaagagggcctgtacctgcacaagggcagatgctatcctgcctgtcctgagggaagctctgccgccaatggcacaatggaatgtagctctccaggtggcggaggaagcggtggcggcggatctcttccagaaacaggcggacaccatcaccatcatcaccaccactgatgactcgag

SEQ ID NO: 98 - protein, 415 aa, synthetic construct
ASATMETDTLLLWVLLLWVPGSTGEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVDHHDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHSHYTQKSLSLSPGKGGGGSGGGGSSRGIKGKAQRRISAEGSQACAKGCELCSEVNGCLKCSPKLFILLERNDIRQVGVCLPSCPPGYFDARNPDMNKCIKCKIEHCEACFSHNFCTKCKEGLYLHKGRCYPACPEGSSAANGTMECSSPGGGGSGGGGSLPETGGHHHHHHHH

SEQ ID NO: 99 - DNA, 1257 bp, synthetic construct
  source 1..1257: mol_type = other DNA
gctagcgccaccatggaaaccgatacactgctgctgtgggtgctgctcctttgggtgcccggatctacaggcgagcctaagagctgcgacaagacccacacctgtcctccatgtcctgctccagaactgctcggcggaccttccgtgttcctgtttcctccaaagcctaaggacaccctgatgatcagcagaacccctgaagtgacctgcgtggtggtggatgtgtcccacgaggatcccgaagtgaagttcaattggtacgtggacggcgtggaagtgcacaacgccaagaccaagcctagagaggaacagtacaacagcacctacagagtggtgtccgtgctgaccgtggaccaccacgattggctgaacggcaaagagtacaagtgcaaggtgtccaacaaggccctgcctgctcctatcgagaaaaccatcagcaaggccaagggccagcctagggaaccccaggtttacacactgcctccaagcagggacgagctgaccaagaatcaggtgtccctgacctgcctggtcaagggcttctacccttccgatatcgccgtggaatgggagagcaatggccagcctgagaacaactacaagacaacccctcctgtgctggacagcgacggctcattcttcctgtacagcaagctgacagtggacaagagcagatggcagcagggcaacgtgttcagctgcagcgtgatgcacgaagccctgcacagccactacacccagaagtccctgtctctgagccctggaaaaggcggcggaggatctggcggaggcggatcttctagaggcatcaagggcaagagacaggccagaatctctgccgagggatctcaggcctgtgccaagggctgtgaactgtgcagcgaagtgaacggctgcctgaagtgcagccccaagctgttcatcctgctggaacggaacgacatccggcaagtgggagtgtgcctgccttcttgtcctcctggctacttcgacgccagaaatcccgacatgaacaagtgcattaagtgcaagatcgagcactgcgaggcctgcttcagccacaacttctgcaccaagtgcaaagagggcctgtacctgcacaagggcagatgctatcctgcctgtcctgagggaagctctgccgccaatggcacaatggaatgtagctctccaggtggcggaggaagcggtggcggcggatctcttccagaaacaggcggacaccatcaccatcatcaccaccactgatgactcgag

SEQ ID NO: 100 - DNA, 375 bp, synthetic construct
  source 1..375: mol_type = other DNA
tctagaggcatcaagggcaagagacaggccagaatctctgccgagggatctcaggcctgtgccaagggctgtgaactgtgcagcgaagtgaacggctgcctgaagtgcagccccaagctgttcatcctgctggaacggaacgacatccggcaagtgggagtgtgcctgccttcttgtcctcctggctacttcgacgccagaaatcccgacatgaacaagtgcattaagtgcaagatcgagcactgcgaggcctgcttcagccacaacttctgcaccaagtgcaaagagggcctgtacctgcacaagggcagatgctatcctgcctgtcctgagggaagctctgccgccaatggcacaatggaatgtagctctcca

SEQ ID NO: 101 - DNA, 1257 bp, synthetic construct; identical to SEQ ID NO: 99
  source 1..1257: mol_type = other DNA

SEQ ID NO: 102 - protein, 419 aa, synthetic construct
  UNSURE 416..417
ASATMETDTLLLWVLLLWVPGSTGEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVDHHDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHSHYTQKSLSLSPGKGGGGSGGGGSSRGIKGKRQARISAEGSQACAKGCELCSEVNGCLKCSPKLFILLERNDIRQVGVCLPSCPPGYFDARNPDMNKCIKCKIEHCEACFSHNFCTKCKEGLYLHKGRCYPACPEGSSAANGTMECSSPGGGGSGGGGSLPETGGHHHHHHHHXXLE

SEQ ID NO: 103 - DNA, 1269 bp, synthetic construct
  source 1..1269: mol_type = other DNA
agcagcgctagcgccaccatggaaaccgatacactgctgctgtgggtgctgctcctttgggtgcccggatctacaggcgagcctaagagctgcgacaagacccacacctgtcctccatgtcctgctccagaactgctcggcggaccttccgtgttcctgtttcctccaaagcctaaggacaccctgatgatcagcagaacccctgaagtgacctgcgtggtggtggatgtgtcccacgaggatcccgaagtgaagttcaattggtacgtggacggcgtggaagtgcacaacgccaagaccaagcctagagaggaacagtacaacagcacctacagagtggtgtccgtgctgaccgtggaccaccacgattggctgaacggcaaagagtacaagtgcaaggtgtccaacaaggccctgcctgctcctatcgagaaaaccatcagcaaggccaagggccagcctagggaaccccaggtttacacactgcctccaagcagggacgagctgaccaagaatcaggtgtccctgacctgcctggtcaagggcttctacccttccgatatcgccgtggaatgggagagcaatggccagcctgagaacaactacaagacaacccctcctgtgctggacagcgacggctcattcttcctgtacagcaagctgacagtggacaagagcagatggcagcagggcaacgtgttcagctgcagcgtgatgcacgaagccctgcacagccactacacccagaagtccctgtctctgagccctggaaaaggcggcggaggatctggcggaggcggatcttctagaggcatcaagggcaagagacagcggagaatctctgccgagggatctcaggcctgtgccaagggctgtgaactgtgcagcgaagtgaacggctgcctgaagtgcagccccaagctgttcatcctgctggaacggaacgacatccggcaagtgggagtgtgcctgccttcttgtcctcctggctacttcgacgccagaaatcccgacatgaacaagtgcattaagtgcaagatcgagcactgcgaggcctgcttcagccacaacttctgcaccaagtgcaaagagggcctgtacctgcacaagggcagatgctatcctgcctgtcctgagggaagctctgccgccaatggcacaatggaatgtagctctccaggtggcggaggaagcggtggcggcggatctcttccagaaacaggcggacaccatcaccatcatcaccaccactgatgactcgagagcagc

SEQ ID NO: 104 - skipped

SEQ ID NO: 105 - protein, 546 aa, synthetic construct
METDTLLLWVLLLWVPGSTGEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVDHHDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHSHYTQKSLSLSPGKGGGGSGGGGSSRGIKGKRQRRISAEGSQACAKGCELCSEVNGCLKCSPKLFILLERNDIRQVGVCLPSCPPGYFDARNPDMNKCIKCKIEHCEACFSHNFCTKCKEGLYLHKGRCYPACPEGSSAANGTMECSSPGGGGSGGGGSSRGIKGKRQRRISAEGSQACAKGCELCSEVNGCLKCSPKLFILLERNDIRQVGVCLPSCPPGYFDARNPDMNKCIKCKIEHCEACFSHNFCTKCKEGLYLHKGRCYPACPEGSSAANGTMECSSPGGGGSGGGGSLPETGGHHHHHHHH

SEQ ID NO: 106 - protein, 6 aa, synthetic construct
LAHTGG